(19) United States
(12) Patent Application Publication
(10) Pub. No.: US 2023/0285278 A1
(43) Pub. Date: Sep. 14, 2023

(54) METHODS FOR TREATING CHRONIC RHINOSINUSITIS

(71) Applicant: OptiNose, Inc., Yardley, PA (US)

(72) Inventors: Ramy MAHMOUD, Yardley, PA (US); John MESSINA, Yardley, PA (US)

(21) Appl. No.: 18/179,178

(22) Filed: Mar. 6, 2023

Related U.S. Application Data
(60) Provisional application No. 63/317,067, filed on Mar. 6, 2022.
(60) Provisional application No. 63/351,421, filed on Jun. 12, 2022.
(60) Provisional application No. 63/355,961, filed on Jun. 27, 2022.
(60) Provisional application No. 63/388,594, filed on Jul. 12, 2022.

(51) Int. Cl. A61K 9/00; A61K 31/569; A61P 11/02; A61M 15/08; A61M 15/00

(52) U.S. Cl. CPC A61K 9/0043; A61K 31/569; A61P 11/02; A61M 15/085; A61M 15/0021

(57) ABSTRACT

A method of delivering a substance, such as fluticasone propionate, a nasal steroid and/or carbon dioxide gas, to the nasal cavity of a subject, in particular for the treatment of sinus and/or nasal inflammatory diseases, for example, chronic rhinosinusitis with or without nasal polyps, to reduce sinus inflammation, reduce sinus opacification, and/or improve associated symptoms.

### CROSS-REFERENCE TO RELATED APPLICATIONS

This application claims priority to U.S. Provisional Application No. 63/317,067, filed Mar. 6, 2022, U.S. Provisional Application No. 63/351,421, filed Jun. 12, 2022, U.S. Provisional Application No. 63/355,961, filed Jun. 27, 2022, and U.S. Provisional Application No. 63/388,594, filed Jul. 12, 2022. The contents of these applications are each incorporated herein by reference in their entirety.

### INTRODUCTION

### Field

The present disclosure relates to the nasal administration of substances, in particular drugs, and in particular substances which may benefit from a rapid onset of action or which may benefit from topical activity, in particular topical activity in the upper posterior of a subject's nasal cavity, such as in the treatment of—or the treatment of the symptoms of—sinus and/or nasal inflammatory diseases, for example, chronic rhinosinusitis with or without nasal polyps. The present disclosure also relates to nasal delivery of carbon dioxide gas, adjustment of nasal cavity pH adjustment, or adjustment of nasal cavity NO concentration, solely or as a supplement to nasal administration of substances as a therapeutic treatment, such as for the treatment of—or the treatment of the symptoms of—sinus and/or nasal inflammatory diseases, for example, chronic rhinosinusitis with or without nasal polyps. This effect associated with carbon dioxide when using the breath powered nasal delivery devices may, in combination with one or more other factors, include positive air pressure, high flow rate and changed flow pattern of gases through a subject's nasal cavity and/or sinuses, improved gas flow, e.g., exhalation breath, penetrating a nasal airway, vibratory effect in operating the devices, and removal of nitric oxide in a subject's nasal cavity and/or sinuses can cause stimulatory or mediating effects on the trigeminal nerve and on mast cells. The present disclosure further relates to nasal delivery of substances or nasal delivery of carbon dioxide gas or nasal pH adjustment, solely or as a supplement to nasal administration of substances, as a therapeutic treatment, such as for the treatment of nasal or sinus inflammation.

### Introduction

Sinusitis is a symptomatic inflammation of the paranasal sinuses and nasal cavity. If the duration of sinusitis is more than 12 weeks, with or without acute exacerbations, it is deemed to be “chronic rhinosinusitis” (CRS) (Fokkens et al., 2020; Orlandi et al., 2021). Although not recognized in International Classification of Diseases Version 10 (ICD-10), the term “chronic rhinosinusitis” is used to recognize that patients with chronic sinusitis (inflammation in the sinus cavities) also typically have inflammation in the nasal passages (“rhinitis”) and associated nasal symptoms (e.g., congestion, nasal discharge). Chronic sinusitis (CS) and CRS are generally treated as synonyms and refer to patients with chronic symptomatic sinus and/or nasal inflammation.

Persistent inflammation of the paranasal sinuses and/or the nasal cavity, progressing from mucosal injury to tissue change, is sine qua non in the pathophysiology of CS (Akdis et al., 2013). Evidence of persistent inflammation is generally present in the sinuses and throughout the nasal cavity, particularly including the region of the ostiomeatal complex (OMC), where the sinuses ventilate and drain into the nasal cavity.

Generally, the four defining symptoms of chronic rhinosinusitis, irrespective of the presence or absence of nasal polyps (NP), are nasal obstruction/congestion/blockage, nasal drainage (mucopurulent) that may drain anteriorly or posteriorly, facial pain/pressure/fullness, and decreased or loss of sense of smell (Rosenfeld et al., 2015; Fokkens et al., 2020; Orlandi et al., 2021).

Patients with CRS often experience periodic acute exacerbations thought to most often be of infectious origin and to be a consequence of impaired mucociliary clearance due to chronic inflammation of the mucosal surface (Orlandi et al., 20211. Acute exacerbations of CRS (AECRS) are a major source of disability for the roughly 30 million patients in the United States who suffer from CS, and not only contribute directly to perceived lack of disease control and poor quality of life but also often push people to frequent use of antibiotics, oral steroids, or even surgery. In the context of the approximately 10 million office visits coded for CRS every year, approximately 70% result in an antibiotic being prescribed, making CRS potentially the single most common reason for adult outpatient antibiotic use (Smith et al., 2013). Frequent use of antibiotics not only poses risks to the individuals using them, including adverse drug reactions and adverse changes to the microbiome of the nose or gut, but also societal risks, such as emergence and dissemination of drug-resistant organisms.

A wide range of inflammatory stimuli may act together with mucociliary and/or structural abnormalities to give rise to the development of the syndrome of CRS. The multifactorial etiology of CRS, involving genetic factors, environmental influences, occupational factors, infection (viral and/or bacterial), allergy, immune dysfunction, and systemic diseases, and comparatively recent elucidation of inflammatory cytokine profiles, has led to discussion of inflammatory endotypes of disease. CRS has been found to be accompanied by various inflammatory clusters, including type 1 (T1) driven (sometimes labeled as neutrophilic) inflammation, type 2 (T2) driven (sometimes labeled as eosinophilic) inflammation, neurogenic, epithelial, and mixed endotypes (Borish and Hamilos, 2019).

Stevens et al. (2019) addressed the question of whether inflammatory endotypes found within CRS tissue correspond to distinct clinical presentations (i.e., the historically used visual phenotypes of CRSwNP and chronic rhinosinusitis without nasal polyps [CRSsNP], which are based on endoscopic assessment of the nasal cavity for the presence of polyps). The investigators examined sinus tissue from patients with CRS and defined T1, T2, and type 3 (T3) disease. Consistent with previous studies, the presence of NP usually correlated with a T2 profile at the population level. Stevens reported that 87% of polypoid CRS had evidence of T2 inflammation, though only 62% had exclusively T2 inflammation. However, what is crucial when considering whether endotype predicts phenotype, particularly in an individual patient, is that T2 inflammation was also the most common endotype in non-polyp CRS. Stevens and colleagues demonstrated that a majority (55%) of nonpolyp CRS cases had evidence of T2 inflammation, and 34% had evidence of only T2 inflammation. Thus, irrespective of whether or not polyps are present in the nasal cavity, more than half of patients with CRS have T2 inflammation or a mix of inflammation including T2 inflammation.

It is recognized by multiple authors and studies that the simple phenotypic distinction of CRS as being “with or without NP” is a poor predictor of inflammatory endotype, especially at the individual level, because of the highly overlapping molecular pathology of the 2 disease phenotypes (Kato, 2015; Pezato et al., 2019; Brown et al., 2021; Kato et al., 2022). It seems increasingly apparent that factors other than endotype, such as tissue structure and hydrostatic forces, likely contribute to the emergence, or not, of polyps in the nasal cavity of any individual patient. This may explain known features of the phenotypes, such as the lack of polyp formation on respiratory epithelium of the inferior turbinate and significant population differences by geography, that are inconsistent with the hypothesis that endotype corresponds with phenotype. In a single CRS patient, regardless of the presence or absence of polyps in the nasal cavity, pin-pointing the different etiologic factors responsible for the development of the disease remains a challenge.

In summary, there appears to be highly overlapping biology and pathophysiology of patients with chronic sinusitis with or without polyps in the nasal cavity. Both phenotypes have now been shown to exhibit heterogeneous inflammatory endotypes, with T2 inflammatory markers in more than half of CS patients with or without polyps in the nasal cavity. Both phenotypes are also generally characterized by inflammation throughout the deep sino-nasal regions (“rhino-sinusitis”), including the sinuses and the OMC of the nasal cavity. The similarity of these phenotypes is reinforced by the fact that the condition “nasal polyps” is diagnosed based on visual inspection of the nasal cavity, but polyps are sometimes observed in the sinuses of patients without polyps in the nasal cavity when sinus surgery is performed to open the paranasal sinuses and enable inspection of the sinus spaces. Moreover, the primary diagnostic presenting symptoms for these 2 conditions are identical. Thus, these phenotypes are closely related both biologically and in practice. The primary distinction, particularly in clinical practice, is anatomic: “nasal polyps” (or CRSwNP) refers to the endoscopically observable condition of having polyposis inside the nasal cavity while “chronic sinusitis” (without visible polyps in the nasal cavity) refers to inflammatory disease inside the sinus cavities, typically diagnosed by imaging such as computed tomography (CT) scan. The 2 phenotypes (i.e., nasal disease marked by polyposis in the nasal cavity and sinus disease marked by inflammation in the sinus cavities) may present in the same patient at different times throughout the course of their disease process.

Chronic rhinosinusitis is a potentially disabling disease that creates a high degree of population morbidity, including a substantial direct and indirect burden on society, due to an exceptionally high disease prevalence and a large negative effect on the day-to-day functioning and quality of life of people with the disease. Chronic sinusitis is reported to be the second most prevalent chronic health condition of adults in the United States, affecting ˜12.5% of the population or over 30 million patients each year (Adams et al., 1999; Anand, 2004; Hamilos, 2011). It also drives an enormous burden of healthcare utilization, including approximately 10 million outpatient visits for CRS every year, of which approximately 70% result in prescription of an antibiotic (Smith et al., 2013). In addition, AECRS can also trigger asthma exacerbations which can be associated with serious clinical consequences.

Gas therapy has been described as a remedy for CRS-associated conditions and co-morbidities, including headaches, allergies, and asthma, as well as associated physiologies (Casale et al., J Allergy Clin Immunol 121 (1): 105-109 (2008), Vause et al., Headache 47: 1385-1397 (2007), Tzabazis et al., Life Science 87: 36-41 (2010), and Casale et al., Ann Allergy Asthma Immunol 107: 364-370 (2011)). Further, WO-A-2001/064280 discloses methods and devices for transcutaneous and transmucosal applications of carbon dioxide in the form of a gas and in the form of a capnic solution (such as carbonated water) for the relief of pain, including musculoskeletal disorders, neuralgias, rhinitis and other ailments. Additionally, US-A-2011/0046546 discloses apparatuses, methods and kits for treating symptoms associated with common ailments, such as headaches, rhinitis, asthma, epilepsy, nervous disorders and the like.

However, there are no Food and Drug Administration (FDA)-approved medications for the treatment of CRS generally and, notably, the more common presentation of CRS without concurrent polyps in the nasal cavity, even though it has such a high prevalence; current FDA-approved medications are approved only for the treatment of nasal polyps. As a consequence, physicians frequently prescribe off-label medications with limited evidence of safety or efficacy in an effort to relieve patient suffering. Absent alternatives, many patients (estimated over 600,000 per year) undergo surgery that is associated with a variety of risks, or commit to frequent use of systemic corticosteroids despite known risks, which, particularly for patients with NP, are increasingly escalated to parenteral monoclonal antibody treatment. While surgery can result in symptom relief, a large proportion of patients continue to require medical therapy after surgery for either incomplete symptom control or recurrent symptoms.

Poor sleep quality and severe fatigue are also associated with CRS that have substantial impact on patient quality of life. A trial conducted using the Pittsburgh Sleep Quality Index (PSQI) (Alt et al., 2013) in patients with CRS found that 75% of patients reported “poor” sleep based on accepted cutoffs, while a separate study found that the median prevalence of fatigue was 54% (11% to 73%) (Chester et al., 2008). The secondary consequences of poor sleep quality and fatigue can be far reaching and include metabolic disorders such as diabetes and obesity (Knutson et al., 2007), and, among older individuals, an increased incidence of balance, ambulatory, and vision difficulties along with higher rates of falls and increased overall mortality (Neikrug and Ancoli-Israel, 2010).

No medication has been approved based on demonstration of improvement of CRS symptoms in the full spectrum of patients with CRS (i.e., CRS with and without nasal polyps), and no medication has presented primary endpoints demonstrating evidence of efficacy for both symptoms and reduction of inflammation inside the sinus cavities, i.e., evidence of a biologic effect in the anatomic location inside the paranasal sinuses, especially of patients with CRSsNP. In particular, no drug has shown substantial evidence for either reduction of symptoms or reduction of intra-sinus inflammation for patients suffering from CRSsNP.

Surprisingly, small placebo-controlled trials evaluating symptom benefits of standard-delivery nasal steroid sprays for CRSsNP found no significant benefit with standard-delivery fluticasone nasal spray (Parikh et al., 2001) and no significant benefit with standard-delivery mometasone nasal spray (Mosges et al., 2011; Dixon et al., 2015). Although 1 of 2 trials with standard budesonide nasal spray found symptom benefit, the benefit derived was only from the subgroup of allergic patients (Lund et al., 2004; Qvarnberg et al., 1992). Systematic reviews and meta-analyses suggest small symptom benefits with standard-delivery nasal steroid spray treatment of CRS, primarily driven by CRSwNP patients, and “little effect” on quality-of-life (Fokkens et al., 2020; Orlandi et al., 2021). Evidence for reducing AECRS with standard-delivery steroid nasal sprays is very limited, with data from 2 trials suggesting no benefit (Dijkstra et al., 2004; Lund et al., 2004).

By far, the most commonly used method of delivery for intranasal steroids is traditional nasal sprays, though “standard-delivery” nasal spray has repeatedly been shown to be suboptimal for delivery of topically acting drugs to sites in the superior and posterior regions of the nasal passages, notably to sinus drainage tract sites both behind the nasal valve and above the inferior turbinate and in the middle meatus/OMC (Leach et al., 2015; Sui et al., 2019). The OMC is located deep in the nasal cavity and is the region where the paranasal sinuses normally drain and ventilate (Aggarwal et al., 2004; Larsen and Tos, 2004; Djupesland, 2013). Topical steroid treatment of this superior/posterior portion of the nasal labyrinth, potentially including sinus ostia or into sinus cavities depending on surgical status, and off-label use of steroid rinses containing a high dose of liquid steroid (for example, with saline and added budesonide with “Pulmicort Respules”) have been minimally studied (Bernstein et al., 2023).

The limitations of current treatment options leave significant unmet medical need for an approved medication that enables evidence-based treatment of this common chronic disease without escalation to surgery, ideally a medication with a risk/benefit profile supporting use in a broad cross-section of patients, and that can improve the symptoms and risk of acute exacerbations associated with CRS regardless of presence of polyps in the nasal cavity, and treat sinus inflammation or disease, and improve or reduce inflammation in the sinuses. The inventors unexpectedly found that the present disclosure addresses these shortcomings.

### SUMMARY

Referring to FIG. 1, the nasal airway 1 comprises the two nasal cavities separated by the nasal septum, which airway 1 includes numerous ostia, such as the paranasal sinus ostia 3 and the tubal ostia 5, and olfactory cells, and is lined by the nasal mucosa. The nasal airway 1 can communicate with the nasopharynx 7, the oral cavity 9 and the lower airway 11, with the nasal airway 1 being in selective communication with the anterior region of the nasopharynx 7 and the oral cavity 9 by opening and closing of the oropharyngeal velum 13. The velum 13, which is often referred to as the soft palate, is illustrated in solid line in the closed position, as achieved by providing a certain positive pressure in the oral cavity 9, such as achieved by balancing pressures in the oral cavity 9 and the nasal airway 1 on exhalation through the oral cavity 9, and in dashed line in the open position.

The posterior region of the nasal airway is that region which is posterior of the nasal valve NV, as illustrated in FIG. 2. The nasal valve comprises the anterior bony cavum which contains inferior turbinate erectile tissue and septal erectile tissue, which are supported respectively by compliant ala tissue and the rigid cartilaginous septum (Cole, P (The Respiratory Role of the Upper Airways, a selective clinical and pathophysiological review. 1993, Mosby-Year Book Inc. ISBN1.55664-390-X)). These elements combine to form a dynamic valve, which extends over several millimeters, that adjusts nasal airflow, and is stabilized by cartilage and bone, modulated by voluntary muscle and regulated by erectile tissue. The lumen of the nasal valve is the section of narrowest cross-sectional area between the posterior and anterior regions of the nasal airway, and is much longer and narrower dorsally than ventrally, and this lumen defines a triangular entrance which extends to the piriform region of the bony cavum. The nasal valve is lined in its anterior part with transitional epithelium, with a gradual transition posterior to respiratory epithelium. The nasal valve and anterior vestibule define roughly the anterior one-third of the nose.

The posterior region of the nasal airway is that region which is lined with respiratory epithelium, which is ciliated, and olfactory epithelium, which comprises nerves which extend downwards through the cribiform plate CP from the olfactory bulb, whereas the anterior region of the nasal airway is that region which is lined with squamous epithelium, which is not ciliated, and transitional epithelium. The olfactory epithelium extends on both the lateral and medial sides of the nasal airway, and typically extends downwards about 1.5 to 2.5 cm.

The upper posterior region is the region above the inferior meatus (IM), as illustrated in FIG. 2, and encompasses the middle turbinate, the sinus ostia in infundibulum (ostia to maxillary, frontal and ethmoidal sinuses), the olfactory region, and the upper branches of the trigeminal nerve, and is that region which includes veins which drain to the venous sinuses that surround the brain.

As illustrated in FIG. 2, the posterior region of the nasal airway is the nasal region posterior of an imaginary vertical plane VERT1 which is located at a position corresponding to one-quarter of the distance between the anterior nasal spine (AnS), which is a pointed projection at the anterior extremity of the intermaxillary suture, and the posterior nasal spine (PnS), which is the sharp posterior extremity of the nasal crest of the hard palate and represents the transition between the nose and the nasopharynx, which corresponds to a distance posterior of the AnS of between about 13 mm and about 14 mm (Rosenberger, H (Growth and Development of the Naso-Respiratory Area in Childhood, PhD Thesis, Laboratory of Anatomy, School of Medicine, Western Reserve University, Presented to the Annual Meeting of the American Laryngological, Rhinological and Otological Society, Charleston, S.C., USA, 1934) defines the distance between the anterior nasal spine AnS and the PnS as being 56 mm in eighteen year old boys and 53.3 mm in eighteen year old girls). As again illustrated in FIG. 2, the posterior nasal region is bounded posteriorly by an imaginary vertical plane VERT2 which extends through the PnS.

As further illustrated in FIG. 2, the upper region of the nasal airway is an upper segment of the nasal airway which is bounded by the cribiform plate (CP) and a horizontal plane HORIZ which is located at a position corresponding to one-third of the distance between the nasal floor (NF) of the nasal airway and the CP, which corresponds to a height of typically between about 13 and about 19 mm above the NF (Zacharek, M A et al. (Sagittal and Coronal Dimensions of the Ethmoid Roof: A Radioanatomic Study, Am J Rhinol 2005, Vol 19, pages 348 to 352) define the distance from the NF to the CP as 46+/−4 mm). The upper posterior region can thus include an upper posterior region which may be bounded by the above-defined vertical and horizontal planes VERT1, HORIZ.

### BRIEF DESCRIPTION OF DRAWING(S)

FIG. 1 illustrates the anatomy of the upper respiratory tract of a human subject.

FIG. 2 depicts the segmentation of a nasal cavity.

FIGS. 3A-3B illustrate a Breath Powered™ Bi-Directional™ nasal delivery device in accordance with the present disclosure.

FIGS. 4A-4E illustrate alternative detailed views of a Breath Powered™ Bi-Directional™ nasal delivery device in accordance with the present disclosure.

FIG. 5 depicts an overview of the study design for the 3205 (Study A; CRS with and without nasal polyps) and 3206 (Study B; CRS without nasal polyps) clinical studies.

FIGS. 6A-6D illustrate the change from baseline in individual cardinal symptoms of CRS in study 3206 (Study B; CRS without nasal polyps). Data from the placebo group are represented by square symbols, data from the OPN-375 186 μg BID group are represented by triangle symbols, and data from the OPN-375 372 μg BID group are represented by circle symbols.

FIG. 7 depicts the least squares (LS) mean change from baseline in the Sinonasal outcome test-22 score over time for study 3206 (Study B; CRS without nasal polyps). Data from the placebo group are represented by square symbols, data from the OPN-375 186 μg BID group are represented by triangle symbols, and data from the OPN-375 372 μg BID group are represented by circle symbols.

FIG. 8 depicts the change in composite symptom score over time for study 3205 (Study A; CRS with and without nasal polyps). Data from the placebo group are represented by square symbols, data from the OPN-375 186 μg BID group are represented by triangle symbols, and data from the OPN-375 372 μg BID group are represented by circle symbols.

FIGS. 9A-9D depicts the change in individual cardinal symptoms over time for study 3205 (Study A; CRS with and without nasal polyps). Data from the placebo group are represented by square symbols, data from the OPN-375 186 μg BID group are represented by triangle symbols, and data from the OPN-375 372 μg BID group are represented by circle symbols.

FIG. 10 illustrates the change in the Sinonasal outcome test-22 (SNOT-22) total score over time for study 3205 (Study A; CRS with and without nasal polyps). Data from the placebo group are represented by square symbols, data from the OPN-375 186 μg BID group are represented by triangle symbols, and data from the OPN-375 372 μg BID group are represented by circle symbols.

FIG. 11 depicts the subject disposition for subjects participating in study 3205 (Study A; CRS with and without nasal polyps) and 3206 (Study B; CRS without nasal polyps).

FIGS. 12A-12B show the changes from baseline to the end of week 4 in instantaneous morning (AM) composite symptoms score by subgroups.

FIGS. 13A-13B show the changes from baseline to week 24/early termination (ET) in the average of percentages of opacified volume in the ethmoid and maxillary sinuses by subgroup.

FIGS. 14A-14B show a comparison of deposition of an agent using a standard-delivery nasal spray (FIG. 14A) and the exhalation delivery device as herein described incorporating the same standard-delivery nasal spray (FIG. 14B). FIGS. 14A-14B are reproduced from Djupesland, P. G., 2013 under the terms of the Creative Commons Attribution 2.0 International License.

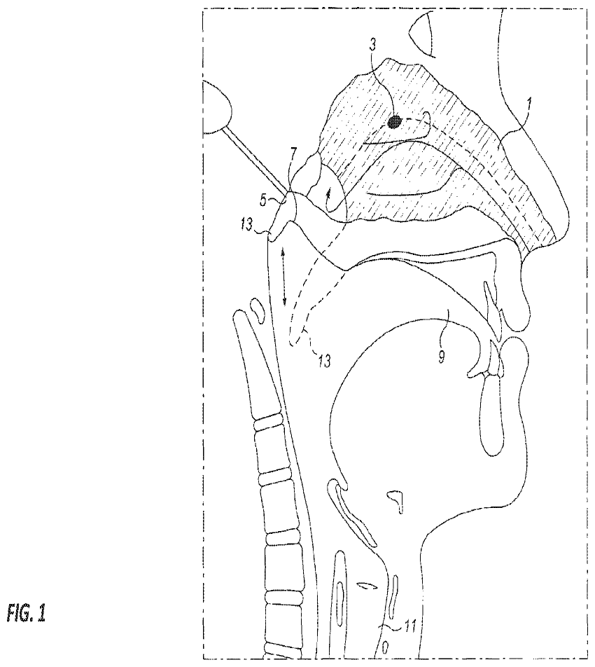

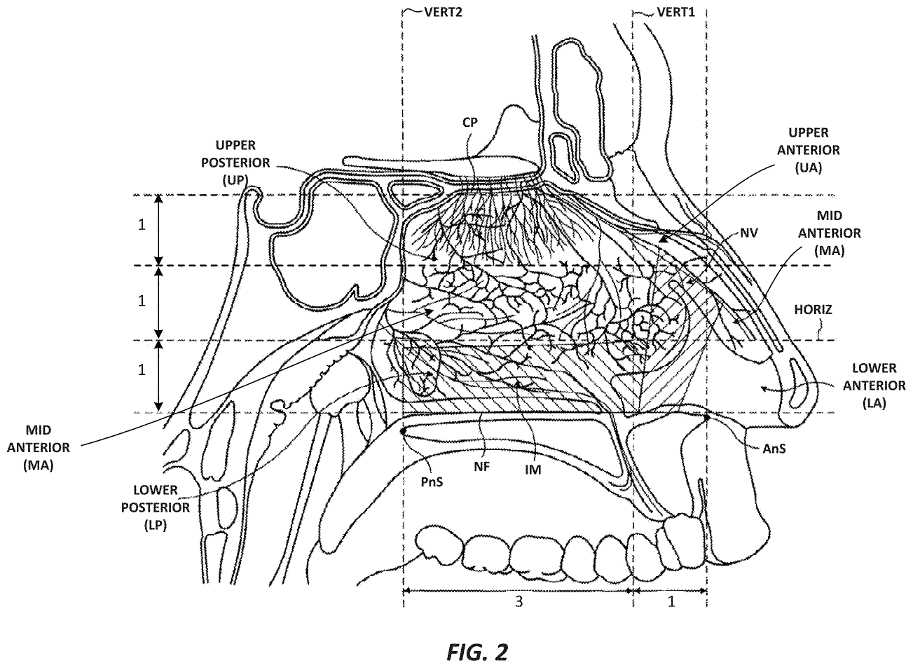

### DETAILED DESCRIPTION

As used herein, the terms “about” and “approximately,” when used to modify a numeric value or numeric range, indicate that deviations of up to 10% above and down to 10% below the value or range remain within the intended meaning of the recited value or range. In some embodiments, “about” refers to ±10%. In some embodiments, “about” refers to ±9%. In some embodiments, “about” refers to ±8%. In some embodiments, “about” refers to ±7%. In some embodiments, “about” refers to ±6%. In some embodiments, “about” refers to ±5%. In some embodiments, “about” refers to ±4%. In some embodiments, “about” refers to ±3%. In some embodiments, “about” refers to ±2%. In some embodiments, “about” refers to ±1%. It is understood that wherever aspects are described herein with the language “about” or “approximately” a numeric value or range, otherwise analogous aspects referring to the specific numeric value or range (without “about”) are also provided. It is also understood that wherever aspects are described herein referring to a numeric value or range without the language “about” or “approximately,” otherwise analogous aspects referring to “about” or “approximately” the specific numeric value or range are also provided.

As used herein, the term “chronic” refers to the persistence of a condition or disease for 12 weeks or longer, with or without acute exacerbations.

As used herein, the term “chronic sinusitis,” “CS,” chronic rhinosinusitis “CRS,” and “chronic rhinosinusitis without nasal polyps” (CRSsNP) are used synonymously in the present disclosure and are generally used interchangeably in the field to emphasize the condition associated with an anatomic finding of chronic inflammation inside the sinuses (irrespective of nasal pathology), while the terms “nasal polyps” (NP) and “chronic rhinosinusitis with nasal polyps” (CRSwNP) are used synonymously and are intended to refer to the presence of polyps in the nasal cavities (irrespective of sinus pathology), recognizing that these conditions can be overlapping, exist on a continuum, and can be accompanied by some degree of diffuse sino-nasal inflammation.

Reference will now be made in detail to the exemplary embodiments of the disclosure, examples of which are illustrated in the accompanying drawings. Wherever possible, the same reference numbers will be used throughout the drawings to refer to the same or like parts.

Delivery Devices

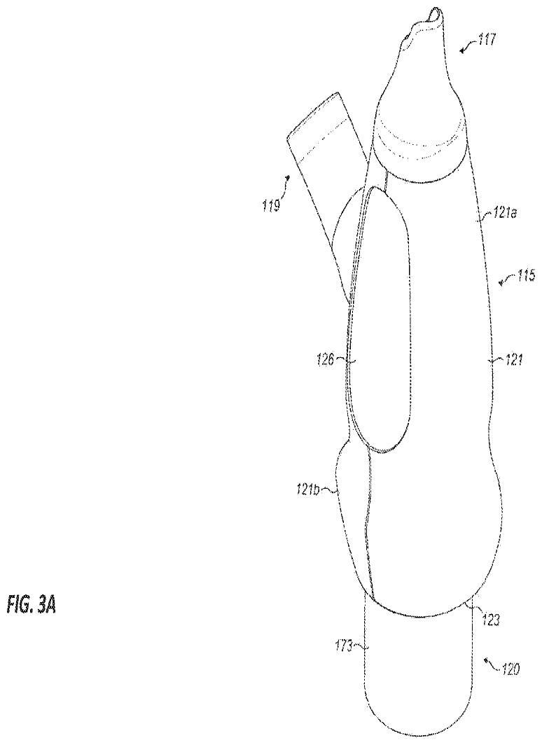

Suitable exhalation delivery devices, including the device disclosed herein, are disclosed in U.S. Pat. Nos. 11,033,696, 11,554,229, and WO-A-2000/051672, the contents of each of which are herein incorporated by reference in their entirety. In some embodiments, as depicted in FIGS. 3A and 3B, the delivery device comprises a housing 115, a nosepiece 117 for fitting in a nasal cavity of a subject, a mouthpiece 119 into which the subject in use exhales, such as to enable delivery of a gas flow, e.g., exhalation breath, into and through the nasal airway of the subject on exhalation by the subject through the mouthpiece 119, and a delivery unit 120, which is manually actuatable to deliver substance to the nasal cavity of the subject.

In some embodiments, the housing 115 comprises a body member 121, comprised of a substantially elongate, tubular section which includes an aperture 123 at one end thereof, through which projects an actuating part of the delivery unit 120, in this embodiment, as defined by the base of a substance-containing chamber 173 of a substance-supply unit 169. In some embodiments, the body member 121 comprises two body sections 121a, b which are fixed together. In some embodiments, the body sections 121a, b include inter-engaging lugs 124 and detents 125, here of snap-fit type, and sealing elements 126, which act to close the gas flow, e.g., exhalation breath, paths at the junctions of the body sections 121a, b. In some embodiments, the sealing elements 126 are adhesively bonded, but could alternatively be mechanically bonded, such as by welding. In some embodiments, the sealing elements 126 could be omitted.

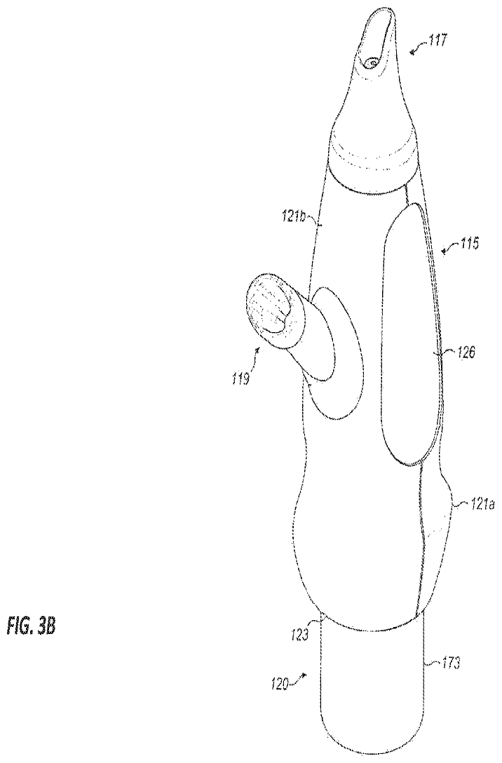

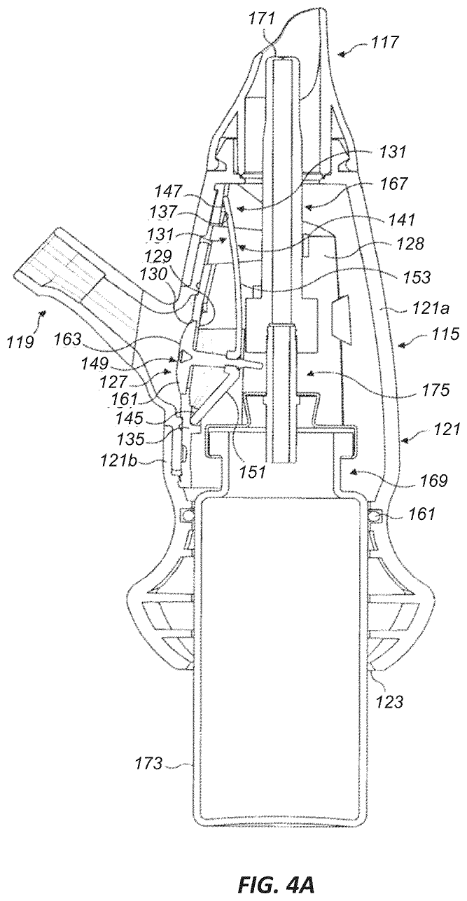

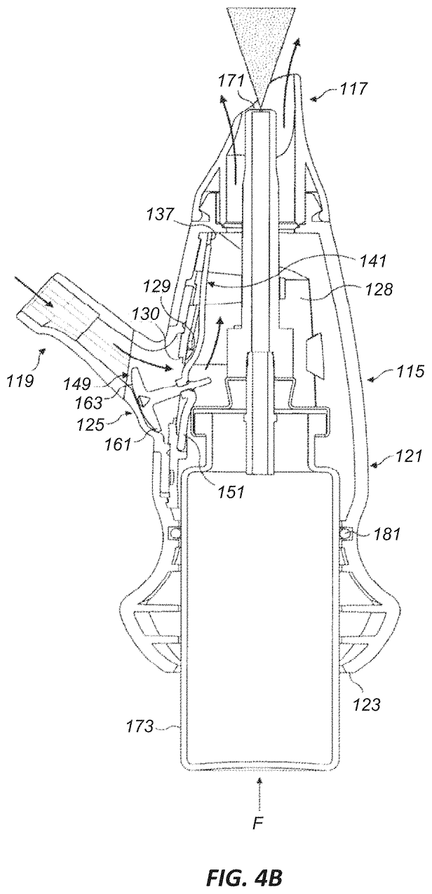

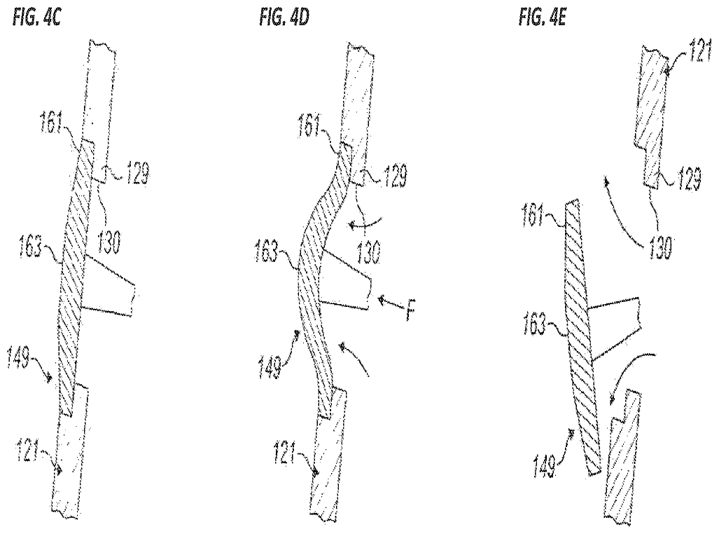

In some embodiments, the housing 115 further comprises a valve assembly 127 which is fluidly connected to the nosepiece 117 and the mouthpiece 119, and operable between closed and open configurations, as illustrated in FIGS. 4A and 4B, such as to provide for gas flow, e.g., exhalation breath flow, through the nosepiece 117 simultaneously with actuation of the delivery unit 120, as will be described in more detail hereinbelow. In particular, in some embodiments the device generally and valve assembly specifically are configured to provide a burst effect of gas flow, e.g., exhalation breath. For example, the valve assembly may be configured to open and release an initial gas flow, e.g., exhalation breath, having an initial pressure and flow rate that is higher than the pressure and flow rate of the remaining gas flow, e.g., exhalation breath. That is, the valve assembly may be configured to provide a gas flow, e.g., exhalation breath having an initial transient pressure and flow rate that is higher than a steady state pressure and flow rate.

The valve assembly 127 comprises a main, body element 128 which includes a valve seat 129 defining a valve opening 130, and a valve element 131 which is movably disposed to the body element 128 between closed and open positions, as illustrated in FIGS. 3A-3B and 4A-4E. As particularly illustrated in FIG. 4A, in some embodiments. the body element 128 comprises a pivot 135 to one, lower side of the valve seat 129, to which one end 145 of the valve element 131 is pivoted, and a sliding surface 137 to the other, upper side of the valve seat 129, against which the other end 147 of the valve element 131 is slidable.

In some embodiments, the valve element 131 comprises an elongate arm 141 that is a flexible arm at one end 145, the lower end of which is pivoted to the pivot 135 of the body element 128, and the other, upper end 147 of which slideably engages the sliding surface 137 of the body element 128, and a valve member 149 which is supported by the arm 141. In some embodiments, the arm 141 comprises a first, here lower, arm section 151, which is biased, here inwardly, such that, when the valve element 131 is in the closed, rest position, the lower arm section 151 is inclined inwardly relative to the longitudinal axis of the housing 115 and engageable by the substance-supply unit 169 when manually actuated to move the valve element 131 to the open position, as will be described in more detail hereinbelow.

In some embodiments, the arm 141 further comprises a second, here upper, arm section 153, which engages the sliding surface 137 of the body element 128 and acts to bias the valve element 131 to the closed position. In some embodiments, the valve member 149 comprises a seal 161, that, in some embodiments, is a flexible or resilient element, which acts to close the valve opening 130 as defined by the valve seat 129 when the valve element 131 is in the closed position, and a support 163 which supports a central region of the seal 161.

In some embodiments, and referring to FIGS. 4C to 4E, where the seal 161 is centrally supported, when the valve element 131 is moved to the open position, the support 163 biases the central region of the seal 161, as illustrated in FIG. 4D, causing the seal 161 to bulge outwardly in this central region and thus provide that the seal 161 engages the valve seat 129 only at the peripheral edge of the seal 161, until the point is reached when the seal 161 is suddenly and explosively released from the valve seat 129, as illustrated in FIG. 4E. This mode of release is believed to be particularly effective in the present disclosure where it is desired to achieve a sudden, initial burst of gas flow, e.g., exhalation breath, in that substantially the entire sealing surface of the seal 161 is released in one instant, which compares to an alternative mode of a peeling-type release, where a smaller section of a sealing surface is released, followed by the remainder of the sealing surface, which tends to provide a smaller initial burst pressure.

In some embodiments, the valve element 131 provides for a burst of gas flow, e.g., exhalation breath on opening thereof, having a first, initial burst phase followed by a second, extended burst phase, wherein the peak flow rate in the initial burst phase has a higher flow rate than the average flow rate in the extended burst phase, and the extended burst phase is of substantially greater duration than the initial burst phase. In some embodiments, the delivery unit 120 provides for delivery of substance subsequent to opening of the sealing member 149. In some embodiments, the delivery unit 120 comprises an outlet unit 167 for delivering substance into the nasal airway of the subject, and a substance-supply unit 169 for delivering substance to the outlet unit 167.

In some embodiments, the outlet unit 167 comprises a nozzle 171 for delivering substance to the nasal airway of the subject. In some embodiments, the nozzle 171 is configured to provide an aerosol spray. In some embodiments, for the delivery of a liquid, the nozzle 171 could be configured to deliver a liquid jet as a column of liquid. In some embodiments, the distal end of the outlet unit 167 is configured to extend at least about 2 cm, preferably at least about 3 cm, and more preferably from about 2 cm to about 3 cm, into the nasal cavity of the subject. In some embodiments, the substance supply unit 169 is a pump unit, which comprises a substance-containing chamber 173 which contains substance and extends from the aperture 123 in the housing 115 as the actuating part of the substance-supply unit 169, and a mechanical delivery pump 175 which is actuatable, here by depression of the substance-containing chamber 173, typically by a finger or thumb of the subject, to deliver a metered dose of substance from the substance-containing chamber 173 to the outlet unit 167 and from the nozzle 171 thereof, here as an aerosol spray.

In some embodiments, the substance-containing chamber 173, when depressed to actuate the substance supply unit 169, engages the lower arm section 151 of the arm 141 of the valve element 131, such as simultaneously to provide for actuation of the substance-supply unit 169 and opening of the seal 161 of the valve element 131, whereby substance, here in the form of a spray, and an gas flow, e.g., exhalation breath, here as a burst of air, are simultaneously delivered to the nasal cavity of the subject. In some embodiments, the mechanical delivery pump 175 is a liquid delivery pump for delivering a metered dose of substance, but, in some embodiments, the mechanical delivery pump 175 could be a powder delivery pump, which delivers metered doses of a powdered substance on actuation thereof. In some embodiments, the substance-supply unit 169 is a multi-dose unit for delivering a plurality of metered doses of substance in successive delivery operations. In some embodiments, the substance-supply unit 169 could be a single-dose unit for delivering a single metered dose of substance or a duo-dose unit for delivering two metered doses of substance in two successive delivery operations. In some embodiments, the substance-supply unit 169 could comprise a dry powder delivery unit which delivers metered doses of a substance, as a dry powder, on actuation thereof. In some embodiments, the substance-supply unit 169 could comprise a nebulizer which delivers metered doses of a substance, as an aerosol spray, on actuation thereof.

In some embodiments, the substance-supply unit 169 could comprise an aerosol canister for delivering metered volumes of a propellant, preferably a hydrofluoroalkane (HFA) propellant or the like, containing substance, either as a suspension or solution. In some embodiments, the housing 115 further comprises a sealing member 181, here an annular seal, in the form of an O-ring, which slideably receives the substance-containing chamber 173 of the substance-supply unit 169, such as to prevent the escape of the delivered gas flow, e.g., exhalation breath from the aperture 123 in the housing 115. In some embodiments, the sealing member 181 could be omitted.

In some embodiments, the exhalation delivery device described herein is used to deliver nasal steroids to a patient in need thereof. In some embodiments, the nasal steroid is fluticasone propionate. In some embodiments, this combination product is composed of a pharmaceutical industry standard amber glass vial containing a suspension of fluticasone propionate, a standard metering spray pump, and device that includes an asymmetrically shaped sealing nosepiece, valving mechanism, and flexible mouthpiece. The patient inserts the sealing nosepiece into one nostril, so it seals well with the flexible nasal tissues, and inserts the mouthpiece between the lips. The nosepiece's asymmetrical shape serves to stent the nasal valve, disproportionately widening the narrow superior aspect, while preventing obstruction of the opening by compressed soft tissues and, with the mouthpiece in the mouth, provides “2-point fixation” for device orientation. After taking a deep breath, the patient blows into the exhalation delivery device mouthpiece. Static positive pressure is created by a closed internal valve mechanism that is subsequently released in an “air burst” when the spray pump is actuated by pressing the vial. The device is designed to coordinate release of the drug into the “burst” of orally generated pressure/airflow, and not before, and to reduce demands on the patient for coordination during use (as required during use of many orally inhaled medications). Exhalation through the mouth against the device resistance also causes the soft palate to elevate and create an airtight seal, separating the oral cavity from the nasal cavities. A nasal spray applicator with swirl chamber extends from the metering pump to the tip of the nosepiece. Upon actuation, but not before, the exhalation delivery device allows air to be channeled from the mouthpiece to the sealing nosepiece. Under these conditions, exhaled air creates a positive intranasal pressure which acts to expand spaces that are narrowed by soft tissue or inflammation. This is the opposite of the negative pressure created by Bernoulli's forces during “sniffing” of a standard-delivery inhaled nasal spray, which tend to further narrow or collapse spaces that are narrowed by soft tissues or inflammation. The exhaled breath, conditioned to physiologic temperature and humidity from the lungs, accompanies the aerosol expelled by the spray pump applicator beyond the nasal valve and creates “Bi-Directional” airflow that floats medication beyond the head of the inferior turbinate and into upper posterior region of the nasal cavity above the inferior turbinate, including the middle meatus and ostiomeatal complex. Because the exhaled intranasal pressure is proportional to the pressure in the oral cavity, and slightly less due to pressure drop across the device, the intranasal pressure is balanced with the intraoral pressure, resulting in a patient communication posterior to the nasal septum, and allowing pressure to be relieved without building up in the nasal passages or Eustachian tubes and for air to ultimately exit through the contralateral nostril. The biomechanics produced by the exhalation delivery device during use are intended to deliver topically-acting anti-inflammatory into the region of the nasal cavity where the paranasal sinuses ventilate and drain. In addition, the action of the device is intended to produce other effects that may improve sinonasal symptoms, such as changes in carbon dioxide, which is present in the exhaled breath delivered into the nose through the device, that may in turn influence inflammatory mediator activity and neuropeptide activity, and by actions such as removal of nitric oxide, change in pH, or positive pressure.

Fluticasone propionate, a corticosteroid, having the chemical name S-(fluoromethyl) 6α,9-difluoro-11β,17-dihydroxy-16α-methyl-3oxoandrosta-1,4-diene-17β-carbothioate, 17-propionate and the following chemical structure:

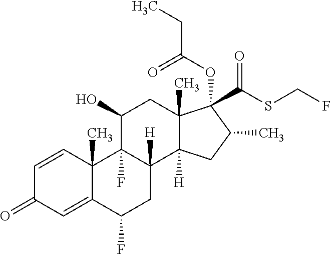

Fluticasone propionate is a white powder with a molecular weight of 500.57, and the empirical formula is C₂₅H₃₁F₃O₅S. It is practically insoluble in water, freely soluble in dimethylformamide, sparingly soluble in acetone and dichloromethane, and slightly soluble in 96% ethanol.

In some embodiments, the fluticasone propionate nasal spray (93 μg-metered doses) for nasal administration is used with a delivery device (e.g., an exhalation delivery system) that delivers an aqueous suspension of microfine fluticasone propionate having a particle size distribution in the range of 0 to 5 microns for topical nasal administration by means of a metering, atomizing spray pump and exhaled breath. In some embodiments, the delivery device also comprises microcrystalline cellulose and carboxymethylcellulose sodium, dextrose, benzalkonium chloride, polysorbate 80, edetate disodium dihydrate, sodium hydroxide and hydrochloric acid (to adjust pH), and purified water, and has a pH between 5 and 7.

The delivery device is designed to create closed-palate bi-directional biomechanics during use that distribute medication into posterior/superior sites in the nasal cavity, particularly areas posterior to the nasal valve and superior to the inferior turbinate, including the OMC as well as into sinus cavities that have been surgically opened. By delivering a potent topically acting steroid with anti-inflammatory activity broadly and in superior/posterior segments of the nose and into the sinus cavities, inflamed tissue associated with CS can be addressed in important target regions for treatment of CS, including at the sinus ostia where ventilation and drainage of the paranasal sinuses normally occurs, and inside the sinus cavities themselves in patients with or without surgically opened sinuses. This deposition pattern supports treatment of symptoms caused by inflammation specific to these deep intra sinonasal regions. Additionally, this deposition pattern offers the potential to reduce edema and inflamed tissue that may be blocking the ostia and preventing the natural ventilation and mucociliary drainage from the sinuses into the nasal cavity.

In embodiments, the delivery device is used to deliver a total daily dose of 372 or 744 μg of fluticasone propionate. In some embodiments, fluticasone propionate is metered to deliver 93 μg per spray. In some embodiments, the fluticasone propionate is delivered via 1 spray in each nostril, twice daily. In some embodiments, the fluticasone propionate is delivered via 2 sprays in each nostril, twice daily. In some embodiments, the fluticasone propionate is delivered via 1 or 2 sprays in each nostril, twice daily. In some embodiments, the fluticasone propionate is administered at regular intervals. For example, fluticasone propionate may be administered via the delivery system in the morning and in the evening.

In some embodiments, the present disclosure provides methods for treating a subject having sinus and/or nasal inflammatory disease such as sinusitis or rhinosinusitis. In some embodiments, the sinusitis or rhinosinusitis is chronic rhinosinusitis. In some embodiments, the sinusitis or rhinosinusitis is chronic rhinosinusitis with nasal polyps. In other embodiments, the sinusitis or rhinosinusitis is chronic rhinosinusitis without nasal polyps.

The present disclosure will now be described herein with reference to the following non-limiting Examples.

### Example 1: Clinical Study Design

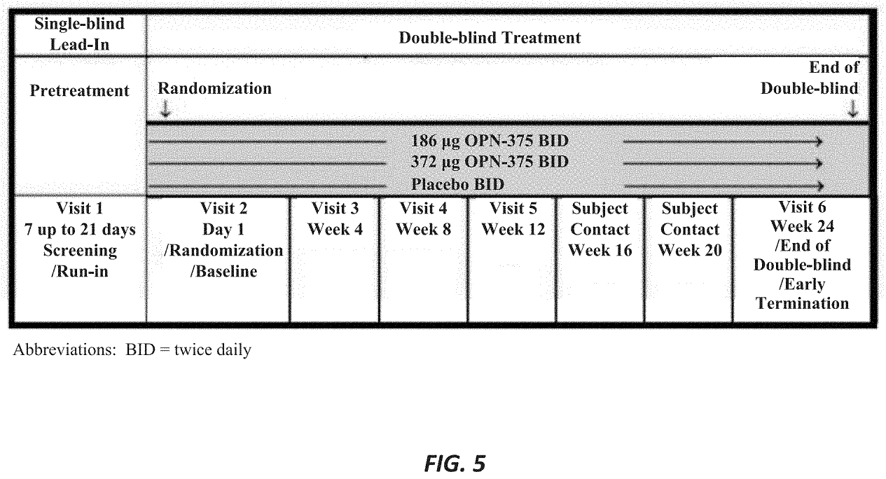

Two pivotal multi-center, multi-national studies in CS, Studies 3205 (Study A; CRS with and without nasal polyps) and 3206 (Study B; CRS without nasal polyps), had common study objectives and generally similar design. As previously noted, sinusitis is a symptomatic inflammation of the paranasal sinuses and nasal cavity. If the duration of sinusitis is more than 12 weeks, with or without acute exacerbations, it is deemed to be “chronic rhinosinusitis” (CRS) (Fokkens et al., 2020; Orlandi et al., 2021). CS and CRS are generally treated as synonyms and refer to patients with chronic symptomatic sinus and/or nasal inflammation. All enrolled subjects in both pivotal studies had to have CT-scan proven evidence of disease inside the sinus cavities. The primary design distinction between these studies was that Study A allowed for inclusion of CS patients either with (CRSwNP) or without NP (CRSsNP) and Study B included only subjects with CS who did not have polyps in the nasal cavity (CRSsNP). Both studies were adequate and well controlled, with a primary objective to evaluate the efficacy and safety of 2 doses of OPN-375 (exhalation delivery device for administering fluticasone propionate) compared with placebo. After a 7- to up to 21-day single-blind placebo eligibility verification period, subjects were randomly assigned (1:1:1) to receive placebo or OPN-375 at doses of 186 μg or 372 μg BID for 24 weeks. The overall duration of study participation for subjects was approximately 28 weeks. An overview of the study design is schematically presented in FIG. 5.

The eligibility criteria were designed to ensure a suitably generalizable population of patients with CS that had adequate severity of disease to allow for improvement with treatment to be detected, and the criteria were also representative of a broad population of patients with CS who were medically stable enough and otherwise suitable for participation in a 6-month study.

Patients who met initial eligibility criteria at Screening entered a 7- to 21-day single-blind placebo period to determine whether the patient met remaining eligibility criteria and to ensure that they could comply with study procedures. At Screening, patients had nasoendoscopy-related procedures (nasal examination and assessment of nasal cavity, including evaluation of edema, polyps, or mucopurulent discharge) performed. During the single-blind eligibility verification period, patients administered morning (AM) and evening (PM) doses of placebo and completed a daily diary (electronic) immediately prior to each of these doses to record instantaneous (evaluation of symptom severity immediately preceding the time of scoring) and reflective (evaluation of symptom severity over the past 12 hours) scores for nasal symptoms.

At the end of the single-blind placebo eligibility verification period, eligible patients underwent a CT scan of the sinuses to confirm CT eligibility criteria and establish a baseline assessment of sinus opacification. Patients meeting eligibility criteria at this time (including symptom severity and sinus opacification) were randomly assigned to receive 1 of 3 treatments: 186 or 372 μg of OPN-375 or placebo twice daily (BID) for the 24-week double-blind treatment phase. Patients in the placebo group received a placebo substance delivered with the exhalation delivery device as herein described.

Randomization of patients was stratified by presence of NP of grade 1 or larger at baseline (NP Present vs Absent, Study A alone) and previous sinus surgery (Yes vs No) (previous sinus surgery was defined as evidence of ethmoidectomy [partial or total], or maxillary antrostomy). The continuous variables of subject age, weight (kg), height (cm), and body mass index (BMI) (kg/m²), and the categorical variables of gender, ethnicity, and race were summarized using descriptive statistics. Subject characteristics of nasal polyp status at baseline and prior sinus surgery status were summarized descriptively.

In Studies A and B, the following safety parameters were assessed during the double-blind treatment phase: adverse events (AEs), nasal/sinus endoscopic examinations (not simply examination by nasal speculum), laboratory evaluations, ocular examinations, vital signs assessment, and concomitant medication use. Due to the challenges associated with the coronavirus disease 2019 (COVID-19) pandemic, the number of nasal examinations performed during the study was reduced (performed during the placebo run-in period and at the Week 24/early termination [ET] visit only). In addition, the requirement for ocular examinations, originally required at baseline and end of study (EOS) visits, was removed during the acute phases of the COVID-19 pandemic because of the difficulties in obtaining eye examinations and in light of the pre-existing body of relevant safety data. Subjects were allowed to delay the Week 24 Visit in case of COVID-19 or any upper respiratory infection and continue taking study drug until they could have the Week 24 Visit. Remote visits were allowed as needed.

An AE was defined as any untoward medical occurrence associated with the use of an investigational product in humans, whether or not considered related to the investigational product. This included any occurrence that was new in onset or aggravated in severity or frequency from the baseline condition, with the exception of changes or worsening of symptoms of chronic sinusitis. A treatment-emergent adverse event (TEAE) was defined as an AE with onset date on or after the first dose of randomized double-blind study drug. Clinically significant findings per the investigator from unscheduled ocular examinations were reported as AEs.

In Studies A and B, in addition to spontaneously reported AEs, certain clinical events, such as epistaxis, septal erosion/perforation, erosion/ulceration located in an area other than at the septum, and mucosal candidiasis, that were more intensively evaluated via nasal endoscopic examination, were recorded as either medical history/baseline findings (pretreatment nasal examination) or AEs if deemed to be clinically significant by the investigator starting at Visit 1 (Screening). In these studies, both active bleeding (inclusive of any evidence of recent blood in the nasal cavity, such as blood-tinged mucus; mild nasal bleeding, medical intervention not indicated; clinically evident nasal bleeding, medical intervention necessary]) and nonactive bleeding (i.e., evidence suggesting prior blood entry into the nasal cavity such as darker blood, appearing thick or “solid”, such as clots) were all recorded as “epistaxis” on the nasal examination case report form (CRF). Only those nasal examination findings deemed by the investigator to be clinically significant were recorded as an AE.

All AEs with an onset from the time the subject gave informed consent until the subject's final visit (either the scheduled end of study (EOS) or at an ET visit, if applicable) were recorded in the CRF. In both studies, the period of observation for collection of AEs extended from the time the subject gave informed consent until completion of the treatment period or an ET visit. AEs with onset dates on or after the first dose of randomized study drug were considered treatment emergent. The severity, date of occurrence, duration, frequency, treatment, and outcome were recorded along with classification as being serious or nonserious and the investigator's assessment whether the AE was study drug related (adverse drug reaction) or device related (adverse device reaction). Action taken regarding the study drug was also recorded (drug withdrawn, drug interrupted).

Symptoms of chronic sinusitis were collected as part of the primary and secondary endpoints. Changes or worsening of symptoms of chronic sinusitis were not to be reported as AEs. However, acute exacerbations of chronic sinusitis that resulted in an escalation in care (e.g., received an antibiotic, oral steroid, sought medical care for the event) were recorded as an AE. All events that met the definition of a serious adverse event (SAE) were to be reported as SAEs, regardless of whether they were protocol-specific assessments.

Due to the challenges associated with the COVID-19 pandemic, the number of nasal endoscopic examinations performed during the study was reduced (performed during the eligibility verification period prior to double-blind treatment and at the Week 24/ET visit only). The nasal examination was performed via nasoendoscopy (not simply by nasal speculum examination) during the placebo eligibility verification period and at Week 24 and was guided by a nasal examination worksheet provided by the sponsor. The worksheet was formatted as a series of questions with check boxes and fields for narrative text and designed to collect information about the conditions detailed below. The nasal examination worksheet was completed by the physician who performed the examination. The examiner assessed both sides of the nasal cavity and indicated their clinical judgment as to severity and relationship to study drug/device for each nasal finding. Location of the finding was also captured. If decongestants and/or local anesthetics were administered as preparation for the nasoendoscopy, these were also recorded on the form as well as on the concomitant medication CRF page. If decongestants and/or local anesthetics were administered for the initial (screening) nasoendoscopy, the same was required with each subsequent examination.

Nasoendoscopy was performed using a rigid or flexible endoscope; the same type and size endoscope was to be used throughout the study. The examiner must have been able to visualize the middle meatus with the scope.

At the nasal examination, the subject was evaluated for “epistaxis”, including non-active bleeding or other “epistaxis” observations requiring no treatment, septal erosion/ulceration/perforation, ulcerations in areas of the nose other than septum, and mucosal candidiasis. All findings were to be recorded on both the nasal examination CRF (nasal examination worksheet) and, if clinically significant, on the AE CRF beginning at Visit 1 (screening).

Ocular examinations were required at baseline and end-of-study visits when the studies were initiated; however, during the acute phases of the COVID-19 pandemic this requirement was removed by protocol amendment because of the difficulties in safely obtaining eye examinations. However, if any subject experienced an unexplained worsening in vision during the study, the protocol required the subject to have an ocular examination. An ocular examination by an examiner who is a health care provider with expertise in examination and diagnosis of conditions of the eye during an unscheduled visit was promptly performed if a subject experienced any unexplained worsening in vision during the study. If an ocular examination was done, the examiner completed a worksheet to capture visual acuity, intraocular pressure (IOP), and lens opacities. The worksheet included instructions for grading nuclear, cortical, and posterior sub-capsular (PSC) cataracts, if any cataracts were identified during the examination.

Any findings of cataract or clinically significant changes in IOP were reported as AEs. AEs of new onset PSC cataract were of particular interest due to the fact that spontaneous PSC cataracts are the least prevalent cataracts in the general population (nuclear and cortical are considered common), can be relatively rapidly progressing, and particularly because PSC cataracts are believed to be associated with exposure to corticosteroids.

Clinical laboratory tests (hematology, serum chemistry, urinalysis, urine drug screen, and urine pregnancy) were performed at screening (Visit 1) only. Urine pregnancy tests were conducted at protocol-specified intervals. A blood sample for severe acute respiratory syndrome coronavirus 2 (SARS-CoV-2) serology test at Visit 6/Week 24 was collected unless the subject declined testing. If any laboratory tests were performed as part of a subject's standard of care on or after day 1 and a change from the screening (Visit 1) laboratory test result was considered to be clinically significant, in the opinion of the investigator, it was considered an AE, if it met the definition for an AE. Significant abnormal values occurring during the study were followed until repeat test results returned to normal, stabilized, or were no longer clinically significant. Vital signs (blood pressure [BP] and pulse rate) were measured and recorded at each site visit after the subject had been at rest for at least 5 minutes. Concomitant medications included all medications and other treatments taken by a subject during the study, including those treatments initiated prior to the start of the study. On-treatment findings that were considered clinically significant by the investigator were reported as TEAEs. Below are the assessments used to measure efficacy of OPN-375 treatment in patients with CS for the selected endpoints. Endpoints are summarized in Table 1.

Nasal Symptoms: During the double-blind phase of both studies, electronic diaries were completed twice daily (AM and PM) by the subject to capture daily nasal symptoms (DNS) (i.e., symptom scores for nasal congestion, nasal discharge [anterior and/or posterior], facial pain or pressure, and loss of sense of smell). Both individual symptom and composite symptom scores (CSS) were derived from these daily recordings up through Week 12 of the double-blind treatment phase. The CSS is the sum of the scores for the 3 nasal symptoms (nasal congestion, facial pain or pressure sensation, and nasal discharge [anterior and/or posterior]) and ranges from 0 to 9 for any given study day/time and type of measure (instantaneous vs reflective).

Computed tomography (CT) scans: CT scan of the sinuses (at baseline and Week 24/Early Termination [ET]) were used to assess structural changes in the sinuses. The quantitative variables were sinus volume (the region of interest defined by radiologist review and mapping of each defined sinus space), opacified volume (space within the region of interest that was not air or bone, defined by Hounsfield unit signal intensity), and percentage of opacified volume (POV) in the each of the maxillary and ethmoid sinuses. The average of the percentages of opacified volume (APOV) across the ethmoid and maxillary sinuses was calculated and used as the study measure of intra-sinus inflammation.

Acute exacerbations of CRS (AECRS): AECRS were defined as an event characterized by 1) acute worsening of one or more of the cardinal diagnostic symptoms of CS (facial pain or pressure, nasal congestion/blockage, rhinorrhea, and/or reduction in sense of smell), lasting at least 3 days, that causes the subject to seek medical care that was also accompanied by 2) escalation of care, defined as either initiation of antibiotics or oral steroids, or the escalation of treatment involving an unscheduled acute care visit (e.g., emergency room [ER] or acute care outpatient clinic) or inpatient care for increased symptoms of CS or for acute sinusitis.

Sinonasal Outcome Test 22-item version (SNOT-22): The SNOT-22 is a subject-completed questionnaire which was used to measure symptom severity, quality of life, and effect on patient function. Each of 22 items is rated from 0 (no problem) to 5 (problem as bad as it can be), with a possible SNOT-22 total score ranging from 0 to 110. The minimum clinically important difference (MCID) is 8.9 points (Soler et al., 2018).

Pittsburgh Sleep Quality Index (PSQI): The PSQI is a validated, self-rated questionnaire which was used to assess sleep quality and disturbances over the past month. Nineteen individual items scored 0 (no difficulty) to 3 (severe difficulty) generate 7 component scores: subjective sleep quality, sleep latency, sleep duration, habitual sleep efficiency, sleep disturbances, use of sleeping medication, and daytime dysfunction. The sum of these scores yields 1 global score ranging 0 to 21, with higher scores indicating worse sleep quality. Scores of >5 is associated with poor sleep quality.

Patient Global Impression of Change (PGIC): The PGIC is a self-report rating of the patient's perceived change in symptoms using a balanced Likert scale from 1 (very much improved) to 7 (very much worse). Subjects with PGIC scores of 1, 2, or 3 were considered improved. Subjects with missing values were considered not improved. The PGIC is a good measure for assessing the clinical relevance of treatment because it is a global and directly patient-reported measure of whether the subject feels that he/she has improved (or worsened) with treatment.

Additional assessments of efficacy which were conducted in the 2 pivotal studies but for which their respective other secondary endpoints are not presented in this document included Lund-Mackay (LM) Staging and Zinreich Modification of the Lund-Mackay Staging (ZLM) of the CT scans, nasal endoscopy for NP grading and subject-completed questionnaires to measure health-related quality of life (EuroQol-5 dimensions [EQ-5D], 36-Item Short Form Health Survey version 2 [SF-36v2], and Short-Form Six-Dimension [SF-6D]), and depressive symptoms (Quick Inventory of Depressive Symptomatology [QIDS]). A Smell Identification Test (SIT) was also performed as an objective measure. Subjective (patient reported) and objective (using RAND criteria) eligibility for surgery were also assessed at baseline and follow-up as a measure of treatment efficacy. Health economic information related to CS was collected using the Health and Work Performance Questionnaire (HPQ). Detailed descriptions of these assessments as well as results of analyses are presented in the individual clinical study reports (CSRs) for Study A and Study B.

The co-primary outcomes in each of the 2 pivotal studies in patients with CS were symptoms, measured by the CSS, and sinus opacification, measured by the APOV in the ethmoid and maxillary sinuses. The co-primary endpoints, key secondary and the other secondary efficacy endpoints for which data are presented in this document are listed in Table 1.

TABLE 1
Efficacy Endpoints in Clinical Studies.

| | | | |
|---|---|---|---|
| | | Individual | |
| | | Pivotal | |
| | | Study | Integrated |
| Assessment | Endpoint | Summaries | Analysis |

Co-primary

| | | | |
|---|---|---|---|
| Symptoms | Change from baseline to the end | √ | √ |
| (CSS) | of Week 4 in average total | | |
| | instantaneous AM CSS | | |
| | (instantaneous: evaluation of | | |
| | symptom severity immediately | | |
| | preceding the time of scoring) of: | | |
| | nasal congestion, facial pain or | | |
| | pressure sensation, and nasal | | |
| | discharge (anterior and/or | | |
| | posterior). | | |
| Sinus | Change from baseline to Week | √ | √ |
| inflammation | 24/ET in the APOV in the | | |
| (CT-scan) | ethmoid and maxillary sinuses. | | |

Key Secondary (Type-1 Error Controlled Analyses of Pooled Data)

| | | | |
|---|---|---|---|
| Symptoms | Change from baseline to Week | — | √ |
| (CSS) in | 4—in CSS in subjects who were | | |
| patients | symptomatic despite use of a | | |
| entering study | standard nasal steroid spray (e.g., | | |
| using a | Flonase ®, Nasonex ®) for | | |
| standard- | treatment of CS at study entry | | |
| delivery nasal | | | |
| steroid | | | |
| Exacerbations | Frequency of acute exacerbations | — | √ |
| | of CS over the 24-week treatment | | |
| | period, defined as a worsening of | | |
| | symptoms that required an | | |
| | escalation of treatment | | |

Other Secondary

| | | | |
|---|---|---|---|
| CSS over time | Change from baseline in CSS | √ | √ |
| | (includes congestion, facial pain | | |
| | or pressure sensation, and nasal | | |
| | discharge [anterior and/or | | |
| | posterior]) at the end of Weeks 8 | | |
| | and 12 | | |
| DNS | Change from baseline to Weeks | √ | √ |
| | 4, 8, and 12 on the individual | | |
| | symptoms of facial pain or | | |
| | pressure sensation, nasal | | |
| | discharge (anterior and/or | | |
| | posterior), nasal congestion, and | | |
| | sense of smell | | |
| | Note: For Study B, individual | | |
| | symptom data to Week 4 was an | | |
| | additional key secondary | | |
| SNOT-22 | Change from baseline to Weeks | √ | — |
| | 4, 8, 12, and 24/ET in disease- | | |
| | specific quality of life, symptoms | | |
| | and functioning as measured by | | |
| | the SNOT-22 total and | | |
| | subdomain scores (analyzed in | | |
| | each pivotal study) | | |
| | Change from baseline to Week | — | √ |
| | 24/ET in SNOT-22 total score in | | |
| | subjects who entered the study | | |
| | using a standard-delivery nasal | | |
| | steroid | | |
| Time-to-First | Time to first acute exacerbation | √ | — |
| Acute | of chronic sinusitis, defined as a | | |
| Exacerbation | worsening of symptoms that | | |
| | requires escalation of treatment | | |
| | Note: For Study B this was an | | |
| | additional key secondary | | |
| PSQI | Change from baseline to Week | √ | √ |
| | 24/ET in PSQI global and | | |
| | component scores (also at Week | | |
| | 12) | | |
| | Note: PSQI global score alone | | |
| | was analyzed for the pooled data | | |
| PGIC | Percent of subjects indicating | √ | √ |
| | improvement on the PGIC at | | |
| | Week 24/ET (also at Week 4) | | |
| | Note: Analysis of pooled data | | |
| | PGIC was used to anchor the CT- | | |
| | scan (APOV) magnitude of | | |
| | change to clinically important | | |
| | change | | |

Abbreviations:
AM = morning;
APOV = average of the percentages of opacified volume in ethmoid and maxillary sinuses;
CS = chronic sinusitis;
CSS = composite symptom score;
CT = computed tomography;
DNS = daily nasal symptoms;
ET = early termination;
PGIC = Patient Global Impression of Change;
PSQI = Pittsburg Sleep Quality Index;
SNOT-22 = Sinonasal Outcome Test 22-item version

The Statistical Analysis Plan (SAP) for Study A, which outlines a hierarchal testing strategy controlling for multiplicity and Type 1 error is considered the reference SAP for testing pooled endpoints because it was finalized prior to unblinding of data from either of the 2 studies included in the pooled analysis. Of note, in addition to the primary estimand, 2 supplementary estimands bearing relevance to anticipated real-world usage are presented herein: the alternative primary estimand which includes antibiotic use for CRS exacerbation, and the supplementary estimand 2 (SE-2) (defined in Table 2).

Populations for Analysis

Study A primary efficacy analyses evaluated the overall population of chronic sinusitis patients (both those with and without polyps in the nasal cavity), and additional analyses were performed for the CRSwNP and CRSsNP phenotypes. All chronic sinusitis subjects enrolled in Study B did not have nasal polyps; thus, efficacy was evaluated for only the CRSsNP phenotype.

It was also noted that, for the primary analyses of the overall population, in the event a subject was incorrectly stratified to the polyp or non-polyp strata or the previous surgical or non-surgical strata, they were analyzed as randomized (stratified), consistent with the intent-to-treat (ITT) principle. Subgroups, however, were defined based on a subject's actual polyp status and actual nasal surgery status at baseline.

For efficacy analyses of pooled (i.e., integrated) data, 3 populations were defined: (1) A primary analysis of the overall chronic sinusitis population, including all subjects in the Full Analysis Set (FAS) from both pivotal studies (a pooled population); and additional analyses limited to the two visual phenotypes based on presence or absence of polyps in the nasal cavity at baseline: (2) A CRSsNP Population which included chronic sinusitis subjects from both pivotal studies (a pooled population) without NP visualized at baseline. (3) A CRSwNP Population which included chronic sinusitis subjects from both pivotal studies (a pooled population) with NP visualized at baseline.

It was noted that, although Study B permitted enrollment of only CRS subjects without nasal polyps, 1 subject (081410) in the 372 μg OPN-375 group of Study B had a protocol violation of “presence of nasal polyps”. Applicable data for this subject up to the point of study discontinuation was included for the integrated analyses of the CRSwNP population.

Analyses of Co-Primary Endpoints

For the composite symptom score (CSS) co-primary endpoint, the estimator was the mean difference between treatments (active vs placebo) in change from baseline through the end of Week 4 in the 7-day average total instantaneous AM CSS. Estimation was based on a mixed model for repeated measures (MMRM) in which changes from baseline to Weeks 2 and 4 in the 7-day average total instantaneous AM CSS, respectively, were the repeated measures. The MMRM model employed restricted maximum likelihood (REML) for parameter estimation and the Kenward-Roger method for calculating the denominator degrees of freedom. An unstructured covariance matrix was used to estimate within-subject error.

The MMRM model included categorical effects for previous sinus surgery (Yes, No), NP status (present, absent), treatment (186 μg OPN-375, 372 μg OPN-375, placebo), study week (2, 4), treatment-by-day interaction, and the continuous covariate baseline 7-day average total instantaneous AM CSS, with baseline-by-day interaction. The model-based least squares (LS) mean difference between each active treatment group and placebo (active minus placebo), 95% confidence interval (CI), and p-value were displayed.

CT scans of the sinuses (at baseline and Week 24/ET) were used to assess objective changes in the sinuses. The quantitative variables were sinus volume (the region of interest defined by radiologist review and mapping of each defined sinus space), opacified volume (space within the region of interest that was not air or bone, defined by Hounsfield unit signal intensity), and percentage of opacified volume (POV) in the each of the maxillary and ethmoid sinuses; all of these were calculated throughout the use of a computer program where each series of 2-dimensional images into variables representing a 3-dimensional space were integrated. Higher POVs reflected greater inflammatory burden in the sinus cavity. These volumes were calculated per sinus and APOV was the average of the POV across the 4 sinus regions of interest (right and left maxillary sinuses and left and right ethmoid sinuses).

For the average of the percentages of opacified volume (APOV) co-primary endpoint, the estimator was the mean difference between treatments (active vs placebo) in the change from baseline to Week 24/ET APOV. Estimation was based on an analysis of covariance (ANCOVA) model including categorical effects for previous sinus surgery (Yes, No), NP status (present, absent), treatment (186 μg OPN-375, 372 μg OPN-375, or placebo), and baseline APOV. For integrated analyses of coprimary endpoints, a categorical term for protocol was included in the statistical model.

The primary efficacy estimands were the differences between active treatment (including each dose level of OPN-375) and placebo for each of the co-primary endpoints. The potential intercurrent events were: 1) use of systemic corticosteroids to treat CRS exacerbation or worsening of CRS nasal/sinus symptoms; 2) nasal surgery; and 3) discontinuation from study treatment. Poor scores on the CSS (score of 9) and APOV (100% if baseline APOV≥75%, or baseline+25% if baseline APOV<75%) were assigned at the time of initiation of systemic corticosteroids or nasal surgery. Missing CSS data following treatment discontinuation were estimated under the assumption that the missing data were missing at random (MAR) and that early discontinuation from the study (prior to Week 4) was unlikely to reflect a treatment effect. Missing APOV data due to study treatment discontinuation were imputed with a pattern mixture model (PMM) multiple imputation (Ml) using the Jump-to-Control (J2C) method which assumed that the missing data at Week 24/ET on the control arm (placebo) was MAR, and the missing data on the active treatment arms had the profile of the control arm at Week 24/ET.

Planned sensitivity analyses for the co-primary endpoints for each of the individual pivotal studies included 1) analyses that varied in how they handled intercurrent events and treatment discontinuations (see Table 2) and 2) tipping point analyses to determine the smallest change in imputation of missing values that would turn a statistically significant result in favor of active treatment to a result that was not statistically significant. In addition, there was a pre-planned analysis using ranked APOV values and analyses of both co-primary endpoints in the per-protocol set (PPS).

This primary estimand uses a composite strategy for subjects with treatment failure, assigning the same poor scores; the only difference is that the definition of treatment failure is expanded to encompass not only surgery or use of systemic corticosteroids (SCS) for disease worsening or exacerbation but also use of antibiotics for an exacerbation.

Tables 2 and 3 provide an overview of the estimands applied for analyses of endpoints as discussed herein.

TABLE 2
Primary and Supplemental Estimands.

| | Intercurrent Events | | |
|---|---|---|---|
| | Systemic | Nasal | Treatment/Study |
| Estimand | Corticosteroids | Surgery | Discontinuation |
| Primary | SCS for CRS: Composite | Composite | Treatment Policy |
| | Strategy¹; Poor Score² | Strategy; Poor | Strategy⁴ |
| | | Score | |
| Primary—Alternative | SCS for CRS: Composite | Composite | Treatment Policy |
| IE includes antibiotic use for | Strategy¹; Poor Score² | Strategy; Poor | Strategy |
| acute CRS exacerbations in | Antibiotic use: | Score | |
| addition to SCS for CRS. | Composite Strategy; | | |
| | Poor Score² | | |
| Supplementary—S1 | Treatment Policy | Composite | Composite Strategy; |
| | Strategy | Strategy; Poor | Poor Score |
| | | Score | |

Additional Sensitivity

| | | | |
|---|---|---|---|
| Supplementary—SE-2 | Treatment Policy | Composite | Treatment Policy |
| | Strategy | Strategy: Worst | Strategy |
| | | Score⁵ | |
| Supplementary—SE-3 | Composite Strategy; | Composite | Treatment Policy |
| | Poor Score | Strategy; Poor | Strategy |
| | | Score | |
| Supplementary—SE-4 | Composite Strategy; | Composite | Treatment Policy |
| | Worst Score | Strategy; Worst | Strategy |
| | | Score | |

Abbreviations:
APOV = average of the percentages of opacified volume;
CSS = composite symptom score;
CRS = chronic rhinosinusitis;
IE = intercurrent event;
SCS = systemic corticosteroids;
SE = supplementary estimand
¹Systemic corticosteroids use for CRS exacerbation or worsening of CRS nasal/sinus symptoms: Composite Strategy Systemic corticosteroid use for other indications is defined as treatment and study discontinuation event. Modified Treatment Policy Strategy: Subject continues in the study; if an IE for systemic steroids for CRS or nasal surgery occurs, a poor score is assigned as defined in (4); otherwise, treatment policy strategy is followed as defined in (6)
²Poor Scores: For CSS, a value of “9” is assigned. For APOV, a poor score is defined as follows: for a subject with a baseline APOV ≥75%, a value of “100%” is assigned; if baseline APOV <75%, a poor score is defined as baseline APOV + 25%
³Worst Scores: For CSS, a value of “9” is assigned. For APOV, a value of “100%” is assigned
⁴Treatment Policy Strategy; subjects may continue in the study until one of the following two mutually exclusive outcomes: a) Subject completes Week 4, use CSS endpoint value; completes Week 24/ET CT scan, use APOV endpoint value; b) Subject discontinues the study. Missing data due to study discontinuation following treatment discontinuation is imputed.

TABLE 3
Endpoints by Estimand.

| | | | |
|---|---|---|---|
| | | Alternative | |
| | Primary | primary | Supplementary |
| Endpoint | Estimand | estimand | Estimand 2 |

Co-primary

| | | | |
|---|---|---|---|
| Change from baseline | √ | √ | √ |
| through Week 4 in CSS (7- | | | |
| day average of total | | | |
| instantaneous AM scores for | | | |
| nasal congestion, facial pain | | | |
| or pressure, and nasal | | | |
| discharge (anterior and/or | | | |
| posterior)). | | | |
| Change from baseline to | √ | √ | √ |
| Week 24/ET in the APOV | | | |
| across the ethmoid and | | | |
| maxillary sinuses. | | | |

Key Secondary (Type 1 error controlled, pooled data)

| | | | |
|---|---|---|---|
| Change from baseline to | √ | √ | √ |
| Week 4/ET in CSS in | | | |
| subjects who were | | | |
| symptomatic despite use of | | | |
| a standard nasal steroid | | | |
| spray (e.g. Flonase, | | | |
| Nasonex) for treatment of | | | |
| CS at study entry | | | |
| Frequency of acute | √ | — | — |
| exacerbations of CS over | | | |
| the 24-week treatment | | | |
| period (defined as a | | | |
| worsening of symptoms | | | |
| requiring escalation of | | | |
| treatment) | | | |

Other Secondary

| | | | |
|---|---|---|---|
| Change from baseline in | √ | √ | √ |
| CSS over time (end of | | | |
| Weeks 8 and 12) | | | |
| Change from baseline to | √ | — | √ |
| Weeks 4, 8, and 12 on the 4 | | | |
| individual cardinal symptoms | | | |
| of CS: facial pain or | | | |
| pressure, nasal discharge | | | |
| (anterior and/or posterior), | | | |
| nasal congestion, and sense | | | |
| of smell | | | |
| Change from baseline to | √ | √ | — |
| Weeks 4, 8, 12, and 24/ET | | | |
| in disease-specific quality of | | | |
| life as measured by the | | | |
| SNOT-22 total score and | | | |
| subdomain scores (analyzed | | | |
| in each pivotal study) | | | |
| Change from baseline to | √ | √ | — |
| Week 24/ET in SNOT-22 | | | |
| total score in subjects who | | | |
| were symptomatic despite | | | |
| use of a standard nasal | | | |
| steroid spray (e.g., Flonase, | | | |
| Nasonex) for treatment of | | | |
| CS at study entry | | | |
| Time to first acute | √ | — | — |
| exacerbation of chronic | | | |
| sinusitis, defined as a | | | |
| worsening of symptoms that | | | |
| requires escalation of | | | |
| treatment | | | |
| Change from baseline to | √ | — | — |
| Week 24/ET in PSQI global | | | |
| and component scores (also | | | |
| at Week 12) | | | |
| Percent of subjects reporting | √ | — | — |
| disease improvement, as | | | |
| measured by PGIC at Week | | | |
| 4 and Week 24/ET | | | |

Abbreviations:
AM = morning;
APOV = average of the percentages of opacified volume;
CS = chronic sinusitis;
CSS = composite symptom score;
CT = computed tomography;
ET = early termination;
PGIC = Patient Global Impression of Change;
PSQI = Pittsburg Sleep Quality Index;
SNOT-22 = Sinonasal Outcome Test 22-item version

Hierarchical Closed Testing Procedure for Controlling Type I Error when Testing Co-Primary and Key Secondary Efficacy Endpoints

Testing was conducted to determine if a beneficial effect would be provided from administration of fluticasone propionate with the delivery device herein described relative to placebo. This assessment encompassed both the co-primary and key secondary endpoints; therefore, tests for these endpoints were included in the strategy for strong control of Type I error. The statistical analysis plan (SAP) for Study A (finalized prior to unblinding of either study) lists the hierarchy for the co-primary efficacy measures for that study and for two key secondary analyses to be performed in data pooled from both studies. The SAP for Study B applies only to the analyses related to that specific study; it was finalized prior to unblinding of Study B but after unblinding of Study A. Subsequent to this revision, in order to maintain strong Type 1 error control, an erratum to correct the final SAP for Study B was made to delete pooled analyses (i.e., analyses that included data from both studies) from the stepdown procedure because part of the data (from Study A) had already been unblinded at the time the Study B SAP was finalized. Therefore, only analyses specific to Study B data (and not pooled analyses) are included in the Study B stepdown procedure, and only the pooled analyses pre-specified in the Study A SAP are considered controlled for Type 1 error. The familywise Type 1 error probability for the test of the primary hypothesis was α=0.05. All statistical tests were two-sided tests at the 5% level of significance. Statistical significance was defined as P<0.05 (two-sided test). Nominal P-values were provided for all other efficacy endpoints not included in the formal testing strategy as well as efficacy endpoints within the formal testing strategy after a comparison failed to show a difference between the comparator and the active subgroup.

Analyses of Other Secondary Efficacy Analyses Endpoints

Changes from baseline to pre-specified time points for AM/PM instantaneous/reflective CSS and individual symptom scores, SNOT-22 total and domain scores, and PSQI global and component scores were analyzed using the same MMRM model described for the CSS co-primary endpoint analysis but with definitions of poor scores and baseline covariate changed appropriately.

For PGIC, the percentages of subjects “Improved” and the percentage “≥Much Improved” were compared between treatments (each active versus placebo) based on a logistic regression MMRM using the generalized linear model (GLM). The worst score of “Not Improved” was assigned for missing data for the composite strategy.

Assessment of Meaningful Within-Subject Change in APOV: An anchor-based method was used to estimate what constituted a clinically meaningful within-subject change from baseline to Week 24/ET in APOV. This analysis was done for the integrated dataset. The anchor for this analysis was a global patient-reported endpoint: PGIC at Week 24/ET. The mean change in APOV among patients reporting improvement was used as an estimated threshold for meaningful within-subject change in APOV. A subject was defined as a responder if the change from baseline to Week 24/ET was greater than or equal to the estimate of meaningful within-subject change. The percentage of subjects with a meaningful within-subject improvement in APOV at Week 24/ET was analyzed with a logistic regression model. For subjects with missing data at Week 24/ET, the change from baseline to Week 24/ET in APOV, which was based on an imputed value of APOV, was used to calculate the subject's response.

Subgroup Analyses

Subgroup analyses of the overall population by age (<65 years, 365 years), gender (male, female), race (White, Non-White), and region (North American, Non-North American) were performed on the co-primary endpoints (CSS, APOV) for the integrated dataset. The statistical models included the appropriate interaction terms for treatment-by-subgroup interaction as well as the appropriate contrasts to obtain the within-subgroup inferential statistics.

### Example 2: Study B

Subject Disposition, Demographics, and Baseline Characteristics

Informed consent was provided by 727 subjects; 475 subjects passed initial eligibility screening and entered the single-blind eligibility verification phase (including multi-day symptom diaries and CT scans); 223 of these subjects were subsequently randomized and 210 (94.2%) completed the study.

There was a similar distribution of age, race, and ethnicity among subjects in each treatment group. The mean age of subjects enrolled/randomized into the study was 48.4 years of age, with equal proportions of subjects enrolled by gender. The study population was predominantly White and not Hispanic or Latino (98.6% each). For the 3 treatment groups, mean body weights ranged from 75.7 to 82.1 kg, with the OPN-375 high dose group having the highest mean body weight.

At study entry, 25.7% of subjects were using a standard-delivery nasal steroid spray for treating their CS, with similar percentages across the 3 treatment groups (range: 21.9% to 28.0%). Previous sinus surgery (ethmoidectomy or maxillary antrostomy) was frequently reported in each of the 3 treatment groups, with prior sinus surgery in 36.0%, 34.2% and 35.1% of subjects in the placebo and low and high dose OPN-375 groups, respectively.

Nasal polyps were not present at study entry for subjects in this study (i.e., study subjects were phenotype CRSsNP). However, the overlapping continuum of chronic sinusitis disease between the visual phenotypes with and without polyps is reinforced and illustrated by the fact that a prior history of NP was reported for 19.4% of subjects in this study and ˜40% of subjects were reported to have “polypoid edema” on endoscopic examination. From an inflammatory endotype perspective, it is not possible to know the individual inflammatory profile of tissues inside the sinus cavities for subjects in this study; however, as previously noted, evidence now suggests that inflammatory profiles are heterogeneous and overlapping between CRSsNP and CRSwNP phenotypes. The most obvious conceptual difference between CRSsNP and CRSwNP phenotypes is obstruction and other symptoms that may be directly attributable to the physical presence of a polyp in the nasal cavity, and alteration of drug deposition by NP structures in the context of nasally delivered medication. Notably, because subjects in this study did not have nasal polyps at entry, both symptoms and changes in drug deposition that may be attributable to the physical presence of NP have been removed as a potential confounder of therapeutic effect.

Co-Primary Endpoints

Study B demonstrated superiority of active treatment with OPN-375 over placebo comparator on both of its co-primary endpoints (Tables 4 and 5).

Changes from Baseline to Week 4 in Composite Symptom Score

The differences between OPN-375 active treatment and placebo were statistically significant (P<0.05) for the symptom-based outcome of CSS (instantaneous AM) at Week 4 on the primary estimand (Table 4). Treatment differences were of similar magnitude and were statistically significant for analyses based on both the alternative primary estimand and SE-2 (Table 4). Analyses based on the other supplementary estimands and observed data (no imputation) also showed statistically significant treatment differences in favor of OPN-375. In the PPS, the treatment difference in the LS mean change from baseline (for each active dose minus placebo) remained statistically significant and in favor of both active groups. Robust (P<0.05) results in favor of both active treatment groups and extremely few missing observations for the primary estimand (2 missing observations at Week 4) precluded finding a tipping point; thus, the tipping point analyses were not performed.

TABLE 4
Change From Baseline to End of Week 4 in Composite Symptom Score
(Instantaneous AM).

| | | | Week 4 | | | | |
|---|---|---|---|---|---|---|---|
| | Baseline | | | LSM (SE) | | | |
| Treatment (BID) | n | Mean (SD) | n | CFB | LSMD | 95% CI | P-value |

Primary Estimand

| | | | | | | | |
|---|---|---|---|---|---|---|---|
| All OPN-375 | 145 | 5.92 | 143 | −1.64 | −0.83 | −1.32, −0.35 | <0.001 |
| | | (1.529) | | (0.146) | | | |
| OPN-375 372 μg | 73 | 5.97 | 71 | −1.74 | −0.93 | −1.49, −0.37 | 0.001 |
| | | (1.588) | | (0.204) | | | |
| OPN-375 186 μg | 72 | 5.87 | 72 | −1.54 | −0.73 | −1.29, −0.17 | 0.011 |
| | | (1.476) | | (0.204) | | | |
| Placebo | 75 | 6.15 | 72 | −0.81 | — | — | — |
| | | (1.768) | | (0.202) | | | |

Alternative primary estimand-Intercurrent Events Include Antibiotics for AECRS

| | | | | | | | |
|---|---|---|---|---|---|---|---|
| All OPN-375 | 145 | 5.92 | 143 | −1.61 | −0.88 | −1.37, −0.38 | <0.001 |
| | | (1.529) | | (0.149) | | | |
| OPN-375 372 μg | 73 | 5.97 | 71 | −1.69 | −0.95 | −1.53, −0.38 | 0.001 |
| | | (1.588) | | (0.208) | | | |
| OPN-375 186 μg | 72 | 5.87 | 72 | −1.53 | −0.80 | −1.37, −0.23 | 0.007 |
| | | (1.476) | | (0.209) | | | |
| Placebo | 75 | 6.15 | 72 | −0.73 | — | — | — |
| | | (1.768) | | (0.206) | | | |

Supplementary Estimand 2

| | | | | | | | |
|---|---|---|---|---|---|---|---|
| All OPN-375 | 145 | 5.92 | 143 | −1.64 | −0.81 | −1.30, −0.33 | 0.001 |
| | | (1.529) | | (0.146) | | | |
| OPN-375 372 μg | 73 | 5.97 | 71 | −1.74 | −0.91 | −1.47, −0.36 | 0.001 |
| | | (1.588) | | (0.204) | | | |
| OPN-375 186 μg | 72 | 5.87 | 72 | −1.54 | −0.71 | −1.27, −0.15 | 0.013 |
| | | (1.476) | | (0.204) | | | |
| Placebo | 75 | 6.15 | 73 | −0.82 | — | — | — |
| | | (1.768) | | (0.201) | | | |

Abbreviations:
AECRS = acute exacerbations of chronic rhinosinusitis;
AM = morning;
BID = twice daily;
CFB = change from baseline;
CI = confidence interval;
CSR = clinical study report;
LSM = least squares mean;
LSMD = least squares mean difference (active − placebo);
SD = standard deviation;
SE = standard error
Note:
Per the primary and supplementary estimands, 2 subjects in the placebo group and 1 subject in the high dose OPN-375 group had intercurrent events; for the alternative primary estimand 5 subjects in the placebo group and 2 subjects in the high dose OPN-375 group had intercurrent events.

Changes from Baseline to Week 24/ET in Average of the Percentages of Opacified Volume in the Ethmoid and Maxillary Sinuses (APOV)

For APOV, treatment differences were statistically significant for both active treatment doses compared with placebo on the primary estimand (Table 5). Tipping point analyses did not change the statistical significance of the treatment differences. Analyses based on the alternative primary estimand and SE-2 both showed treatment differences of similar magnitude which were also statistically significant in favor of both active treatment doses (Table 5). Treatment differences also remained statistically significant and in favor of both active groups for the other supplementary estimands (supplementary estimand 1 [SE-1], supplementary estimand 3 [SE-3], and supplementary estimand 4 [SE-4]), when a non-parametric ranked value analysis was done, when only observed data were considered, and using the PPS.

TABLE 5
Change from Baseline to Week 24/ET in Average of the Percentages
of Opacified Volume in Ethmoid and Maxillary Sinuses.

| | Baseline | | Week 24/ET | | | | |
|---|---|---|---|---|---|---|---|
| Treatment | | Mean | | LSM (SE) | | | |
| (BID) | n | (SD) | n | CFB | LSMD | 95% CI | P-value |

Primary Estimand

| | | | | | | | |
|---|---|---|---|---|---|---|---|
| All OPN- | 145 | 61.01 | 139 | −6.07 | −7.26 | −11.36, −3.16 | <0.001 |
| 375 | | (18.857) | | (1.236) | | | |
| OPN-375 | 73 | 61.50 | 69 | −5.14 | −6.33 | −11.08, −1.58 | 0.009 |
| 372 μg | | (18.460) | | (1.742) | | | |
| OPN-375 | 72 | 60.51 | 70 | −7.00 | −8.19 | −12.93, −3.45 | <0.001 |
| 186 μg | | (19.367) | | (1.725) | | | |
| Placebo | 75 | 64.09 | 66 | 1.19 | — | — | — |
| | | (17.741) | | (1.740) | | | |

Alternative primary estimand-Intercurrent Events Include Antibiotics for AECRS

| | | | | | | | |
|---|---|---|---|---|---|---|---|
| All OPN- | 145 | 61.01 | 139 | −4.20 | −7.52 | −11.82, −3.22 | <0.001 |
| 375 | | (18.857) | | (1.290) | | | |
| OPN-375 | 73 | 61.50 | 69 | −2.50 | −5.82 | −10.81, −0.83 | 0.022 |
| 372 μg | | (18.460) | | (1.801) | | | |
| OPN-375 | 72 | 60.51 | 70 | −5.89 | −9.22 | −14.15, −4.29 | <0.001 |
| 186 μg | | (19.367) | | (1.804) | | | |

Primary Estimand

| | | | | | | | |
|---|---|---|---|---|---|---|---|
| Placebo | 75 | 64.09 | 67 | 3.33 | — | — | — |
| | | (17.741) | | (1.829) | | | |

Supplementary Estimand 2

| | | | | | | | |
|---|---|---|---|---|---|---|---|
| All OPN- | 145 | 61.01 | 140 | −6.26 | −7.15 | −11.17, −3.12 | <0.001 |
| 375 | | (18.857) | | (1.223) | | | |
| OPN-375 | 73 | 61.50 | 70 | −5.48 | −6.36 | −11.01, −1.72 | 0.007 |
| 372 μg | | (18.460) | | (1.703) | | | |
| OPN-375 | 72 | 60.51 | 70 | −7.04 | −7.93 | −12.59, −3.27 | <0.001 |
| 186 μg | | (19.367) | | (1.710) | | | |
| Placebo | 75 | 64.09 | 67 | 0.88 | — | — | — |
| | | (17.741) | | (1.704) | | | |

Abbreviations:
AECRS = acute exacerbations of chronic rhinosinusitis;
BID = twice daily;
CFB = change from baseline;
CI = confidence interval;
CSR = clinical study report;
ET = early termination;
LSM = least squares mean;
LSMD = least squares mean difference (active − placebo);
SD = standard deviation;
SE = standard error
Note:
Per the primary and supplementary estimands, 12 subjects in the placebo group and 3 and 5 subjects in the low and high dose OPN-375 groups, respectively, had intercurrent events. For the alternative primary estimand, 17 subjects in the placebo group and 4 and 10 subjects in in the low and high dose OPN-375 groups, respectively, had intercurrent events.

Key Secondary Endpoint Analyses

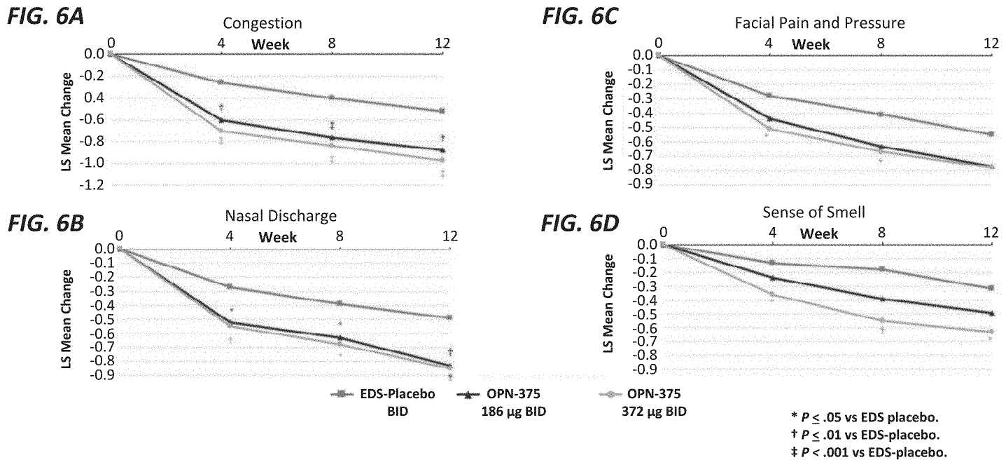

Change from baseline to the end of Week 4 in each of the 4 individual cardinal symptoms of CS (congestion/obstruction, facial pain or pressure, nasal discharge [anterior and/or posterior], and sense of smell) are summarized in Table 6 and FIGS. 6A-6D. For both active treatment groups, symptoms improved from baseline to the end of Week 4, and the magnitudes of the improvement were larger than observed for subjects treated with placebo. Between-group differences (active minus placebo) were larger for the high dose group than the low dose group for all symptoms and statistically significant for the high dose group for all 4 cardinal symptoms. The low dose group produced more improvement than the placebo comparator for all symptoms, reaching nominal statistical significance for 2 (congestion/obstruction and nasal discharge) of the 4 symptoms.

TABLE 6
Individual Cardinal Symptoms of Chronic Sinusitis (Change from Baseline to
End of Week 4).

| | | | Week 4 | | | | |
|---|---|---|---|---|---|---|---|
| | Baseline | | | LSM (SE) | | | |
| Treatment (BID) | n | Mean | n | CFB | LSMD | 95% CI | P-value |

Congestion/Obstruction

| | | | | | | | |
|---|---|---|---|---|---|---|---|
| All OPN-375 | 145 | 2.16 | 143 | −0.65 (0.055) | −0.40 | −0.58, −0.21 | <0.001 |
| OPN-375 372 μg | 73 | 2.21 | 71 | −0.70 (0.077) | −0.45 | −0.66, −0.24 | <0.001 |
| OPN-375 186 μg | 72 | 2.11 | 72 | −0.60 (0.077) | −0.34 | −0.56, −0.13 | 0.002 |
| Placebo | 75 | 2.26 | 72 | −0.26 (0.76) | — | — | — |

Nasal Discharge

| | | | | | | | |
|---|---|---|---|---|---|---|---|
| All OPN-375 | 145 | 2.04 | 143 | −0.53 (0.055) | −0.26 | −0.44, −0.08 | 0.005 |
| OPN-375 372 μg | 73 | 2.01 | 71 | −0.55 (0.077) | −0.28 | −0.49, −0.07 | 0.009 |
| OPN-375 186 μg | 72 | 2.06 | 72 | −0.52 (0.076) | −0.24 | −0.45, −0.04 | 0.022 |
| Placebo | 75 | 2.06 | 72 | −0.27 (0.76) | — | — | — |

Facial Pain/Pressure

| | | | | | | | |
|---|---|---|---|---|---|---|---|
| All OPN-375 | 145 | 1.72 | 143 | −0.47 (0.058) | −0.19 | −0.39, −0.00 | 0.048 |
| OPN-375 372 μg | 73 | 1.74 | 71 | −0.51 (0.081) | −0.23 | −0.45, −0.01 | 0.044 |
| OPN-375 186 μg | 72 | 1.70 | 72 | −0.44 (0.081) | −0.16 | −0.38, 0.06 | 0.160 |
| Placebo | 75 | 1.82 | 72 | −0.28 (0.080) | — | — | — |

Sense of Smell

| | | | | | | | |
|---|---|---|---|---|---|---|---|
| All OPN-375 | 145 | 2.07 | 143 | −0.30 (0.048) | −0.17 | −0.33, −0.01 | 0.033 |
| OPN-375 372 μg | 73 | 2.16 | 71 | −0.36 (0.067) | −0.23 | −0.41, −0.05 | 0.014 |
| OPN-375 186 μg | 72 | 1.98 | 72 | −0.24 (0.067) | −0.11 | −0.30, 0.07 | 0.219 |
| Placebo | 75 | 2.24 | 72 | −0.13 (0.066) | — | — | — |

Abbreviations:
BID = twice daily;
CI = confidence interval;
LSM = least squares mean;
LSMD = least squares mean difference (active − placebo);
SE = standard error

Subsequent to Week 4, during the treatment period in which symptomatic rescue medication was allowed, individual symptom scores generally continued to monotonically improve, with a sustained or growing magnitude of benefit of active treatment over the placebo comparator through the measurement period (Table 6).

The reduced incidence of AECRS may, in part, potentially result from the COVID-19 pandemic. Reductions in incidence of many acute upper respiratory infections were observed with obvious contributing factors including extended periods of “lockdowns”, mandatory masking in public, reduced seeking of healthcare for reasons unrelated to COVID-19, and other factors.

A comparison of active and placebo incidence rates of acute exacerbation was a separate pre-planned and Type-1 error-controlled analysis of pooled data from Studies A and B that was included as part of the step-down procedure for Study A; however, a separate analysis of the time-to-first-event that included only data from Study B was part of the closed testing procedure for control of Type 1 error in Study B. The length of time to the first AECRS, a key secondary endpoint in Study B, was defined as a worsening of symptoms that requires escalation of treatment. The restricted mean event time in the 186 μg OPN-375 group with 1 event was 5.00 weeks compared with 8.04 weeks for the 372 μg OPN-375 group which had 7 events and 9.83 weeks for the placebo group with 8 events. The log rank chi square p-value was 0.019 for the low dose group (nominal statistical significance), 0.621 for the high dose group (not statistically significant and breaking the closed testing procedure for Study B), and 0.104 for the combined OPN-375 group.

In Study B, the incidence rate ratio (IRR) of AECRS for all patients receiving OPN-375 versus patients receiving the placebo comparator was 0.28 (nominal P=0.036), reflecting an overall reduction in exacerbations of approximately 72%. In total, there were 7 subjects with 8 acute exacerbations of chronic sinusitis among 145 subjects in the FAS who received active treatment with OPN-375 (low dose group: 1 [1.4%] subject with 2 exacerbations and high dose group: 6 [8.2%] subjects) compared with 8 [10.7%] subjects with 13 acute exacerbations among 75 subjects in the FAS who received placebo.

Selected Secondary Endpoint Analysis

For the FAS overall population, the composite symptom score (AM instantaneous) continued to decrease/improve compared with baseline for all 3 treatment groups and the treatment difference in the LS mean change from baseline (for each active dose minus placebo) was nominally statistically significant and in favor of both doses at Week 4 (primary endpoint) and also in subsequent weeks after symptomatic rescue medication was allowed, specifically Week 8 and Week 12, in both dose groups (Table 7). The scores for all individual cardinal symptoms of chronic sinusitis (AM instantaneous) generally decreased/improved numerically compared to baseline in all 3 treatment groups in a similar fashion to the CSS, suggesting that the benefits of OPN-375 are not attributable to effect on only one symptom and that OPN-375 is affecting the underlying disease pathology in a way that provides benefit on each of the resulting core symptoms that define chronic sinusitis.

TABLE 7
Change from Baseline to Weeks 4, 8, and 12 in the 7-Day Average of
Instantaneous AM CSS.

| Week 4 | | Week 8 | | Week 12 | |
|---|---|---|---|---|---|
| OPN-375 Dose | | | | | |
| 186 μg | 372 μg | 186 μg | 372 μg | 186 μg | 372 μg |

Primary Estimand

| | | | | | | |
|---|---|---|---|---|---|---|
| LSM CFB | −1.54 | −1.74 | −2.02 | −2.19 | −2.47 | −2.60 |
| LSMD | −0.73 | −0.93 | −0.82 | −0.98 | −0.92 | −1.05 |
| 95% CI | −1.29, −0.17 | −1.49, −0.37 | −1.41, −0.24 | −1.57, −0.40 | −1.54, −0.31 | −1.66, −0.44 |
| P-value | 0.011 | 0.001 | 0.006 | 0.001 | 0.003 | <0.001 |

Alternative primary estimand-Intercurrent Events Including Antibiotics for AECRS

| | | | | | | |
|---|---|---|---|---|---|---|
| LSM CFB | −1.53 | −1.69 | −2.00 | −1.84 | −2.45 | −2.12 |
| LSMD | −0.80 | −0.95 | −1.04 | −0.88 | −1.28 | −0.95 |
| 95% CI | −1.37, −0.23 | −1.53, −0.38 | −1.70, −0.38 | −1.53, −0.22 | −1.99, −0.56 | −1.66, −0.23 |
| P-value | 0.007 | 0.001 | 0.002 | 0.009 | <0.001 | 0.010 |

Abbreviations:
AECRS = acute exacerbations for chronic rhinosinusitis;
AM = morning;
CFB = change from baseline;
CI = confidence interval;
CSS = composite symptom score;
LSM = least squares mean;
LSMD = least squares mean difference

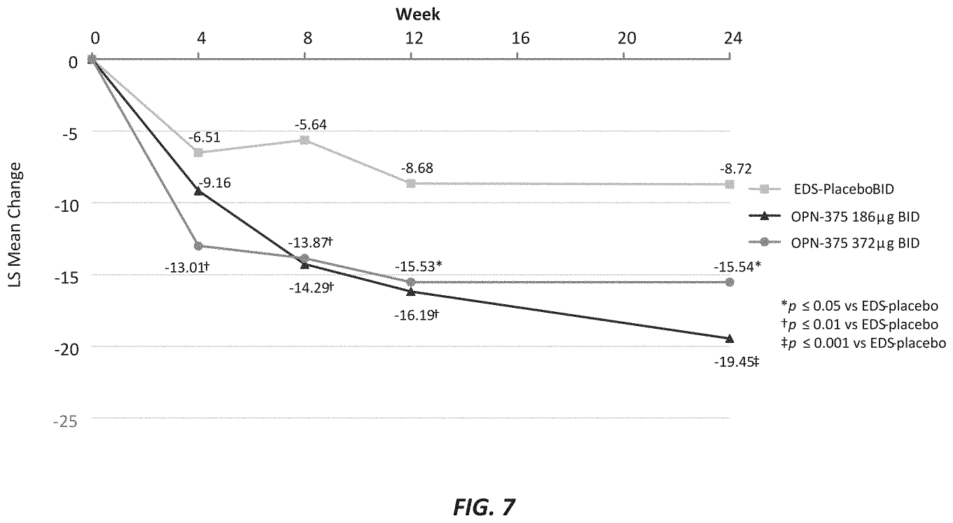

Results of the disease-specific quality of life instrument SNOT-22 corroborated the results observed using the CSS and individual symptom scores, showing improvement in symptoms and disease-specific quality of life with active treatment with OPN-375 (FIG. 7 and Table 8). For the SNOT-22 total score, the LS mean changes from baseline to Weeks 4, 8, 12, and 24/ET for both OPN-375 groups were greater than the MCID of 8.9 points. Nominal statistically significant treatment differences were achieved for both doses of OPN-375 at Weeks 8, 12 and 24/ET; and for high dose OPN-375 at Week 4.

TABLE 8
Change from Baseline in Sinonasal Outcome Test-22 Total Score.

| Week 4 | | Week 8 | | Week 12 | | Week 24 | |
|---|---|---|---|---|---|---|---|
| OPN-375 Dose μg BID | | | | | | | |
| 186 | 372 | 186 | 372 | 186 | 372 | 186 | 372 |

Primary Estimand

| | | | | | | | | |
|---|---|---|---|---|---|---|---|---|
| LSM CFB | −9.16 | −13.01 | −14.29 | −13.87 | −16.19 | −15.53 | −19.45 | −15.54 |
| LSMD | −2.65 | −6.50 | −8.65 | −8.24 | −7.51 | −6.85 | −10.72 | −6.81 |
| 95% CI | −7.43, | −11.29, | −14.10, | −13.71, | −13.10, | −12.46, | −16.79, | −12.92, |
| | 2.13 | −1.71 | −3.20 | −2.77 | −1.93 | −1.24 | −4.65 | −0.71 |
| P-value | 0.276 | 0.008 | 0.002 | 0.003 | 0.009 | 0.017 | <0.001 | 0.029 |

Alternative primary estimand-Intercurrent Events Include Antibiotics for AECRS

| | | | | | | | | |
|---|---|---|---|---|---|---|---|---|
| LSM CFB | −8.94 | −12.33 | −13.56 | −12.42 | −15.73 | −13.57 | −18.19 | −13.44 |
| LSMD | −3.60 | −6.98 | −9.01 | −7.97 | −9.02 | −6.85 | −11.64 | −6.89 |
| 95% CI | −8.53, | −11.92, | −14.79, | −13.68, | −14.95, | −12.81, | −18.06, | −13.34, |
| | 1.33 | −2.03 | −3.41 | −2.25 | −3.08 | −0.89 | −5.23 | −0.44 |
| P-value | 0.152 | 0.006 | 0.002 | 0.007 | 0.003 | 0.025 | <0.001 | 0.036 |

Abbreviations:
AECRS = acute exacerbations for chronic rhinosinusitis;
BID = twice daily;
CFB = change from baseline;
CI = confidence interval;
LSM = least squares mean;
LSMD = least squares mean difference.

Individual domain scores of the SNOT-22 also generally reflected clinical improvement with active treatment. LS mean changes from baseline for both OPN-375 groups were numerically larger in magnitude compared with placebo at most timepoints. With few exceptions, the change from baseline treatment differences (active dose minus placebo) for SNOT-22 rhinologic symptoms, extra-nasal rhinologic symptoms, and ear/facial symptoms domain scores were nominally statistically significant (P<0.05) in favor of active treatment at timepoints starting at Week 4 up through the end of the double-blind treatment phase. For the psychological dysfunction domain scores, the treatment differences were of nominal statistical significance for the low dose group at most timepoints, except for Week 4; the treatment differences for the high dose group did not meet nominal statistical significance. For the sleep dysfunction domain scores, treatment differences (active dose minus placebo) at Weeks 8, 12, and 24/ET were of nominal statistical significance in favor of 186 μg OPN-375 (P<0.05) and at Weeks 8 and 24/ET for 372 μg OPN-375.

Mean Pittsburgh Sleep Quality Index (PSQI) Global Scores decreased (i.e., improved) from baseline to both Week 12 and Week 24/ET in all 3 treatment groups, and the treatment differences (active group minus placebo) reached nominal statistical significance (P<0.05) in favor of both active treatment groups at Week 12 and for the 186 μg OPN-375 group, alone, at Week 24/ET.

Treatment differences (active group minus placebo) were nominally statistically significant in favor of the 186 μg OPN-375 group at Week 24/ET for most PSQI component scores (subjective sleep quality, sleep latency, sleep duration, sleep efficiency, sleep disturbance, and daytime dysfunction). For daytime dysfunction, the treatment difference was nominally statistically significant in favor of both active groups at Week 24/ET and, for subjective sleep quality, these differences were nominally statistically significant in favor of both active groups at Week 12 (and for the 186 μg OPN-375 group at Week 24/ET).

A summary of the number (%) of subjects indicating improvement (very much improved or much improved or minimally improved) versus not improved (no change, any degree of worsening, or missing data) is summarized in Table 9. For both active treatment groups, the odds ratios for patients reporting that they feel improved (vs the placebo group) were nominally statistically significant at Week 24/ET at both active doses (P=0.004 low dose; P<0.001 high dose). Using a narrower definition of improved (very much improved or much improved), the odds ratios for improvement in the active groups versus placebo were nominally statistically significant (P<0.001) for both active groups at both Week 4 and Week 24. These findings indicate that, from a global patient-reported perspective, there was a clear patient perception of improvement in CS disease for subjects receiving OPN-375 compared to subjects receiving placebo.

TABLE 9
Change from Baseline in Patient Global Impression of Change Over Time.

| | | | OPN-375 | OPN-375 |
|---|---|---|---|---|
| | | Placebo | 186 μg | 372 μg |
| Time Point | Category | (N = 75) | (N = 72) | (N = 73) |
| Week 4 | n | 72 | 71 | 71 |
| | Improved | 37 (51.4) | 47 (66.2) | 56 (78.9) |
| | Very much improved | 0 | 6 (8.5) | 4 (5.6) |
| | Much improved | 8 (11.1) | 22 (31.0) | 24 (33.8) |
| | Minimally improved | 29 (40.3) | 19 (26.8) | 28 (39.4) |
| | Not Improved | 35 (48.6) | 24 (33.8) | 15 (21.1) |
| | Odds Ratio (Improved) | — | 1.85 | 3.52 |
| | P-value | — | 0.078 | 0.001 |
| Week 24/ET | n | 69 | 70 | 69 |
| | Improved | 42 (60.9) | 59 (84.3) | 63 (91.3) |
| | Very much improved | 3 (4.3) | 19 (27.1) | 12 (17.4) |
| | Much improved | 14 (20.3) | 25 (35.7) | 27 (39.1) |
| | Minimally improved | 25 (36.2) | 15 (21.4) | 24 (34.8) |
| | Not Improved | 27 (39.1) | 11 (15.7) | 6 (8.7) |
| | Odds Ratio (Improved) | — | 3.36 | 6.81 |
| | P-value | — | 0.004 | <0.001 |

Abbreviations:
ET = early termination;
MMRM = mixed model for repeated measures
Note:
The denominator for each visit is the number of subjects with an assessent at the visit. Inferential statistics are based on a logistic MMRM including categorical effects for previous sinus surgery, nasal polyp status, treatment, week (4, 24/ET), and treatment-by-week interaction.

Adverse Events

Adverse events (AEs) were reported for 45.6% of OPN-375 recipients (32.9% of subjects in the 186 μg group and 58.1% of subjects in the 372 μg group) and 41.3% of placebo recipients. In OPN-375 recipients, events with a MedDRA PT of “epistaxis” were the most frequently reported treatment-emergent adverse events (TEAEs) during the double-blind treatment phase (7.5% of combined OPN-375-treated subjects). Overall, epistaxis was reported as a TEAE by 4 (5.5%), 7 (9.5%), and 0 (0%) subjects in the 186 μg OPN-375, 372 μg OPN-375, and placebo groups, respectively. Epistaxis was also the only TEAE reported at >5% difference in the all OPN-375 group compared with the placebo group. All 4 cases of epistaxis in the 186 ug OPN-375 group were considered mild in severity. The AEs of epistaxis reported for 7 subjects in the 372 ug OPN-375 group were considered mild in 6 subjects and moderate in severity for 1 subject. For TEAEs considered treatment related by the investigator, epistaxis was the most commonly reported related event in the active groups; it was reported in 4 (5.5%) and 6 (8.1%) subjects in the 186 μg OPN-375 and 372 μg OPN-375 groups, respectively, compared with 0 subjects in the placebo group. Four subjects reported a TEAE leading to randomized study treatment discontinuation: 1 (1.4%), 1 (1.4%), and 2 (2.7%) subjects in the 186 μg OPN-375, 372 μg OPN-375, and placebo groups, respectively. There was no discernible pattern in the TEAEs leading to discontinuation.

Severe AEs were reported in 4 subjects (1 subject in the 186 μg group, 2 subjects in the 372 μg group, and 1 placebo recipients). No severe event was considered related to study treatment. Five subjects had a total of 5 SAEs (1 subject in the 186 μg OPN-375 group and 4 subjects in the 372 μg OPN-375 group). No SAE was fatal, and none was considered related to study treatment.

A total of 12 subjects (0 in the placebo group, 3 in the 186 μg OPN-375 group, and 9 in the 372 μg OPN 375 group) had a posttreatment finding of bleeding. Three of these 12 subjects (1 in the 186 μg OPN-375 group and 2 in the 372 μg OPN-375 group) had active bleeding (including a single subject in the 372 μg OPN-375 group who had findings of both non-active and active bleeding), and 9 had only non-active bleeding. Epistaxis was reported as a TEAE for 2 of the 3 subjects with findings of active bleeding; the majority of these TEAEs were mild, and none were severe. No subjects discontinued study drug due to epistaxis, and none of the subjects with non-active bleeding required medical intervention or treatment.

Two subjects in the OPN 375 groups had evidence of septal erosion (1 subject in the 186 μg group, and 1 subject in the 372 μg group), and 1 subject in the placebo group had a septal ulceration found on Week 24/ET nasal exam. Ulceration/erosion on areas other than septum was observed in 1 subject in the placebo group at Week 12. The finding was an abnormally eroded epithelial surface at the head of middle turbinate and was not considered clinically significant because it was expected to resolve quickly. Mucosal candidiasis was not present in any subject at any time during the study. The incidence of new cataracts overall was not different between subjects receiving OPN-375 and placebo. There were no clinically relevant differences in vital signs (BP and pulse) observed in this study between the OPN-375 treatment groups and the placebo group. There were no obvious differences in concomitant medication use across treatment groups.

Discussion

Treatment with OPN-375 (both 186 and 372 μg BID) produced significant improvements for a population with CT-scan verified CS who did not have polyps in the nasal cavity. Statistically and clinically significant improvement compared to a placebo comparator was demonstrated in pre-specified co-primary subjective (symptom relief at Week 4) and objective (decreased opacity in sinus cavities at Week 24/ET) endpoints. Treatment differences on these primary measures of efficacy were robust to analytic approach.

The demonstration of robust statistically significant benefits on both CSS and APOV provides strong evidence that OPN-375 improves the symptoms, and intra-sinus disease as measured by sinus opacification, for this population of patients with CS without NP. The clinical meaningfulness of the benefit is also supported by a variety of secondary outcomes measured in this trial that characterized the improvement more broadly, particularly from the patient's perspective, including each individual cardinal symptom of chronic sinusitis, disease-specific quality of life (SNOT-22), and a global measure of patient-reported change in disease (PGIC).

In this trial, CT scans assessing sinus opacification served as a biomarker for the effect of treatment at a specific site of action—the sinus cavities. The fact that statistically significant improvement in sinus opacification was observed in this trial substantiates the conclusion that intra-sinus inflammation is a site of action with drug treatment with OPN-375, and more specifically that treatment effects of OPN-375 are not limited to the nasal cavity as might be expected with an intranasally administered drug product that is known to be effective for a nasal cavity indication (nasal polyps).

Combined symptom score change over time demonstrates treatment improvement was sustained through Week 12 (the last measurement timepoint for CSS and individual symptom scales). Each of the 4 individual cardinal symptoms of chronic sinusitis also improved starting at Week 4, with the mean change for each of the individual symptoms following a similar pattern over time, indicating that benefits are not primarily the consequence of treating a single symptom. The CSS and individual symptom findings from AM reflective, PM instantaneous, and PM reflective symptom assessments were all consistent with the primary AM instantaneous findings, supporting the primary finding and demonstrating that the treatment benefits of OPN-375 are consistent throughout the day and supporting a BID dosing regimen.

AECRS are a major source of disability for the roughly 30 million patients in the US who suffer from chronic sinusitis, and reduction of acute exacerbations is one of the core domains that patients define as part of their perception of disease control. Exacerbations not only contribute directly to poor quality of life and lack of patient-defined disease control, but also push people to frequent use of antibiotics, oral steroids, or even surgery. In this study, all but one acute exacerbation was associated with use of antibiotics, and there was a large magnitude of reduction (72%) in AECRS among patients receiving OPN-375 compared to those receiving the placebo comparator. Data suggests that treatment of CRS is the top reason for adult outpatient antibiotic prescribing in the US, and reduction in antibiotic use enhances antibiotic stewardship, reduces risks for adverse drug reactions and changes to nasal or GI microbiota, and reduces risk for emergence of antibiotic resistant organisms. Particularly in the context where no other medications are known to reduce AECRS, and no drug treatments have been approved with this benefit, this reduction in acute exacerbations of CS is further evidence of a clinically important benefit of OPN-375 for individuals with CS and, from a public health perspective, for society more broadly.

Treatment with OPN-375 was well tolerated. The incidence of TEAEs observed in this trial was also similar to that reported with other intranasally administered topical steroids when studied in similar populations for similar durations.

Together, the positive results on the primary endpoint of combined symptoms, on multiple additional measures, including individual cardinal symptoms and other patient-reported benefits, such as disease-specific and general health-related quality of life, and on AECRS, all indicate that there are statistically significant and clinically meaningful benefits for patients with CS who use OPN-375. The fact that objective imaging (CT scan) as a biomarker for clinical effects occurring inside the sinus cavities (as distinct from the nasal cavity) also showed a statistically significant improvement with OPN-375 substantiates the conclusion that the clinical benefits can be attributed to treatment of sinus disease and not only to treatment of nasal disease. In addition, the exclusion from this study of chronic sinusitis subjects with concurrent observation of polyps in the nasal cavity is evidence that the treatment benefit cannot be attributed to the treatment of nasal polyps. These treatment benefits demonstrate a new and previously unlabeled benefit not attributable only to treatment of the nasal cavity or only to treatment of polyps inside the nasal cavity.

### Example 3: Study A

Subject Disposition, Demographics, and Baseline Characteristics

Informed consent was provided by 919 subjects of whom 332 met enrollment criteria (including symptoms and objective CT scan evidence of sinus disease), were randomized, and received at least 1 dose of study drug. A total of 124 (124/332, 37.3%) of the randomized chronic sinusitis subjects did not have observable polyps inside the nasal cavity at baseline (Table 10). Most subjects (˜90%) completed 24 weeks of double-blind study treatment. Of the subjects that discontinued study treatment, the most common reason given for discontinuation of study drug was lack of efficacy, although this occurred in <5% of subjects overall. The disposition for the overall population and for subjects without observable polyps inside the nasal cavity was similar.

TABLE 10
Disposition of All Enrolled Subjects and Those Without Nasal Polyps.

| | All Enrolled | Without Nasal Polyps |
|---|---|---|
| Analysis Group | Total | Total |
| Enrolled Subjects | 919 | 448 |
| Single-blind Run-in Set | 556 | 277 |
| ITT | 332 (100) | 124 (100) |
| Safety | 332 (100) | 124 (100) |
| Full Analysis Set | 327 (98.5) | 122 (98.4) |
| Per-protocol | 252 (75.9) | 90 (72.6) |
| Completed Study | 299 (90.1) | 115 (92.7) |
| Discontinued Study | 33 (9.9) | 9 (7.3) |
| AE | 7 (2.1) | 1 (0.8) |
| Lost to follow-up | 4 (1.2) | 0 |
| Lack of efficacy | 15 (4.5) | 4 (3.2) |
| Protocol violation | 1 (0.3) | 1 (0.8) |
| Withdrawal by subject | 2 (0.6) | 2 (1.6) |
| Other | 4 (1.2) | 1 (0.8) |

Abbreviations:
AE = adverse event;
ITT = intent-to-treat

There was a similar distribution of age, race, and ethnicity among subjects in each randomized treatment group. Overall, the mean age of randomized subjects was 49.1 years. Slightly more than half (57.5%) were male, and 42.5% were female. The population was predominantly White (90.1%). Demographic characteristics were similar for subjects without polyps observed in the nasal cavity, and similar across randomized treatment groups. The proportion of subjects who were reported to have NP at baseline and the proportion who had a history of previous sinus surgery, both of which were variables for which randomization was stratified, were comparable between the 3 treatment groups. A high percentage (48.5%) of subjects reported using at least 1 standard-delivery nasal steroid for CS at study entry.

Co-Primary Endpoint Analysis

Study A demonstrated superiority of active treatment of OPN-375 over the placebo comparator for both pre-specified co-primary endpoints (Tables 11 and 12).

Change from Baseline to Week 4 in Composite Symptom Score (Instantaneous AM)

The differences between OPN-375 and placebo were statistically significant (P<0.05) for the symptom-based outcome of CSS (instantaneous AM) at Week 4 using the primary estimand, alternative primary estimand, and SE-2, with all showing similar magnitude of benefit in favor of OPN-375 (Table 11). For the other supplementary estimands, SE-1, SE-3, and SE-4, observed data (no imputations) and the per-protocol population treatment differences were also statistically significant and in favor of both active groups (P<0.001 for both comparisons). For the primary estimand, with P<0.001 for both dose groups, there were minimal (only 5) missing observations. The lack of missing data precluded reaching a tipping point; therefore, tipping point analyses were not done.

TABLE 11
Change in Composite Symptom Score (Baseline to Week 4 [7-Day Average,
Instantaneous AM]).

| | | | Week 4 | | | | |
|---|---|---|---|---|---|---|---|
| Treatment | Baseline | | | LSM (SE) | | | |
| (BID) | n | Mean (SD) | n | CFB | LSMD | 95% CI | P-value |

Primary Estimand

| | | | | | | | |
|---|---|---|---|---|---|---|---|
| All OPN-375 | 217 | 5.45 (1.817) | 214 | −1.59 (0.116) | −0.97 | −1.36, −0.59 | <0.001 |
| OPN-375 372 μg | 107 | 5.48 (1.833) | 106 | −1.60 (0.163) | −0.98 | −1.43, −0.54 | <0.001 |
| OPN-375 186 μg | 110 | 5.42 (1.809) | 108 | −1.58 (0.161) | −0.97 | −1.41, −0.52 | <0.001 |
| Placebo | 110 | 5.77 (1.775) | 108 | −0.62 (0.161) | — | — | — |

Alternative primary estimand-Intercurrent Events Include Antibiotics for AECRS

| | | | | | | | |
|---|---|---|---|---|---|---|---|
| All OPN-375 | 217 | 5.45 (1.817) | 214 | −1.55 (0.126) | −1.23 | −1.65, −0.81 | <0.001 |
| OPN-375 372 μg | 107 | 5.48 (1.833) | 106 | −1.58 (0.178) | −1.25 | −1.74, −0.77 | <0.001 |
| OPN-375 186 μg | 110 | 5.42 (1.809) | 108 | −1.52 (0.176) | −1.20 | −1.68, −0.72 | <0.001 |
| Placebo | 110 | 5.77 (1.775) | 108 | −0.32 (0.176) | — | — | — |

Supplementary Estimand 2

| | | | | | | | |
|---|---|---|---|---|---|---|---|
| All OPN-375 | 217 | 5.45 (1.817) | 215 | −1.63 (0.110) | −0.95 | −1.32, −0.59 | <0.001 |
| OPN-375 372 μg | 107 | 5.48 (1.833) | 106 | −1.65 (0.154) | −0.97 | −1.39, −0.55 | <0.001 |
| OPN-375 186 μg | 110 | 5.42 (1.809) | 109 | −1.61 (0.152) | −0.94 | −1.36, −0.52 | <0.001 |
| Placebo | 110 | 5.77 (1.775) | 108 | −0.68 (0.153) | — | — | — |

Abbreviations:
AECRS = acute exacerbations of chronic rhinosinusitis;
AM = morning;
BID = twice daily;
CFB = change from baseline;
CI = confidence interval;
CSR = clinical study report;
LSM = least squares mean;
LSMD = least squares mean difference (active − placebo);
SD = standard deviation;
SE = standard error
Note:
Per the primary estimand and supplementary estimand 2, 5 subjects in the placebo group and 3 and 5 subjects in the low and high dose OPN-375 groups, respectively, had intercurrent events. For the alternative primary estimand, 14 subjects in the placebo group and 4 and 6 subjects in in the low and high dose OPN-375 groups, respectively, had intercurrent events.

Change from Baseline to Week 24/ET in Average of the Percentages of Opacification in the Ethmoid and Maxillary Sinuses (APOV)

For APOV, treatment differences were statistically significant for both active treatment doses compared with placebo on the primary estimand, the alternative primary estimand, and SE-2, with a similar magnitude of benefit associated with active treatment (Table 12). For the other supplementary estimands, nominal statistically significant treatment differences were found for the combined dose of active treatment and either both dose groups or the high dose OPN-375 alone. Tipping point analyses found that the tipping points were 0.5 and 1.6 times the within-group standard deviation (SD) for the low and high dose groups, respectively. The difference between active and placebo treatment as measured by APOV was also statistically significant when analyzed using ranked values (non-parametrically) and with observed data (no imputation). In the PPS, the treatment differences remained statistically significant and in favor of the 372 μg OPN-375 group.

TABLE 12
Change in Average of Percentages of Opacified Volume in Ethmoid and
Maxillary Sinuses (Baseline to Week 24/ET).

| | | | Week 24/ET | | | | |
|---|---|---|---|---|---|---|---|
| Treatment | Baseline | | | LSM (SE) | | | |
| (BID) | n | Mean (SD) | n | CFB | LSMD | 95% CI | P-value |

Primary Estimand

| | | | | | | | |
|---|---|---|---|---|---|---|---|
| All OPN-375 | 217 | 68.91 (19.14) | 199 | −5.89 (1.03) | −4.28 | −7.61, −0.96 | 0.012 |
| OPN-375 372 μg | 107 | 68.95 (18.84) | 101 | −6.20 (1.41) | −4.59 | −8.41, −0.78 | 0.018 |
| OPN-375 186 μg | 110 | 68.88 (19.51) | 98 | −5.58 (1.44) | −3.98 | −7.86, −0.09 | 0.045 |
| Placebo | 110 | 68.94 (20.33) | 99 | −1.60 (1.42) | — | — | — |

Alternative primary estimand-Intercurrent Events Include Antibiotics for AECRS

| | | | | | | | |
|---|---|---|---|---|---|---|---|
| All OPN-375 | 217 | 68.91 (19.14) | 202 | −2.64 (1.10) | −5.24 | −8.78, −1.70 | 0.004 |
| OPN-375 372 μg | 107 | 68.95 (18.84) | 101 | −3.86 (1.52) | −6.45 | −10.53, −2.38 | 0.002 |
| OPN-375 186 μg | 110 | 68.88 (19.51) | 101 | −1.43 (1.53) | −4.03 | −8.15, 0.09 | 0.055 |
| Placebo | 110 | 68.94 (20.33) | 100 | 2.60 (1.50) | — | — | — |

Supplementary Estimand 2

| | | | | | | | |
|---|---|---|---|---|---|---|---|
| All OPN-375 | 217 | 68.91 (19.14) | 207 | −6.49 (1.03) | −4.23 | −7.62, −0.85 | 0.014 |
| OPN-375 372 μg | 107 | 68.95 (18.84) | 105 | −6.26 (1.44) | −4.00 | −7.89, −0.11 | 0.044 |
| OPN-375 186 μg | 110 | 68.88 (19.51) | 102 | −6.73 (1.45) | −4.47 | −8.42, −0.52 | 0.027 |
| Placebo | 110 | 68.94 (20.33) | 104 | −2.26 (1.44) | — | — | — |

Abbreviations:
AECRS = acute exacerbations of chronic rhinosinusitis;
AM = morning;
BID = twice daily;
CFB = change from baseline;
CI = confidence interval;
CSR = clinical study report;
ET = early termination;
LSM = least squares mean;
LSMD = least squares mean difference (active − placebo);
SD = standard deviation;
SE = standard error
Note:
Per the primary estimand and supplementary estimand 2, 21, 17 and 11 subjects in the placebo, low and high dose OPN-375 groups had intercurrent events. Per the alternative primary estimand, 34, 28, and 17 subjects in the placebo, low and high dose OPN-375 groups had intercurrent events.

Key Secondary Endpoints

As part of the closed testing procedure for strong control of Type 1 error in Study A, certain key secondary efficacy endpoints using pooled data from Studies A and B were analyzed immediately after the co-primary endpoints.

Acute exacerbations were defined as an episode in which the patient experienced worsening of one or more of the core symptoms of CS (congestion, discharge, facial pain/pressure, loss of sense of smell) for a minimum of 3 days that also resulted in escalation of care (e.g., prescribing of an antibiotic or systemic steroid, acute care visit, ER visit). Pre-planned and Type 1 error-controlled analysis of the frequency of acute sinus exacerbations over the 24-week treatment period compared OPN-375 dose groups and placebo in pooled data from Studies A and B and used a negative binomial regression (NB2) model. The NB2 model used family-wise Type 1 error control and included the logarithm of exposure time as an offset variable.

In total, there were 76 exacerbations, of which 71 resulted in the use of antibiotics and 13 resulted in the use of oral steroids. Treatment with OPN-375 produced a large magnitude and statistically significant reduction in the incidence of disease exacerbations (Table 13). Specifically, exacerbations for all patients receiving OPN-375 were reduced by 61% compared to exacerbations among patients receiving placebo, with statistically significant reductions of 56% and 66% for the low and high doses, respectively.

TABLE 13
Frequency of Acute CRS Exacerbations over 24 Weeks.

| | | | LSM (SE) | IRR | |
|---|---|---|---|---|---|
| Treatment | | | Events/Patient | (Active/Placebo) | |
| Group (BID) | n | Events | (24 wks) | (95% CI) | P-value |
| All OPN-375 | 362 | 35 | 0.081 (0.0177) | 0.389 (0.226, 0.669) | 0.001 |
| OPN-375 | 180 | 15 | 0.072 (0.0218) | 0.343 (0.173, 0.680) | 0.002 |
| 372 μg | | | | | |
| OPN-375 | 182 | 20 | 0.092 (0.0255) | 0.441 (0.233, 0.832) | 0.012 |
| 186 μg | | | | | |
| Placebo | 185 | 41 | 0.208 (0.0446) | — | — |

Abbreviations:
BID = twice daily;
CI = confidence interval;
CRS = chronic rhinosinusitis;
IRR = incidence rate ratio;
LSM = least squares mean;
SE = standard error;
wks = weeks
Note:
Inferential statistics are based on a negative binomial regression (NB2) model including categorical effects for protocol, nasal polyp status, previous sinus surgery, treatment, and natural logarithm of treatment duration (days) as an offset parameter.

In the subpopulation of CS patients who did not have polyps in the nasal cavity at baseline, treatment with OPN-375 produced a reduction in the rate of acute exacerbations of chronic rhinosinusitis (AECRS) compared to placebo of a roughly similar magnitude (˜53%; P=0.032) as observed in the overall population. There was a similar reduction in events in both dose groups (Table 14).

TABLE 14
Frequency of Acute CRS Exacerbations by Phenotype.

| | | | | | |
|---|---|---|---|---|---|
| Population | | | LSM (SE) | IRR | |
| Treatment | | | Events/Patient | (Active/Placebo) | |
| Group (BID) | n | Events | (24 wks) | (95% CI) | P-value |

CS Patients With Nasal Polyps

| | | | | | |
|---|---|---|---|---|---|
| All OPN-375 | 137 | 12 | 0.052 (0.0197) | 0.276 (0.112, 0.679) | 0.005 |
| OPN-375 | 68 | 4 | 0.038 (0.0215) | 0.203 (0.060, 0.685) | 0.010 |
| 372 μg | | | | | |
| OPN-375 | 69 | 8 | 0.070 (0.0311) | 0.376 (0.138, 1.022) | 0.055 |
| 186 μg | | | | | |
| Placebo | 69 | 17 | 0.187 (0.0673) | — | — |

CS Patients Without Nasal Polyps

| | | | | | |
|---|---|---|---|---|---|
| All OPN-375 | 225 | 23 | 0.113 (0.0270) | 0.472 (0.237, 0.939) | 0.032 |
| OPN-375 | 112 | 11 | 0.113 (0.0386) | 0.470 (0.204, 1.086) | 0.077 |
| 372 μg | | | | | |
| OPN-375 | 113 | 12 | 0.113 (0.0376) | 0.474 (0.208, 1.081) | 0.076 |
| 186 μg | | | | | |
| Placebo | 116 | 24 | 0.239 (0.0619) | — | — |

Abbreviations:
BID = twice daily;
CI = confidence interval;
CRS = chronic rhinosinusitis;
CS = chronic sinusitis;
IRR = incidence rate ratio;
LSM = least squares mean;
SE = standard error;
wks = weeks
Note:
Inferential statistics are based on a negative binomial regression (NB2) model including categorical effects for protocol, nasal polyp status, previous sinus surgery, treatment, and natural logarithm of treatment duration (days) as an offset parameter, with treatment-by-subgroup interaction.
Source: ISE Table 14.2.3.2

CSS change from baseline through Week 4 in the subgroup of subjects entering the trial already using a standard-delivery nasal steroid for treatment of CS was analyzed using the same MMRM model used for the coprimary endpoint of CSS.

Baseline scores were similar across treatment groups. Both doses of OPN-375 produced significant improvement compared to placebo in this population, a finding that was robust to analytic approach (primary, alternative primary, and SE-2 estimands), with a magnitude similar to the symptom improvement observed in the overall population, particularly using alternative primary estimand (Table 15). This suggests that there is a treatment effect of OPN-375 in CS patients entering these trials, independent of whether or not patients were symptomatic while using standard-delivery nasal steroids.

TABLE 15
Composite Symptom Scores for Patients Using Standard-Delivery Nasal
Steroid at Trial Entry (Change for Baseline to End of Week 4).

| | | | Week 4 | | | | |
|---|---|---|---|---|---|---|---|
| Treatment | Baseline | | | LSM (SE) | | | |
| (BID) | n | Mean (SD) | n | CFB | LSMD | 95% CI | P-value |

Primary Estimand

| | | | | | | | |
|---|---|---|---|---|---|---|---|
| All OPN-375 | 172 | 5.63 (1.744) | 169 | −1.46 (0.132) | −0.70 | −1.09, −0.30 | <0.001 |
| OPN-375 372 μg | 85 | 5.54 (1.749) | 83 | −1.40 (0.184) | −0.63 | −1.10, −0.16 | 0.009 |
| OPN-375 186 μg | 87 | 5.71 (1.746) | 86 | −1.53 (0.180) | −0.76 | −1.23, −0.30 | 0.001 |
| Placebo | 108 | 5.84 (1.730) | 104 | −0.77 (0.163) | — | — | — |

Alternative primary estimand-Intercurrent Events Include Antibiotics for AECRS

| | | | | | | | |
|---|---|---|---|---|---|---|---|
| All OPN-375 | 172 | 5.63 (1.744) | 169 | −1.47 (0.144) | −1.03 | −1.46, −0.60 | <0.001 |
| OPN-375 372 μg | 85 | 5.54 (1.749) | 83 | −1.39 (0.201) | −0.95 | −1.46, −0.44 | <0.001 |
| OPN-375 186 μg | 87 | 5.71 (1.746) | 86 | −1.55 (0.196) | −1.11 | −1.62, −0.60 | <0.001 |
| Placebo | 108 | 5.84 (1.730) | 104 | −0.44 (0.178) | — | — | — |

Supplementary Estimand 2

| | | | | | | | |
|---|---|---|---|---|---|---|---|
| All OPN-375 | 172 | 5.63 (1.744) | 169 | −1.50 (0.128) | −0.66 | −1.04, −0.28 | <0.001 |
| OPN-375 372 μg | 85 | 5.54 (1.749) | 83 | −1.46 (0.178) | −0.62 | −1.07, −0.17 | 0.007 |
| OPN-375 186 μg | 87 | 5.71 (1.746) | 86 | −1.53 (0.173) | −0.69 | −1.14, −0.25 | 0.002 |
| Placebo | 108 | 5.84 (1.730) | 105 | −0.84 (0.157) | — | — | — |

Abbreviations:
AECRS = acute exacerbations of chronic rhinosinusitis;
BID = twice daily;
CFB = change from baseline;
CI = confidence interval;
LSM = least squares mean;
LSMD = least squares mean difference (active − placebo);
SD = standard deviation;
SE = standard error

OPN-375 produced improvement compared to placebo in CS patients who did not have polyps in the nasal cavity at baseline who entered the study using standard-delivery nasal steroids. The improvement over placebo was statistically significant for all OPN-375 patients vs placebo irrespective of analytic approach (primary, alternative primary, SE-2 estimand), with the largest treatment difference and with statistical significance for individual dose groups under the alternative primary estimand (Table 16).

TABLE 16
Composite Symptom Scores for Patients Using Standard-Delivery Nasal
Steroid at Trial Entry by Phenotype (Change from Baseline to End of Week 4).

| | | | Week 4 | | | | |
|---|---|---|---|---|---|---|---|
| Treatment | Baseline | | | LSM CFB | | | |
| (BID) | n | Mean (SD) | n | (SE) | LSMD | 95% CI | P-value |

Primary Estimand

| | | | | | | | |
|---|---|---|---|---|---|---|---|
| CS Patients With Nasal Polyps | | | | | | | |
| All OPN-375 | 74 | 5.51 (1.923) | 72 | −1.57 (0.216) | −0.90 | −1.52, −0.29 | 0.004 |
| OPN-375 372 μg | 36 | 5.33 (1.875) | 35 | −1.52 (0.292) | −0.85 | −1.58, 0.12 | 0.023 |
| OPN-375 186 μg | 38 | 5.68 (1.977) | 37 | −1.63 (0.287) | −0.96 | −1.67, −0.25 | 0.009 |
| Placebo | 44 | 5.79 (1.718) | 44 | −0.67 (0.268) | — | — | — |
| CS Patients Without Nasal Polyps | | | | | | | |
| All OPN-375 | 98 | 5.71 (1.601) | 97 | −1.36 (0.168) | −0.54 | −1.06, −0.01 | 0.045 |
| OPN-375 372 μg | 49 | 5.69 (1.652) | 48 | −1.28 (0.238) | −0.45 | −1.07, 0.16 | 0.150 |
| OPN-375 186 μg | 49 | 5.73 (1.564) | 49 | −1.45 (0.235) | −0.62 | −1.24, −0.00 | 0.050 |
| Placebo | 64 | 5.86 (1.751) | 60 | −0.83 (0.212) | — | — | — |

Alternative primary estimand-Intercurrent Events Include Antibiotics for AECRS

| | | | | | | | |
|---|---|---|---|---|---|---|---|
| CS Patients With Nasal Polyps | | | | | | | |
| All OPN-375 | 74 | 5.51 (1.923) | 72 | −1.62 (0.234) | −1.23 | −1.90, −0.56 | <0.001 |
| OPN-375 372 μg | 36 | 5.33 (1.875) | 35 | −1.55 (0.317) | −1.16 | −1.96, −0.37 | 0.004 |
| OPN-375 186 μg | 38 | 5.68 (1.977) | 37 | −1.68 (0.312) | −1.30 | 2.08, −0.52 | 0.001 |
| Placebo | 44 | 5.79 (1.718) | 44 | −0.39 (0.291) | — | — | — |
| CS Patients Without Nasal Polyps | | | | | | | |
| All OPN-375 | 98 | 5.71 (1.601) | 97 | −1.33 (0.183) | −0.88 | −1.45, −0.30 | 0.003 |
| OPN-375 372 μg | 49 | 5.69 (1.652) | 48 | 1.23 (0.259) | −0.77 | −1.44, −0.10 | 0.025 |
| OPN-375 186 μg | 49 | 5.73 (1.564) | 49 | −1.44 (0.256) | −0.98 | −1.65, −0.31 | 0.005 |
| Placebo | 64 | 5.86 (1.751) | 60 | −0.46 (0.231) | — | — | — |

Supplementary Estimand-2

| | | | | | | | |
|---|---|---|---|---|---|---|---|
| CS Patients With Nasal Polyps | | | | | | | |
| All OPN-375 | 74 | 5.51 (1.923) | 72 | −1.59 (0.210) | −0.75 | −1.34, −0.15 | 0.014 |
| OPN-375 372 μg | 36 | 5.33 (1.875) | 35 | −1.53 (0.282) | −0.69 | −1.39, 0.01 | 0.054 |
| OPN-375 186 μg | 38 | 5.68 (1.977) | 37 | −1.64 (0.278) | −0.80 | −1.49, −0.11 | 0.023 |
| Placebo | 44 | 5.79 (1.718) | 43 | −0.84 (0.260) | — | — | — |
| CS Patients Without Nasal Polyps | | | | | | | |
| All OPN-375 | 98 | 5.71 (1.601) | 97 | −1.41 (0.162) | −0.58 | −1.08, −0.08 | 0.023 |
| OPN-375 372 μg | 49 | 5.69 (1.652) | 48 | −1.38 (0.229) | −0.55 | −1.14, 0.04 | 0.068 |
| OPN-375 186 μg | 49 | 5.73 (1.564) | 49 | −1.44 (0.226) | −0.61 | 1.21, −0.02 | 0.042 |
| Placebo | 64 | 5.86 (1.751) | 62 | −0.83 (0.202) | — | — | — |

Abbreviations:
AECRS = acute exacerbations of chronic rhinosinusitis;
AM = morning;
BID = twice daily;
CI = confidence interval;
CS = chronic sinusitis;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (2, 4), treatment-by-week interaction, and continuous covariate: baseline 7-day average score, with baseline score-by-week interaction; treatment-by-subgroup, subgroup-by-week, and treatment-by-subgroup-by-week interaction.

Selected Secondary Endpoints

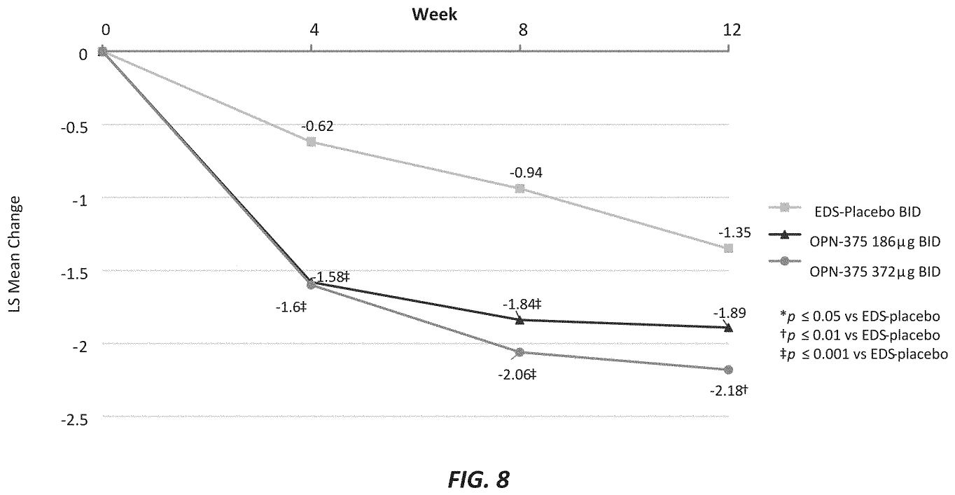

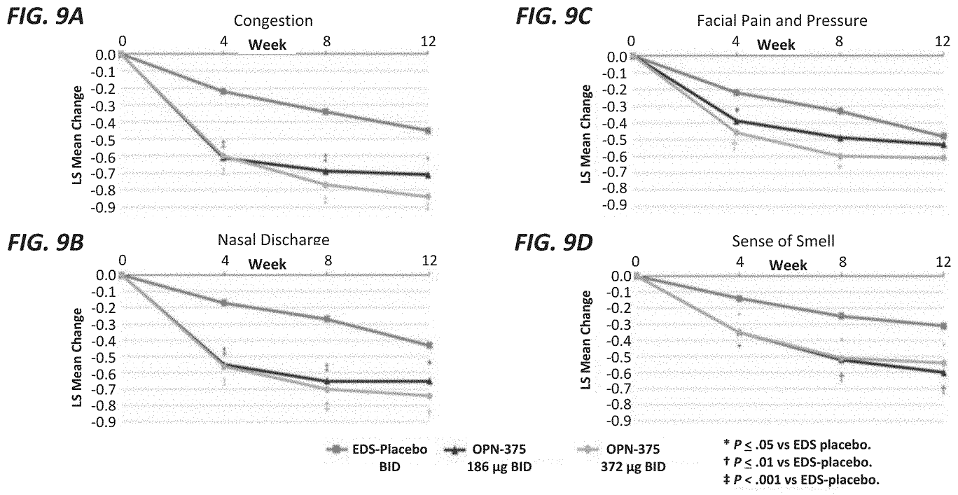

Clinical benefit on symptoms of OPN-375 over placebo comparator was observed through Week 12, which was the full period of data collection via daily symptom reporting diaries. For the FAS, the CSS scores (AM instantaneous) (FIG. 8) and each of the 4 individual DNS (FIGS. 9A-9D) generally decreased/improved numerically compared to baseline in all 3 treatment groups at Weeks 4, 8, and 12, with numerically greater improvement in active treatment groups, indicating that the treatment effect observed using the CSS is not the result of effect on only a single symptom, and with the magnitude of improvement from baseline after the Week 4 primary endpoint in OPN-375 groups being generally similar or slightly improved at timepoints after Week 4. Results for mean AM instantaneous, mean PM instantaneous, mean AM reflective, and mean PM reflective scores by visit for the CSS and individual symptoms for the FAS were similar to those for AM instantaneous scores, demonstrating that the duration of symptomatic benefit was sustained for the 12-hour dosing interval. Mean scores of subject assessments of both instantaneous and reflective nasal symptoms obtained AM and PM decreased/improved in all 3 treatment groups over the 12 weeks of double-blind treatment.

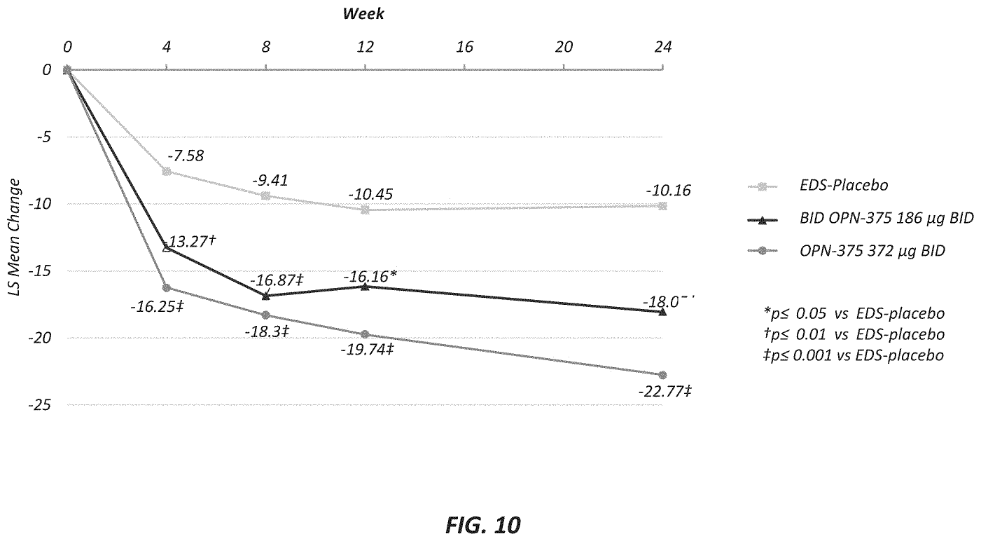

Results for the SNOT-22 total score (collected through Week 24/ET) were supportive of the findings on CSS and individual symptom data collected in study diaries (collected through Week 12) (FIG. 10). Active treatment groups improved significantly more than the placebo comparator group, reaching nominal statistical significance at every time point for both doses when using either the primary estimand or the alternative primary estimand (Table 17). As with other outcome measures, the magnitude of improvement with active over placebo was larger with the alternative primary estimand. The numerical LS mean changes from baseline at all timepoints for each OPN-375 group were similar to or larger than the MCID for each OPN-375 dose group compared to placebo at all timepoints (Table 17). Regarding domain scores of the SNOT-22, nominal statistically significant treatment differences in favor of each active treatment group were found for the rhinologic symptoms domain and the sleep dysfunction domain scores at every timepoint. The treatment differences for the extra-nasal rhinologic symptoms domain scores at every timepoint were nominally statistically significant in favor of both OPN-375 groups through Week 8 and the 372 μg OPN-375 group through Week 24/ET.

TABLE 17
Sinonasal Outcome Test-22 Total Score Change Over Time.

| OPN-375 | Week 4 | | Week 8 | | Week 12 | | Week 24/ET | |
|---|---|---|---|---|---|---|---|---|
| Dose | 186 μg | 372 μg | 186 μg | 372 μg | 186 μg | 372 μg | 186 μg | 372 μg |

Primary Estimand

| | | | | | | | | |
|---|---|---|---|---|---|---|---|---|
| LSM | −13.27 | −16.25 | −16.87 | −18.30 | −16.16 | −19.74 | −18.05 | −22.77 |
| CFB | | | | | | | | |
| LSMD | −5.70 | −8.68 | −7.46 | −8.89 | −5.71 | −9.29 | −7.89 | −12.61 |
| 95% CI | −10.03, | −13.05, | −11.90, | −13.35, | −10.58, | −14.18, | −12.57, | −17.32, |
| | −1.37 | −4.31 | −3.03 | −4.44 | −0.83 | −4.41 | −3.21 | −7.90 |
| P-value | 0.010 | <0.001 | 0.001 | <0.001 | 0.022 | <0.001 | 0.001 | <0.001 |

Alternative primary estimand-Intercurrent Events Include Antibiotics for AECRS

| | | | | | | | | |
|---|---|---|---|---|---|---|---|---|
| LSM | −12.92 | −16.08 | −16.00 | −18.00 | −15.20 | −18.60 | −13.30 | −20.12 |
| CFB | | | | | | | | |
| LSMD | −7.84 | −11.00 | −10.41 | −12.40 | −8.73 | −12.12 | −8.30 | −15.12 |
| 95% CI | −12.35, | −15.57, | −15.26, | −17.28, | −14.14, | −17.55, | −13.92, | −20.77, |
| | −3.33 | −6.44 | −5.56 | −7.53 | −3.32 | −6.70 | −2.69 | −9.47 |
| P-value | <0.001 | <0.001 | <0.001 | <0.001 | 0.002 | <0.001 | 0.004 | <0.001 |

Abbreviations:
AECRS = acute exacerbations of chronic rhinosinusitis;
CFB = change from baseline;
CI = confidence interval;
ET = early termination;
LSM = least squares mean;
LSMD = least squares mean difference (active − placebo)

AECRS is a very important outcome for patients and physicians. AECRS incidence in CRS patients drives both decreased quality of life and a large degree of healthcare utilization. Roughly 10 million patient visits per year for AECRS occur in the US; approximately 70% of these visits result in prescription of an antibiotic; and AECRS is a major driver of adult antibiotic use in the US (Smith et al., 2013). In this study, all but 3 of the observed acute exacerbation events were associated with use of antibiotics.

It should be recognized that major societal changes in all countries involved in this trial in response to the coronavirus disease 2019 (COVID-19) pandemic while the trial was ongoing likely reduced the background (placebo) incidence of AECRS. Reductions in incidence of many acute upper respiratory infections were observed with obvious contributing factors including extended periods of “lockdowns”, mandatory masking in public, reduced seeking of healthcare for reasons unrelated to COVID-19, and other factors.

Despite the likely reduction in environmental risk of acute exacerbations during the period of this study, active treatment with OPN-375 was found to produce a large magnitude reduction in the incidence of AECRS compared to placebo. In the all OPN-375 treatment group, 25 of 217 (11.5%) subjects had 27 AECRS, and in the placebo group, 21 of 110 (19.1%) subjects had 28 AECRS. The incidence rate ratio (IRR) for AECRS with OPN-375 (combined doses) versus placebo was 0.416, reflecting a reduction in acute exacerbations of approximately 58% (nominal P=0.005). The incidence of AECRS was lower in the high dose group (8 [6.5%] subjects with 9 exacerbations) than the low dose group (17 [15.5%] subjects with 18 exacerbations). Specifically, in the high dose group, there was a 70% reduction (IRR=0.295; nominal P=0.004) and, in the low dose group, a 41% reduction (IRR=0.586; nominal P=0.120).

Kaplan-Meier analysis of time to first acute exacerbation showed separation between the 3 treatment groups, with the OPN-375 groups consistently having a larger proportion of subjects without exacerbations over 24 weeks of treatment. The largest treatment benefit was observed in the 372 μg OPN-375 dose group. The log rank chi square p-value was 0.005 for the high dose group and 0.031 for the combined OPN-375 group, reaching nominal statistical significance.

Sleep quality was assessed using the Pittsburgh Sleep Quality Index (PSQI) at baseline, Week 12, and Week 24/ET of the double-blind treatment phase. Mean PSQI Global Scores decreased (i.e., improved) numerically from baseline to Week 24/ET in all 3 treatment groups with larger improvement in the active treatment groups. However, global score treatment differences (for each active group minus placebo) did not reach nominal statistical significance for either dose of OPN-375, albeit nominal statistical significance of treatment differences was reached for a few of the component scores. Each of the Subjective Sleep Quality component and the Sleep Disturbance component scores were nominally statistically significantly in favor of active treatment (372 μg OPN-375 group) at the end of the double-blind treatment phase (Week 24/ET).

The Patient Global Impression of Change (PGIC), a direct patient report of their perception of change in their overall symptom burden, was assessed at Week 4 and Week 24/ET of the double-blind treatment phase. For both active treatment groups, the likelihood of improvement as measured by odds ratio (vs the placebo group) was nominally statistically significant at both Week 4 and Week 24/ET with odds ratios of 2.6 (low dose) and 3.1 (high dose) for improvement at Week 24/ET (Table 18). Using a narrower definition that required a high degree of improvement (i.e., patient report of being “very much” improved or “much” improved), the odds ratios remained nominally statistically significant for both active groups at both timepoints. From a global patient-reported perspective, there was a clear perception of improvement in symptoms for more subjects receiving OPN-375 than subjects receiving placebo.

TABLE 18
Patient Global Impression of Change at Week 4 and Week 24/ET.

| | | | OPN 375 | OPN 375 |
|---|---|---|---|---|
| | | Placebo | 186 μg | 372 μg |
| Time Point | Category | (N = 110) | (N = 110) | (N = 107) |
| Week 4 | N | 104 | 109 | 103 |
| | Improved | 57 (54.8) | 84 (77.1) | 77 (74.8) |
| | Very much improved | 0 | 8 (7.3) | 11 (10.7) |
| | Much improved | 7 (6.7) | 29 (26.6) | 28 (27.2) |
| | Minimally improved | 50 (48.1) | 47 (43.1) | 38 (36.9) |
| | Not Improved | 47 (45.2) | 25 (22.9) | 26 (25.2) |
| | Odds Ratio (Improved) | — | 2.80 | 2.50 |
| | P-value | — | <0.001 | 0.003 |
| Week 24/ET | N | 94 | 99 | 98 |
| | Improved | 56 (59.6) | 78 (78.8) | 80 (81.6) |
| | Very much improved | 4 (4.3) | 10 (10.1) | 15 (15.3) |
| | Much improved | 2 (21.3) | 34 (34.3) | 38 (38.8) |
| | Minimally improved | 32 (34.0) | 34 (34.3) | 27 (27.6) |
| | Not Improved | 38/94 (40.4) | 21/99 (21.2) | 18/98 (18.4) |
| | Odds Ratio (Improved) | — | 2.61 | 3.10 |
| | P-value | — | 0.004 | <0.001 |

Abbreviations:
ET = early termination;
MMRM = mixed model for repeated measures
Note:
The denominator for each visit is the number of subjects with an assessment at the visit. Inferential statistics are based on a logistic MMRM including categorical effects for previous sinus surgery, nasal polyp status, treatment, week (4, 24/ET), and treatment-by-week interaction.

It was important to show a reduction in symptoms in the subgroup of patients without nasal polyps in order to demonstrate that the treatment effect of OPN-375 in this study could not be “merely due to reduction in polyp size.”

Treatment with OPN-375, at both tested doses, reduced symptoms in chronic sinusitis patients of both phenotypes. Symptom improvement as measured by composite symptom score (co-primary endpoint) was generally similar in magnitude for patients entering Study A either with or without NP. This finding is robust based on not only the primary estimand, but also on the alternative primary estimand (including both use of antibiotics for AECRS and use of systemic corticosteroids as intercurrent events), as well as when using the SE-2 estimand: symptom improvement for CS patients with both phenotypes reached nominal statistical significance compared to placebo in all cases (Table 19).

Recall that the alternative primary estimand included the use of antibiotics for AECRS as an intercurrent event, in order to better reflect the fact that not only use of systemic corticosteroids but also an acute exacerbation treated with antibiotics can reflect treatment failure. The alternative primary estimand assigned a poor symptom score when such events occurred prior to the Week 4 assessment. The alternative primary estimand showed treatment differences for both subjects with and without NP in favor of OPN-375, and the numerical size of the differences was greater than the differences observed using the primary estimand and SE-2, though, as noted, all reached nominal statistical significance. The increased numerical difference between active and placebo when using the alternative primary estimand is a result of reduction in placebo response when using this estimand because most subjects who failed treatment and experienced an AECRS requiring an antibiotic were in the placebo group.

TABLE 19
Change in Composite Nasal Symptom Score by Phenotype (LS Mean,
Baseline to End of Week 4).

| | | | Week 4 | | | | |
|---|---|---|---|---|---|---|---|
| Population | Baseline | | | LSM (SE) | | | |
| Treatment (BID) | n | Mean (SD) | n | CFB | LSMD | 95% CI | P-value |

Primary Estimand

| | | | | | | | |
|---|---|---|---|---|---|---|---|
| CS Patients With Nasal Polyps | | | | | | | |
| All OPN-375 | 136 | 5.33 (1.804) | 134 | 1.62 (0.144) | −1.06 | −1.55, −0.57 | <0.001 |
| OPN-375 372 μg | 67 | 5.28 (1.806) | 66 | 1.66 (0.204) | −1.10 | −1.67, −0.54 | <0.001 |
| OPN-375 186 μg | 69 | 5.37 (1.814) | 68 | −1.58 (0.201) | −1.02 | −1.58, −0.46 | <0.001 |
| Placebo | 69 | 5.81 (1.841) | 68 | −0.56 (0.201) | — | — | — |
| CS Patients Without Nasal Polyps | | | | | | | |
| All OPN-375 | 81 | 5.65 (1.831) | 80 | −1.47 (0.188) | −0.80 | −1.43, −0.17 | 0.013 |
| OPN-375 372 μg | 40 | 5.80 (1.857) | 40 | −1.44 (0.265) | −0.77 | −1.50, −0.04 | 0.038 |
| OPN-375 186 μg | 41 | 5.51 (1.817) | 40 | −1.50 (0.264) | −0.83 | −1.56, −0.10 | 0.025 |
| Placebo | 41 | 5.69 (1.678) | 40 | −0.67 (0.263) | — | — | — |

Alternative primary estimand-Intercurrent Events Include Antibiotics for AECRS

| | | | | | | | |
|---|---|---|---|---|---|---|---|
| CS Patients With Nasal Polyps | | | | | | | |
| All OPN-375 | 136 | 5.33 (1.804) | 134 | −1.59 (0.157) | −1.25 | −1.79, −0.72 | <0.001 |
| OPN-375 372 μg | 67 | 5.28 (1.806) | 66 | −1.67 (0.223) | −1.33 | −1.95, −0.71 | <0.001 |
| OPN-375 186 μg | 69 | 5.37 (1.814) | 68 | 1.51 (0.220) | −1.17 | −1.79, −0.56 | <0.001 |
| Placebo | 69 | 5.81 (1.841) | 68 | −0.34 (0.220) | — | — | — |
| CS Patients Without Nasal Polyps | | | | | | | |
| All OPN-375 | 81 | 5.65 (1.831) | 80 | −1.43 (0.206) | −1.16 | −1.85, −0.47 | <0.001 |
| OPN-375 372 μg | 40 | 5.80 (1.857) | 40 | −1.38 (0.290) | −1.11 | −1.91, −0.32 | 0.006 |
| OPN-375 186 μg | 41 | 5.51 (1.817) | 40 | −1.48 (0.289) | −1.21 | −2.01, −0.42 | 0.003 |
| Placebo | 41 | 5.69 (1.678) | 40 | −0.27 (0.287) | — | — | — |

Supplementary Estimand-2

| | | | | | | | |
|---|---|---|---|---|---|---|---|
| CS Patients With Nasal Polyps | | | | | | | |
| All OPN-375 | 136 | 5.33 (1.804) | 134 | −1.62 (0.136) | −0.95 | −1.42, −0.49 | <0.001 |
| OPN-375 372 μg | 67 | 5.28 (1.806) | 66 | −1.66 (0.194) | −0.99 | −1.53, −0.46 | <0.001 |
| OPN-375 186 μg | 69 | 5.37 (1.814) | 68 | −1.58 (0.191) | −0.91 | −1.44, −0.38 | <0.001 |
| Placebo | 69 | 5.81 (1.841) | 67 | −0.67 (0.191) | — | — | — |
| CS Patients Without Nasal Polyps | | | | | | | |
| All OPN-375 | 8 | 5.65 (1.831) | 81 | −1.57 (0.177) | −0.92 | −1.51, −0.32 | 0.003 |
| OPN-375 372 μg | 40 | 5.80 (1.857) | 40 | −1.56 (0.252) | −0.91 | −1.60, −0.22 | 0.010 |
| OPN-375 186 μg | 41 | 5.51 (1.817) | 41 | −1.58 (0.247) | −0.93 | −1.61, −0.24 | 0.008 |
| Placebo | 41 | 5.69 (1.678) | 41 | −0.65 (0.248) | — | — | — |

Abbreviations:
AECRS = acute exacerbations of chronic rhinosinusitis;
AM = morning;
BID = twice daily;
CFB = change from baseline;
CI = confidence interval;
CS = chronic sinusitis;
CSR = clinical study report;
LS = least squares;
LSM = least squares mean;
LSMD = least squares mean difference (active − placebo);
SD = standard deviation;
SE = standard error

Analyses of change over time for timepoints beyond the primary endpoint (i.e., Weeks 8 and 12 for symptoms, Week 24 for SNOT-22) were also performed by phenotype to inform whether treatment response was maintained after the primary analysis at Week 4. For CS patients without nasal polyps, the magnitude of improvement from baseline in composite symptom score with OPN-375 treatment remains similar or slightly increases over time past the primary endpoint at 4 weeks. Under the alternative primary estimand, the placebo-subtracted improvements observed at Week 4 continued to be nominally statistically significant at the high dose through Week 8 and the difference at Week 12 remained consistent (Table 20).

Persistence of response over time by phenotype in this study may also be informed by the secondary outcome measure SNOT-22, which was obtained at timepoints through Week 24. As with CSS, the magnitude of improvement from baseline for CS patients without nasal polyps that was observed at Week 4 persisted, in this case through Week 24 (Table 21). At all timepoints through Week 24, the improvement from baseline observed with OPN-375 among CS patients without nasal polyps was greater than the minimally clinically important difference for the SNOT-22 measurement instrument (i.e., 8.9 points).

TABLE 20
Composite Symptom Score Change Over Time by Phenotype.

| | Week 4 | | Week 8 | | Week 12 | |
|---|---|---|---|---|---|---|
| OPN-375 Dose | 186 μg | 372 μg | 186 μg | 372 μg | 186 μg | 372 μg |

Primary Estimand

| | | | | | | |
|---|---|---|---|---|---|---|
| CS Patients With Nasal Polyps | | | | | | |
| LSM CFB | −1.58 | −1.66 | −1.95 | −2.26 | −2.00 | −2.34 |
| LSMD | −1.02 | −1.10 | −1.17 | −1.48 | −0.79 | −1.12 |
| 95% CI | −1.58, | −1.67, | −1.84, | −2.16, | −1.50, | −1.85, |
| | −0.46 | −0.54 | −0.50 | −0.80 | −0.07 | −0.40 |
| P-value | <0.001 | <0.001 | <0.001 | <0.001 | 0.031 | 0.003 |
| CS Patients Without Nasal Polyps | | | | | | |
| LSM CFB | −1.50 | −1.44 | −1.61 | −1.74 | −1.64 | −1.91 |
| LSMD | −0.83 | −0.77 | −0.39 | −0.52 | −0.07 | −0.34 |
| 95% CI | −1.56, | −1.50 | −1.26, | −1.39, | −1.00, | −1.27, |
| | −0.10 | −0.04 | 0.48 | 0.35 | 0.87 | 0.59 |
| P-value | 0.025 | 0.038 | 0.375 | 0.242 | 0.890 | 0.470 |

Alternative primary estimand-Intercurrent Events Include Antibiotics for AECRS

| | | | | | | |
|---|---|---|---|---|---|---|
| CS Patients With Nasal Polyps | | | | | | |
| LSM CFB | −1.51 | −1.67 | −1.82 | −2.26 | −1.88 | −2.28 |
| LSMD | −1.17 | −1.33 | −1.36 | −1.81 | −1.03 | −1.43 |
| 95% CI | −1.79, | −1.95, | −2.10, | −2.56, | −1.85, | −2.26, |
| | −0.56 | −0.71 | −0.62 | −1.05 | −0.22 | −0.61 |
| P-value | <0.001 | <0.001 | <0.001 | <0.001 | 0.013 | <0.001 |
| CS Patients Without Nasal Polyps | | | | | | |
| LSM CFB | −1.48 | −1.38 | −1.50 | −1.62 | −1.20 | −1.65 |
| LSMD | −1.21 | −1.11 | −0.85 | −0.97 | −0.50 | −0.95 |
| 95% CI | −2.01, | −1.91, | −1.82, | −1.94, | −1.56, | −2.00, |
| | −0.42 | −0.32 | 0.11 | −0.00 | 0.56 | 0.11 |
| P-value | 0.003 | 0.006 | 0.084 | 0.049 | 0.352 | 0.078 |

Abbreviations:
AECRS = acute exacerbations of chronic rhinosinusitis;
CFB = change from baseline;
CI = confidence interval;
CS = chronic sinusitis;
ET = early termination;
LSM = least squares mean;
LSMD = least squares mean difference (active − placebo)

TABLE 21
Sinonasal Outcome Test-22 Total Score by Phenotype.

| | Week 4 | | Week 8 | | Week 12 | | Week 24/ET | |
|---|---|---|---|---|---|---|---|---|
| OPN-375 Dose | 186 μg | 372 μg | 186 μg | 372 μg | 186 μg | 372 μg | 186 μg | 372 μg |

Primary Estimand

| | | | | | | | | |
|---|---|---|---|---|---|---|---|---|
| CS Patients With Nasal Polyps | | | | | | | | |
| LSM CFB | −11.91 | −15.56 | −16.98 | −18.47 | −16.20 | −20.90 | −19.26 | −23.23 |
| LSMD | −7.65 | −11.31 | −9.76 | −11.26 | −7.83 | −12.53 | −12.71 | −16.68 |
| 95% CI | −13.09, | −16.81, | −15.31, | −16.84, | −13.92, | −18.64, | −18.53, | −22.56, |
| | −2.21 | −5.81 | −4.21 | −5.67 | −1.74 | −6.43 | −6.88 | −10.80 |
| P-value | 0.006 | <0.001 | <0.001 | <0.001 | 0.012 | <0.001 | <0.001 | <0.001 |
| CS Patients Without Nasal Polyps | | | | | | | | |
| LSM CFB | −14.43 | −16.37 | −15.48 | −17.02 | −14.89 | −16.87 | −14.79 | −21.07 |
| LSMD | −2.15 | −4.08 | −3.32 | −4.86 | −1.89 | −3.86 | 0.46 | −5.81 |
| 95% CI | −9.26, | −11.27, | −10.66, | −12.24, | −9.92, | −11.90, | −7.17, | −13.46, |
| | 4.97 | 3.11 | 4.03 | 2.52 | 6.14 | 4.17 | 8.10 | 1.84 |
| P-value | 0.553 | 0.265 | 0.375 | 0.196 | 0.644 | 0.345 | 0.905 | 0.136 |

Alternative primary estimand-Intercurrent Events Include Antibiotics for AECRS

| | | | | | | | | |
|---|---|---|---|---|---|---|---|---|
| CS Patients With Nasal Polyps | | | | | | | | |
| LSM CFB | −11.49 | −15.56 | −16.36 | −18.45 | −15.94 | −20.15 | −14.94 | −20.85 |
| LSMD | −9.54 | −13.61 | −12.70 | −14.79 | −10.81 | −15.03 | −12.02 | −17.92 |
| 95% CI | −15.22, | −19.34, | −18.77, | −20.90, | −17.57, | −21.81, | −19.05, | −25.01, |
| | −3.87 | −7.87 | −6.62 | −8.67 | −4.06 | −8.24 | −5.00 | −10.84 |
| P-value | 0.001 | <0.001 | <0.001 | <0.001 | 0.002 | <0.001 | <0.001 | <0.001 |
| CS Patients Without Nasal Polyps | | | | | | | | |
| LSM CFB | −14.36 | −16.07 | −14.39 | −16.42 | −12.92 | −15.22 | −9.45 | −18.09 |
| LSMD | −4.76 | −6.47 | −6.33 | −8.36 | −4.98 | −7.28 | −178 | −10.42 |
| 95% CI | 12.18, | −13.96, | −14.34, | 16.41, | −13.87, | −16.18, | −11.00, | −19.65, |
| | 2.66 | 1.03 | 1.69 | −0.30 | 3.91 | 1.62 | 7.44 | −1.18 |
| P-value | 0.208 | 0.091 | 0.121 | 0.042 | 0.271 | 0.109 | 0.704 | 0.027 |

Abbreviations:
AECRS = acute exacerbations of chronic rhinosinusitis;
CFB = change from baseline;
CI = confidence interval;
CS = chronic sinusitis;
ET = early termination;
LSM = least squares mean;
LSMD = least squares mean difference (active − placebo)

Given that OPN-375 is an intranasally delivered treatment previously demonstrated to improve nasal polyps, the purpose of assessing symptom response in patients without NP was to assure that efficacy in Study A was not solely due to treatment of polyps in the nasal cavity. This rationale is further supported by the fact that the same symptoms, measured identically, are used to assess symptom response in nasal polyps (an indication manifesting in the nasal cavity) and in chronic sinusitis (a disease that by definition affects the paranasal sinuses), creating risk that nasal and sinus treatment effects could be conflated.

This rationale does not apply to the measurement of change in opacified volume in the sinus cavities, because APOV is by definition an outcome measure that is highly specific to the sinus cavities and cannot be conflated with outcomes in the nasal cavity. Demonstration of biological effect inside sinus cavities affected by disease can be achieved in the entire population enrolled in Study A. Accordingly, unlike symptoms, there is no primary hypothesis to be addressed by analysis of change in sinus opacification in subgroups by phenotype. Nevertheless, an evaluation of change in sinus opacification for chronic sinusitis phenotypic sub-groups with and without nasal polyps was performed.

At baseline, sinus opacification was slightly different in subpopulations with and without polyps in the nasal cavity (˜10% greater baseline opacification in CS patients who also had nasal polyps). Under the primary estimand, there was a similar improvement from baseline in sinus opacification (APOV) for chronic sinusitis subjects with or without polyps in the nasal cavity who were treated with OPN-375 (with polyps: −5.32; without polyps: −5.71) (Table 22). However, there was a sharp difference in the change from baseline for placebo subjects with or without nasal polyps (with polyps: +1.29; without polyps: −5.50). The material improvement from baseline in subjects without nasal polyps (vs worsening from baseline in CS subjects with NP) is both clinically unexpected and different from the placebo group findings in Study B. As a consequence of this difference in placebo response in the two sub-populations, the placebo-subtracted difference is nominally statistically significant in the CS subpopulation with nasal polyps but not in the subpopulation without nasal polyps. The magnitude of improvement in CS subjects without nasal polyps was highest in subjects receiving OPN-375 high dose. Although there are small numerical differences, these findings are generally the same when using the SE-2 estimand.

An extensive evaluation of patients was undertaken to attempt to identify factors that could explain the differences observed in the placebo sub-groups with and without NP. For example, it was hypothesized that acute viral infection at study entry in a small number of subjects may have resolved spontaneously during the study, or that differential use of concomitant medications may have occurred, or that there may have been different baseline characteristics. Unfortunately, no clear explanation for the placebo findings was identified. The most likely remaining explanation is simply that the significantly reduced subgroup sample size enabled a small number of extreme outliers to have a large impact on mean changes in the placebo-treated non-polyp group.

The alternative primary estimand was utilized in order to reflect the fact that treatment failure can be indicated not only by patients requiring a systemic corticosteroid, but also by patients experiencing an AECRS that is treated with an antibiotic. Under the alternative primary estimand, in which use of an antibiotic for AECRS was considered an intercurrent event, there was a substantial difference in the mean change from baseline calculated for the non-polyp subjects receiving placebo compared to the other estimands. Because the majority of AECRS events occurred in the placebo group, the “poor score” composite strategy of the alternative primary estimand produces a quite different placebo response, changing the placebo response from an unexpected and comparatively large placebo improvement (under primary estimand) to a small placebo worsening (under alternative primary estimand) as might have been clinically expected for untreated patients. Under the alternative primary estimand, mostly as a result of this large effect on placebo response, the drug placebo difference for the high dose is numerically similar to that observed in the overall population using the same alternative primary estimand (CRSsNP high dose: −5.56 vs Total Population: −5.24). In addition, under the alternative primary estimand, given the differences in baseline opacification, the proportional treatment improvement over placebo was nearly identical between high-dose patients with and without nasal polyps (˜9%). Proportional treatment improvement in the high dose groups with or without nasal polyps was also similar under the SE-2 estimand.

TABLE 22
Change in Average Percentages of Opacified Volume by Phenotype
(Baseline to Week 24/ET).

| | | | Week 24/ET | | | | |
|---|---|---|---|---|---|---|---|
| Population | Baseline | | | LSM (SE) | | | |
| Treatment (BID) | n | Mean (SD) | n | CFB | LSMD | 95% CI | P-value |

Primary Estimand

| | | | | | | | |
|---|---|---|---|---|---|---|---|
| CS Patients With Nasal Polyps | | | | | | | |
| All OPN-375 | 136 | 73.14 (17.59) | 126 | −5.32 (1.234) | −6.62 | −10.75, −2.48 | 0.002 |
| OPN-375 372 μg | 67 | 73.90 (16.90) | 63 | −4.64 (1.744) | −5.93 | −10.68, −1.18 | 0.014 |
| OPN-375 186 μg | 69 | 72.41 (18.34) | 63 | −6.01 (1.741) | −7.30 | −12.12, −2.48 | 0.003 |
| Placebo | 69 | 73.10 (21.10) | 62 | 1.29 (1.727) | — | — | — |
| CS Patients Without Nasal Polyps | | | | | | | |
| All OPN-375 | 81 | 61.81 (19.63) | 73 | −5.71 (1.692) | −0.21 | −5.70, 5.27 | 0.939 |
| OPN-375 372 μg | 40 | 60.65 (19.20) | 38 | −7.90 (2.287) | −2.40 | −8.66, 3.85 | 0.451 |
| OPN-375 186 μg | 41 | 62.95 (20.21) | 35 | −3.52 (2.389) | 1.98 | −4.42, 8.38 | 0.545 |
| Placebo | 41 | 61.92 (17.00) | 37 | −5.50 (2.309) | — | — | — |

Alternative primary estimand-Intercurrent Events Include Antibiotics for AECRS

| | | | | | | | |
|---|---|---|---|---|---|---|---|
| CS Patients With Nasal Polyps | | | | | | | |
| All OPN-375 | 136 | 73.14 (17.59) | 128 | −3.32 (1.324) | −7.14 | −11.58, −2.70 | 0.002 |
| OPN-375 372 μg | 67 | 73.90 (16.90) | 63 | −3.14 (1.884) | −6.96 | −12.11, −1.80 | 0.008 |
| OPN-375 186 μg | 69 | 72.41 (18.34) | 65 | −3.51 (1.843) | −7.33 | −12.43, −2.23 | 0.005 |
| Placebo | 69 | 73.10 (21.10) | 62 | 3.82 (1.861) | — | — | — |
| CS Patients Without Nasal Polyps | | | | | | | |
| All OPN-375 | 81 | 61.81 (19.63) | 74 | −1.47 (1.776) | −2.00 | −7.84, 3.84 | 0.502 |
| OPN-375 372 μg | 40 | 60.65 (19.20) | 38 | −5.03 (2.470) | −5.56 | −12.23, 1.11 | 0.102 |
| OPN-375 186 μg | 41 | 62.95 (20.21) | 36 | 2.08 (2.501) | 1.56 | −5.29, 8.41 | 0.655 |
| Placebo | 41 | 61.92 (17.00) | 38 | 0.52 (2.455) | — | — | — |

Supplementary Estimand-2

| | | | | | | | |
|---|---|---|---|---|---|---|---|
| CS Patients With Nasal Polyps | | | | | | | |
| All OPN-375 | 136 | 73.14 (17.59) | 130 | −5.64 (1.269) | −5.61 | −9.91, −1.32 | 0.010 |
| OPN-375 372 μg | 67 | 73.90 (16.90) | 66 | −4.61 (1.788) | −4.58 | −9.51, 0.34 | 0.068 |
| OPN-375 186 μg | 69 | 72.41 (18.34) | 64 | −6.67 (1.800) | −6.64 | −11.65, −1.64 | 0.009 |
| Placebo | 69 | 73.10 (21.10) | 63 | −0.02 (1.796) | — | — | — |
| CS Patients Without Nasal Polyps | | | | | | | |
| All OPN-375 | 81 | 61.81 (19.63) | 77 | −7.09 (1.682) | −1.81 | −7.27, 3.66 | 0.517 |
| OPN-375 372 μg | 40 | 60.65 (19.20) | 39 | −8.44 (2.366) | −3.16 | −9.50, 3.18 | 0.329 |
| OPN-375 186 μg | 41 | 62.95 (20.21) | 38 | −5.74 (2.343) | −0.45 | −6.81, 5.90 | 0.889 |
| Placebo | 41 | 61.92 (17.00) | 41 | −5.28 (2.298) | — | — | — |

Abbreviations:
AECRS = acute exacerbations of chronic rhinosinusitis;
BID = twice daily;
CFB = change from baseline;
CI = confidence interval;
CS = chronic sinusitis;
CSR = clinical study report;
ET = early termination;
LSM = least squares mean;
LSMD = least squares mean difference (active − placebo);
SD = standard deviation;
SE = standard error

Adverse Events

TEAEs were reported for 49.5% of OPN-375 recipients (51.4% of subjects in the 186 μg group and 47.7% of subject in the 372 μg group) and 51.8% of placebo recipients (irrespective of nasal polyps). In OPN-375 recipients, epistaxis was the most frequently reported TEAE during the double-blind treatment phase. Overall, the TEAE coded as “epistaxis” was reported for 5 (4.5%), 13 (11.9%), and 1 (0.9%) subject(s) in the 186 μg OPN-375, 372 μg OPN-375, and placebo groups, respectively. Epistaxis was the only TEAE reported at a >5% difference in the all OPN-375 group compared with the placebo group. Epistaxis was the most commonly reported TEAE where the investigator considered it treatment related; it was reported in 4 (3.6%), 5 (4.6%), and 1 (0.9%) subject in the 186 μg OPN-375, 372 μg OPN-375, and placebo groups, respectively.

Nine subjects (2.7%) reported a TEAE leading to study treatment discontinuation (2 subjects in the 186 μg group, 3 subjects in the 372 μg group, and 4 subjects in the placebo group). There was no discernible pattern in the TEAEs leading to discontinuation. Severe AEs were reported in 11 subjects (3 subjects in the 186 μg group, 3 subjects in the 372 μg group, and 5 placebo recipients). One severe event (nasal congestion in a placebo recipient) was considered related to study treatment; all other severe events were unrelated. Six subjects had a total of 9 SAEs (1 event in 1 subject in the 186 μg OPN-375 group, 1 event each in 2 subjects in the 372 μg OPN-375 group, and 6 events in 3 placebo recipients). No SAE was fatal, and none were considered related to study treatment.

TEAEs were reported for 51.9% of OPN-375 recipients (58.5% of subjects in the 186 μg group and 45.0% of subjects in the 372 μg group) and 48.8% of placebo recipients (subjects without nasal polyps at baseline). Epistaxis was the only TEAE reported at a higher incidence in the combined OPN-375 group (7.4%) than in the placebo group (0%) for which there was ≥5%, difference between these groups. Overall, epistaxis was experienced by 3 (7.3%), 3 (7.5%), and 0 subjects in the 186 μg OPN-375, 372 μg OPN-375, and placebo groups, respectively. Epistaxis was considered treatment related in 3 (7.3%) of subjects in the 186 μg OPN-375 group but was not considered treatment related for any subjects in the placebo or 372 μg OPN-375 groups. Severe AEs were reported in 5 subjects (2 subjects in the 186 μg group, 1 subjects in the 372 μg group, and 2 placebo recipients). Nasal examination findings considered clinically significant by the investigator were either medical history/baseline findings (pretreatment nasal examination) or TEAEs (for-cause unscheduled and/or Week 24/ET nasal examination).

A total of 27 subjects (3 subjects in the placebo group, 11 subjects in the 186 μg OPN-375 group, and 13 subjects in the 372 μg OPN-375 group) had a posttreatment finding of bleeding on at least 1 posttreatment nasal examination (i.e., examination at Week 4, Week 12, Week 24/ET and/or an unscheduled visit). Epistaxis was also reported as a TEAE for 10 of the 26 subjects who had a posttreatment nasal examination finding of nonactive bleeding (0 in the placebo group, 3 in the 186 μg OPN-375 group, and 8 in the 372 μg OPN-375 group). The majority of these subjects (9 of 11) experienced TEAEs that were only mild in severity, while 2 subjects experienced at least 1 moderate TEAE of epistaxis; none of the TEAEs were severe.

A total of 12 CS subjects without nasal polyps (3 subjects in the placebo group, 3 subjects in the 186 μg OPN-375 group, and 6 subjects in the 372 μg OPN-375 group) had a finding of bleeding on at least 1 posttreatment nasal examination (i.e., examination at Week 4, Week 12, Week 24/ET and/or an unscheduled visit). None of the findings represented active bleeding. Of the 12 subjects who had at least 1 posttreatment finding of nonactive bleeding, epistaxis was also reported as a TEAE for 5 subjects (0 in the placebo group, 2 in the 186 μg OPN-375 group, and 3 in the 372 μg OPN-375 group). The majority of these subjects (4 of 5) experienced TEAEs that were mild in severity, while 1 subject experienced a moderate TEAE of epistaxis; none of the TEAEs were severe.

Septal erosion was present in 1 subject (0.9%) in the 186 μg OPN-375 group and 2 subjects (1.8%) in the placebo group at screening (irrespective of nasal polyps). It was present in 3 subjects (2.8%) in the 186 μg OPN-375 and 3 subjects (2.9%) in the 372 μg OPN-375 group at Week 24/ET. Septal erosion was not reported in any subject at screening (patients without nasal polyps). At Week 24/ET, it was observed in 3 subjects (7.7%) in the 186 μg OPN 375 and 1 subject (2.6%) in the 372 μg OPN-375 group. Ulceration/erosion in areas other than the nasal septum was present in 1 subject (0.9%) in the placebo group at screening (irrespective of nasal polyps). It was present in 1 subject (1.0%) in the placebo group, no subjects in the 186 μg OPN-375 group, and 2 subjects (1.9%) in the 372 μg OPN-375 group at Week 24/ET. Septal erosion was not reported in any subject at screening (patients without nasal polyps). At Week 24/ET, it was observed in 3 subjects (7.7%) in the 186 μg OPN 375 and 1 subject (2.6%) in the 372 μg OPN-375 group. Mucosal candidiasis was not present in any subjects at any time during the study.

Except for events coded as epistaxis, there were no obvious between-group nasal exam differences at baseline or any other timepoint. However, the numbers of subjects with such findings were low in all treatment groups at all timepoints. Cataracts were reported in 7.2%, 4.6%, and 2.7% of subjects in the 186 μg OPN-375, 372 μg OPN-375, and placebo groups, respectively (irrespective of nasal polyps). The incidence of new cataracts overall was not different between subjects receiving OPN-375 and placebo (0%, 2.8%, and 0.9% of subjects in the 186 μg OPN-375, 372 μg OPN-375, and placebo groups, respectively). For all subjects with chronic sinusitis (irrespective of nasal polyps) and for the subjects without nasal polyps, there were no clinically relevant differences in vital signs (BP and pulse) observed in this study between the subjects in the OPN-375 treatment groups and the subjects in the placebo group. For all subjects with chronic sinusitis (irrespective of nasal polyps) and for the subjects without nasal polyps, there were no obvious differences in concomitant medication use across treatment groups.

Discussion

Treatment with OPN-375 (186 or 372 μg BID) significantly improved symptoms and objective evidence of disease in patients with chronic sinusitis. Statistically significant and clinically meaningful improvement compared to a placebo comparator was demonstrated in pre-specified co-primary subjective (symptom relief at Week 4) and objective (decreased opacity in sinus cavities at Week 24/ET) endpoints. Treatment differences on these primary measures of efficacy were robust to analytic approach.

The demonstration of robust statistically significant benefits on both CSS and APOV provides strong evidence that OPN-375 improves the symptoms and sinus opacification for subjects with CS. Importantly, the improvement can be determined to be clinically meaningful—and broadly affecting the manifestations of the disease—as directly measured by patient-reported outcomes including individual symptom scores, disease-specific quality of life (SNOT-22), sleep (PSQI), and a global patient-reported assessment of disease (PGIC).

OPN-375 produced significantly more improvement than placebo comparator in sinus opacification. Improvement in CT may not be considered clinically important, since the primary goal of treatment is defined in all published treatment guidelines as improvement in symptoms and quality of life, and CT scans are not routinely performed to assess treatment response. However, reduced intra-sinus opacification is evidence of treatment effect on sinus inflammation, and this trial demonstrates topical drug activity extending beyond the nasal cavity into the sinuses themselves. In the case of this trial, the intent of intra-sinus imaging was to demonstrate treatment effect on intra-sinus disease, and not simply on the nasal symptoms accompanying sinus disease, in the context of a topical medication previously known to be effective in treating nasal disease.

As discussed above, symptom outcomes due to treatment of polyps in the nasal cavity could be conflated with symptom outcomes due to treatment of the sinus cavities because symptoms are measured identically (by CSS) in both phenotypes. Therefore, it is useful to analyze symptoms by phenotypic subgroup. Importantly, OPN-375 produced significantly more symptom improvement than placebo comparator in CS patients either with or without nasal polyps, showing that treatment benefit cannot be attributed solely to treatment of NP. In contrast with symptom assessment, CT scans are highly specific to the anatomic region assessed and there is no risk of confusing a treatment effect on NP (in the nasal cavity) with a treatment effect on sinus disease (in the sinus cavity). Therefore, data from all enrolled patients is directly relevant to the question of whether or not a treatment effect is occurring in the sinus cavities, and, in the primary analysis of the full chronic sinusitis population, all with proven intra-sinus disease at baseline, OPN-375 produced significantly more improvement than the placebo comparator in sinus opacification.

Acute symptom exacerbation episodes drive harm to quality of life, increased care, and escalation of care (e.g., to surgery) and, as with other respiratory diseases, are clinically important. OPN-375 produced a substantial reduction in the incidence of AECRS compared to patients receiving placebo. Reduction in AECRS is highly clinically meaningful for individuals, as reducing frequent use of antibiotics may reduce risks such as disruption of normal microbiomes in gut or nose, adverse drug reactions, and other complications (e.g., Clostridium difficile).

Efficacy results on an array of secondary measures support the conclusions established with the co-primary outcome measures. The change from baseline observed on disease-specific quality of life (SNOT-22) with OPN-375 was not only rapid (statistically significantly different than placebo beginning at Week 4, the first measurement timepoint in this study) but substantially greater than the MCID (18- and 22-point improvements for treatment with the low and high doses, respectively) and similar to the magnitude of change reported after sinus surgery.

The clinical meaningfulness of the benefit observed with OPN-375 in this study is also addressed by direct measurement of subject-perceived change in overall disease burden (PGIC). After 24 weeks of treatment, approximately 80% of subjects treated with OPN-375 reported improvement, suggesting that benefit is not confined to a minority of treated subjects, and 44% and 54% (low and high dose groups, respectively) reported much or very much improvement (vs 26% of the placebo group), supporting the conclusion that the magnitude of benefit with OPN-375 treatment is perceived as important by patients with the disease. Together, outcomes on patient-reported measures supporting clinically meaningful benefits of OPN-375 treatment, including SNOT-22 and PGIC, suggest that the benefits demonstrated on the co-primary measures of symptoms and CT scans are recognizable to subjects, and support the conclusion that improvement with OPN-375 treatment is clinically (not just statistically) meaningful.

Composite symptom score is the first co-primary endpoint and establishes the clinical effect of treatment. For CSS, subjects with NP and subjects without NP had similar magnitude of effect to the total FAS population. In addition, all subgroup active-vs-placebo treatment differences reached nominal statistical significance in both OPN-375 dose groups. Data showing a similar symptom benefit in the subgroup of chronic sinusitis subjects without NP are particularly important because they indicate that the symptom improvement with OPN-375 cannot be attributed solely to the treatment of polyps in the nasal cavity.

Opacified volume was the second co-primary endpoint and was intended to establish that the anatomic site of action included the sinus cavities and not simply the nasal cavity. Overall, the change from baseline produced by OPN-375 in CS subgroups with and without nasal polyps was similar in magnitude, suggesting similarity of biological response. However, entirely independent of the effects of OPN-375, in this trial, substantial differences in placebo response between phenotypic subgroups were observed. Further, numerical effects appeared sensitive to management of intercurrent events, likely attributable to the large effect of a small number of outliers on mean values. As a result, for the non-polyp sub-population, the primary estimand produced somewhat different results from the primary estimand and SE-2, resolving the large differences in placebo response compared to other subgroups (and compared to Study B) and finding the high dose group to have a similar placebo-subtracted change to both the FAS analysis and to the NP subgroup (though the low-dose group did not).

Treatment with OPN-375 was well tolerated and showed no unusual or unexpected events in the context of expectations for other combination products delivering fluticasone and expectations for an intranasal topically acting corticosteroid.

In a population of chronic sinusitis patients with proven intra-sinus disease at baseline, the totality of clinically important results across multiple substantiating measures in Study A, including the composite symptom co-primary endpoint, quality of life outcomes, and other measures, and most notably including a large reduction in risk of acute exacerbations, collectively support the benefit of OPN-375 treatment of CS. This benefit cannot be attributed solely to treatment of nasal polyps, and the CT-scan co-primary endpoint provides direct evidence of a treatment effect inside the sinus cavities.

### Example 4: Comparison of 3205 and 3206 Studies

Presented here are results using pooled data from Studies A and B for selected measures and the various populations.

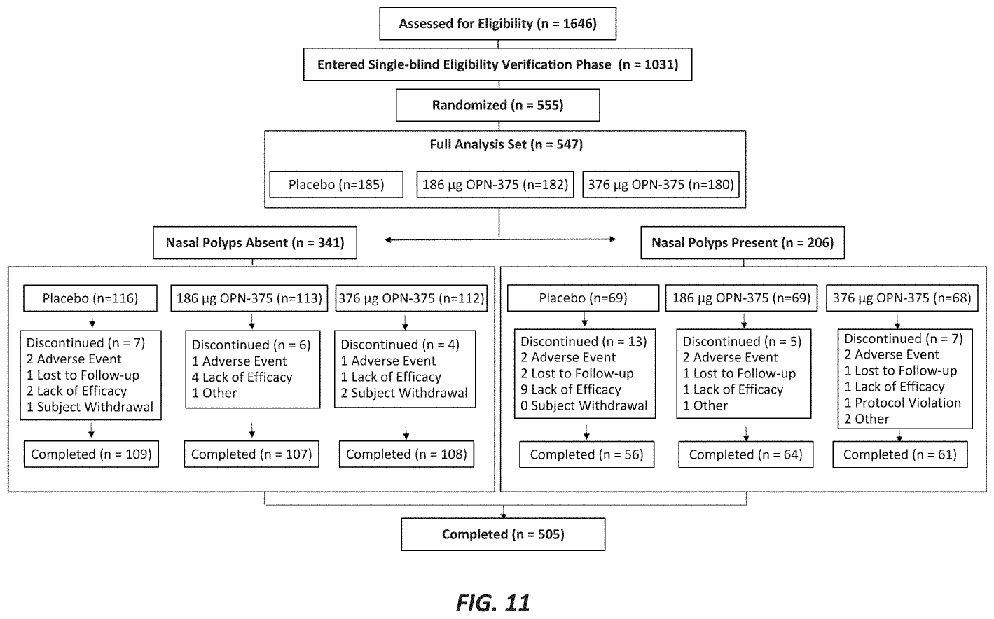

Overall, 1646 subjects were screened for eligibility for inclusion in the 2 pivotal studies in CS. Of the 555 subjects randomized between Studies A and B, 547 subjects received study drug and constituted the FAS of pooled data used for integrated analyses, with 341 and 206 subjects in each of the NP absent and present phenotypes, respectively (FIG. 11). Most subjects (92.3%) completed the study, with few subjects discontinuing due to lack of efficacy (18, 3.3%), adverse events (11, 2.0%), lost to follow-up (5, 0.9%), subject withdrawal (3, 0.5%), protocol violations (1, 0.2%), or other reasons (4, 0.7%).

Demographic characteristics were generally similar across treatment groups. Most subjects were White and not Hispanic or Latino (Table 23), approximately 15% were 65 years of age, and 40-50% were female. History of prior sinus surgery was similar across randomized groups, with approximately half of NP subjects reporting prior surgery and approximately ⅓ of non-NP subjects reporting prior surgery (Table 24).

TABLE 23
Summary of Subject Demographics by Nasal Polyp Status at Baseline.

| | CS Patients With Nasal Polyps | | | | CS Patients Without Nasal Polyps | | | |
|---|---|---|---|---|---|---|---|---|
| | | OPN-375 | OPN-375 | All | | OPN-375 | OPN-375 | All |
| | Placebo | 186 μg | 372 μg | Active | Placebo | 186 μg | 372 μg | Active |
| Parameter | (N = 69) | (N = 69) | (N = 68) | (N = 137) | (N = 116) | (N = 113) | (N = 112) | (N = 225) |
| Age (years) | | | | | | | | |
| Mean | 47.6 | 48.7 | 48.9 | 48.8 | 49.9 | 47.2 | 50.0 | 48.6 |
| SD | 15.17 | 13.75 | 13.44 | 13.55 | 13.60 | 13.64 | 13.49 | 13.61 |
| ≤65 | 57 (82.6) | 61 (88.4) | 58 (85.3) | 119 (86.8) | 97 (83.6) | 98 (86.7) | 95 (84.8) | 193 (85.8) |
| years, n | | | | | | | | |
| (%) | | | | | | | | |
| >65 | 12 (17.4) | 8 (11.6) | 10 (14.7) | 18 (13.2) | 19 (16.4) | 15 (13.3) | 17 (15.2) | 32 (14.2) |
| years, n | | | | | | | | |
| (%) | | | | | | | | |
| Gender, n (%) | | | | | | | | |
| Male | 42 (60.9) | 43 (62.3) | 40 (58.8) | 83 (60.6) | 60 (51.7) | 53 (46.9) | 60 (53.6) | 113 (50.2) |
| Female | 27 (39.1) | 26 (37.7) | 28 (41.2) | 54 (39.4) | 56 (48.3) | 60 (53.1) | 52 (46.4) | 112 (49.8) |
| Weight (kg) | | | | | | | | |
| Mean | 82.59 | 86.12 | 83.00 | 84.58 | 80.33 | 76.18 | 82.92 | 79.53 |
| SD | 19.873 | 22.227 | 18.983 | 20.675 | 19.515 | 15.902 | 19.779 | 18.212 |
| Race, n (%) | | | | | | | | |
| White | 62 (89.9) | 60 (87.0) | 61 (89.7) | 121 (88.3) | 111 (95.7) | 109 (96.5) | 108 (96.4) | 217 (96.4) |
| Black or | 4 (5.8) | 8 (11.6) | 4 (5.9) | 12 (8.8) | 3 (2.6) | 3 (2.7) | 0 | 3 (1.3) |
| African | | | | | | | | |
| American | | | | | | | | |
| American | 0 | 1 (1.4) | 0 | 1 (0.7) | 0 | 0 | 0 | 0 |
| Indian | | | | | | | | |
| or Alaska | | | | | | | | |
| Native | | | | | | | | |
| Asian | 2 (2.9) | 0 | 2 (2.9) | 2 (1.5) | 2 (1.7) | 1 (0.9) | 3 (2.7) | 4 (1.8) |
| Other | 1 (1.4) | 0 | 1 (1.5) | 1 (0.7) | 0 | 0 | 1 (0.9) | 1 (0.4) |
| Ethnicity, n (%) | | | | | | | | |
| Hispanic | 7 (10.1) | 1 (1.4) | 5 (7.4) | 6 (4.4) | 2 (1.7) | 2 (1.8) | 4 (3.6) | 6 (2.7) |
| or Latino | | | | | | | | |
| Not | 62 (89.9) | 68 (98.6) | 63 (92.6) | 131 (95.6) | 114 (98.3) | 111 (98.2) | 108 (96.4) | 219 (97.3) |
| Hispanic | | | | | | | | |
| or Latino | | | | | | | | |

Abbreviations:
N = number of subjects;
SD = standard deviation;
n (%) = number (percentage) of subjects in the subset.
Note:
Percentages are based on non-missing data

TABLE 24
Summary of Prior Sinus Surgery for Subjects by Polyp Status at Baseline.

| | With Nasal Polyps | | | | Without Nasal Polyps | | | |
|---|---|---|---|---|---|---|---|---|
| | | OPN-375 | OPN-375 | All | | OPN-375 | OPN-375 | All |
| | PBO | 186 μg | 372 μg | Active | PBO | 186 μg | 372 μg | Active |
| Parameter | (N = 69) | (N = 69) | (N = 68) | (N = 137) | (N = 116) | (N = 113) | (N = 112) | (N = 225) |

Prior Sinus Surgery (only ethmoidectomy or maxillary antrostomy)

| | | | | | | | | |
|---|---|---|---|---|---|---|---|---|
| Yes, n (%) | 32 (46.4) | 37 (53.6) | 31 (45.6) | 68 (49.6) | 39 (33.6) | 39 (34.5) | 37 (33.0) | 76 (33.8) |
| No, n (%) | 37 (53.6) | 32 (46.4) | 37 (54.4) | 69 (50.4) | 77 (66.4) | 74 (65.5) | 75 (67.0) | 149 (66.2) |

Abbreviations:
N = number of subjects;
PBO = placebo;
SE = standard error;
SD = standard deviation;
n (%) = number (percentage) of subjects in the subset.

Computation of Efficacy Results

The efficacy endpoints presented using pooled data analyses of the 2 pivotal studies by population and subgroup are summarized below (Table 25).

TABLE 25
Populations Analyzed by Endpoint.

| | | FAS¹ | | | |
|---|---|---|---|---|---|
| | | Overall | CRSsNP² | CRSwNP³ | Sub- |
| Endpoint | Outcome Measure | (Pooled) | Phenotype | Phenotype | groups⁴ |
| Co- | Change from | √ | √ | √ | √ |
| primary | baseline to the end | | | | |
| | of Week 4 in | | | | |
| | average total | | | | |
| | instantaneous AM | | | | |
| | CSS consisting of: | | | | |
| | nasal congestion, | | | | |
| | nasal discharge | | | | |
| | (anterior and/or | | | | |
| | posterior), and | | | | |
| | facial pain/ | | | | |
| | pressure sensation | | | | |
| | (overall | | | | |
| | population, | | | | |
| | subgroup | | | | |
| | populations by | | | | |
| | polyp status) | | | | |
| | Change from | √ | √ | √ | √ |
| | baseline to Week | | | | |
| | 24/ET in the | | | | |
| | APOV in the | | | | |
| | ethmoid and | | | | |
| | maxillary sinuses | | | | |
| Key | Frequency | √ | √ | √ | — |
| Secondary | of acute | | | | |
| | exacerbations | | | | |
| | of CRS | | | | |
| | Composite | √ | √ | √ | — |
| | Symptom Score | | | | |
| | (Change from | | | | |
| | baseline to Week | | | | |
| | 4) in subjects | | | | |
| | entering trial | | | | |
| | using a standard- | | | | |
| | delivery nasal | | | | |
| | steroid for | | | | |
| | treatment of CS | | | | |
| Other | SNOT-22 | √ | √ | √ | — |
| Secondary | (Change from | | | | |
| | baseline to Week | | | | |
| | 24/ET) in subjects | | | | |
| | entering trial | | | | |
| | using a standard- | | | | |
| | delivery nasal | | | | |
| | steroid for | | | | |
| | treatment of CS | | | | |
| | Global PSQI | √ | √ | √ | — |
| | Score (Change | | | | |
| | from baseline to | | | | |
| | Week 24/ET) | | | | |
| | CSS change over | √ | √ | √ | — |
| | time (baseline to | | | | |
| | Weeks 8, 12) | | | | |
| | Individual | √ | √ | √ | — |
| | cardinal symptom | | | | |
| | scores (Change | | | | |
| | from baseline to | | | | |
| | Weeks 4, 8, 12) | | | | |
| | Time to first acute | √ | √ | √ | — |
| | exacerbation of | | | | |
| | CRS through | | | | |
| | Week 24/ET | | | | |
| | Percent of | √ | √ | √ | — |
| | subjects with | | | | |
| | APOV | | | | |
| | improvement ≥ | | | | |
| | mean | | | | |
| | improvement in | | | | |
| | patients who | | | | |
| | reported being | | | | |
| | clinically | | | | |
| | improved by | | | | |
| | PGIC at Week 4 | | | | |
| | and 24/ET | | | | |

Abbreviations:
AM = morning;
APOV = average of the percentages of opacified volume;
CRS = chronic rhinosinusitis;
CRSsNP = chronic rhinosinusitis without nasal polyps;
CRSwNP = chronic rhinosinusitis with nasal polyps;
CS = chronic sinusitis;
CSS = composite symptom score;
DNS = daily nasal symptoms;
FAS = Full Analysis Set;
ET = early termination;
PSQI = Pittsburgh Sleep Quality Index;
SNOT-22 = Sinonasal Outcome Test-22 item
¹Full analysis set is pooled data for all subjects from Studies A and B irrespective of the presence of nasal polyps
²Subjects without nasal polyps from Studies A and B
³Subjects with nasal polyps from Studies A and B. A single incorrectly randomized subject (081410) from Study B is included in this pool.
⁴Subgroups included age, gender, race, and region

Composite Symptom Score

Change from baseline through Week 4 in combined symptom score (instantaneous, AM) using pooled data from Studies A and B is presented in Table 26. Baseline mean CSS scores were similar across randomized groups and reflected moderate disease. Placebo-subtracted statistically significant reductions from baseline were found for active treatment, with similar magnitudes of change for both the OPN-375 186 μg and OPN-375 372 μg BID treatment groups. Treatment differences were robustly in favor of OPN-375, with similar magnitude improvement and statistical significance when the pre-specified primary estimand, the alternative primary estimand and SE-2 were applied to the data set.

TABLE 26
Composite Symptom Score (Change from Baseline to End of Week 4,
Instantaneous AM).

| | | | Week 4 | | | | |
|---|---|---|---|---|---|---|---|
| | Baseline | | | LSM CFB | | | |
| Treatment (BID) | n | Mean (SD) | n | (SE) | LSMD | 95% CI | P-value |

Primary Estimand

| | | | | | | | |
|---|---|---|---|---|---|---|---|
| All OPN-375 | 362 | 5.64 (1.720) | 357 | −1.62 (0.095) | −0.92 | −1.22, −0.62 | <0.001 |
| OPN-375 372 μg | 180 | 5.67 (1.750) | 177 | −1.67 (0.131) | −0.96 | −1.31, −0.62 | <0.001 |
| OPN-375 186 μg | 182 | 5.60 (1.695) | 180 | −1.58 (0.130) | −0.87 | −1.22, −0.53 | <0.001 |
| Placebo | 185 | 5.92 (1.778) | 180 | −0.70 (0.129) | — | — | — |

Alternative primary estimand - Intercurrent Events Include Antibiotics for AECRS

| | | | | | | | |
|---|---|---|---|---|---|---|---|
| All OPN-375 | 362 | 5.64 (1.720) | 357 | −1.59 (0.101) | −1.09 | −1.41, −0.77 | <0.001 |
| OPN-375 372 μg | 180 | 5.67 (1.750) | 177 | −1.64 (0.139) | −1.13 | −1.50, −0.76 | <0.001 |
| OPN-375 186 μg | 182 | 5.60 (1.695) | 180 | −1.55 (0.138) | −1.04 | −1.41, −0.67 | <0.001 |
| Placebo | 185 | 5.92 (1.778) | 180 | −0.50 (0.137) | — | — | — |

Supplementary Estimand 2

| | | | | | | | |
|---|---|---|---|---|---|---|---|
| All OPN-375 | 362 | 5.64 (1.720) | 358 | −1.65 (0.093) | −0.90 | −1.19, −0.61 | <0.001 |
| OPN-375 372 μg | 180 | 5.67 (1.750) | 177 | −1.70 (0.127) | −0.95 | −1.28, −0.61 | <0.001 |
| OPN-375 186 μg | 182 | 5.60 (1.695) | 181 | −1.60 (0.126) | −0.85 | −1.18, −0.51 | <0.001 |
| Placebo | 185 | 5.92 (1.778) | 181 | −0.75 (0.125) | — | — | — |

Abbreviations:
AECRS = acute exacerbations of chronic rhinosinusitis;
AM = morning;
BID = twice daily;
CI = confidence interval;
CFB = change from baseline;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (2, 4), treatment-by-week interaction, and continuous covariate: baseline 7-day average score, with baseline score-by-week interaction.

As in the overall study population, composite symptom scores improved significantly in the pooled population of CS patients without polyps visible in the nasal cavity at baseline (Table 27). Baseline symptom scores were comparable across randomization groups in both subpopulations. Statistically significant reductions versus placebo were observed with OPN-375 treatment, with a similar magnitude of improvement in both the OPN-375 186 μg and OPN-375 372 μg BID treatment groups. The finding of significant symptom improvement with OPN-375 in CS patients without nasal polyps was robust to analytic approach, with improvement in symptoms under the primary estimand, the alternative primary estimand, and SE-2.

TABLE 27
Composite Symptom Score by Phenotype (Change from Baseline to End of
Week 4).

| | | | Week 4 | | | | |
|---|---|---|---|---|---|---|---|
| Population | Baseline | | | LSM (SE) | | | |
| Treatment (BID) | n | Mean (SD) | n | CFB | LSMD | 95% CI | P-value |

Primary Estimand

| | | | | | | | |
|---|---|---|---|---|---|---|---|
| CS Patients With Nasal Polyps | | | | | | | |
| All OPN-375 | 137 | 5.32 (1.802) | 135 | −1.74 (0.163) | −1.08 | −1.57, −0.59 | <0.001 |
| OPN-375 372 μg | 68 | 5.26 (1.800) | 67 | −1.78 (0.218) | −1.12 | −1.69, −0.55 | <0.001 |
| OPN-375 186 μg | 69 | 5.37 (1.814) | 68 | −1.70 (0.218) | −1.04 | −1.61, −0.47 | <0.001 |
| Placebo | 69 | 5.81 (1.841) | 68 | −0.66 (0.217) | — | — | — |
| CS Patients Without Nasal Polyps | | | | | | | |
| All OPN-375 | 225 | 5.83 (1.643) | 222 | −1.53 (0.117) | −0.81 | −1.19, −0.43 | <0.001 |
| OPN-375 372 μg | 112 | 5.92 (1.678) | 110 | −1.58 (0.163) | −0.86 | −1.30, −0.42 | <0.001 |
| OPN-375 186 μg | 113 | 5.74 (1.610) | 112 | −1.48 (0.162) | −0.76 | −1.20, −0.32 | <0.001 |
| Placebo | 116 | 5.99 (1.744) | 112 | −0.72 (0.161) | — | — | — |

Alternative primary estimand - Intercurrent Events Include Antibiotics for AECRS

| | | | | | | | |
|---|---|---|---|---|---|---|---|
| CS Patients With Nasal Polyps | | | | | | | |
| All OPN-375 | 137 | 5.32 (1.802) | 135 | −1.73 (0.173) | −1.26 | −1.78, −0.74 | <0.001 |
| OPN-375 372 μg | 68 | 5.26 (1.800) | 67 | −1.80 (0.231) | −1.34 | −1.94, −0.73 | <0.001 |
| OPN-375 186 μg | 69 | 5.37 (1.814) | 68 | −1.65 (0.231) | −1.19 | −1.79, −0.58 | <0.001 |
| Placebo | 69 | 5.81 (1.841) | 68 | 0.46 (0.230) | — | — | — |
| CS Patients Without Nasal Polyps | | | | | | | |
| All OPN-375 | 225 | 5.83 (1.643) | 222 | −1.49 (0.124) | −0.98 | −1.38, −0.57 | <0.001 |
| OPN-375 372 μg | 112 | 5.92 (1.678) | 110 | −1.52 (0.173) | −1.01 | −1.47, −0.54 | <0.001 |
| OPN-375 186 μg | 113 | 5.74 (1.610) | 112 | −1.46 (0.172) | −0.95 | −1.42, −0.48 | <0.001 |
| Placebo | 116 | 5.99 (1.744) | 112 | −0.52 (0.171) | — | — | — |

Supplementary Estimand 2

| | | | | | | | |
|---|---|---|---|---|---|---|---|
| CS Patients With Nasal Polyps | | | | | | | |
| All OPN-375 | 137 | 5.32 (1.802) | 135 | −1.74 (0.160) | −0.97 | −1.45, −0.50 | <0.001 |
| OPN-375 372 μg | 68 | 5.26 (1.800) | 67 | −1.78 (0.213) | −1.01 | −1.56, −0.46 | <0.001 |
| OPN-375 186 μg | 69 | 5.37 (1.814) | 68 | −1.70 (0.212) | −0.93 | −1.48, −0.38 | <0.001 |
| Placebo | 69 | 5.81 (1.841) | 67 | −0.77 (0.212) | — | — | — |
| CS Patients Without Nasal Polyps | | | | | | | |
| All OPN-375 | 225 | 5.83 (1.643) | 223 | −1.57 (0.113) | −0.84 | −1.21, −0.47 | <0.001 |
| OPN-375 372 μg | 112 | 5.92 (1.678) | 110 | −1.63 (0.158) | −0.90 | −1.33, −0.47 | <0.001 |
| OPN-375 186 μg | 113 | 5.74 (1.610) | 113 | −1.51 (0.156) | −0.78 | −1.21, −0.36 | <0.001 |
| Placebo | 116 | 5.99 (1.744) | 114 | −0.73 (0.155) | — | — | — |

Abbreviations:
AECRS = acute exacerbations of chronic rhinosinusitis;
BID = twice daily;
CFB = change from baseline;
CI = confidence interval;
CS = chronic sinusitis;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (2, 4), treatment-by-week interaction, and continuous covariate: baseline 7-day average score, with baseline score-by-week interaction, treatment-by-subgroup, subgroup-by-week, and treatment-by-subgroup-by-week interaction.

Average of the Percentages of Opacified Volume in the Ethmoid and Maxillary Sinus Cavities (APOV)

Change from baseline through Week 24 in APOV using pooled data from Studies A and B is presented in Table 28. The purpose of this measure at baseline was to assure the presence of intra-sinus disease, and the purpose of measuring change over time was to determine if treatment effects were occurring in the sinus cavities and not only in the nasal cavity, particularly because OPN-375 is a nasally administered topically acting product. At baseline, mean ethmoid and maxillary sinus volume opacification was ˜65% and similar across the 3 randomized treatment groups (Table 28). Following 24 weeks of treatment, mean APOV improved (decreased) with OPN-375 but not the placebo comparator. The improvement in sinus opacification with OPN-375 was robust to analytic approach, showing clear statistical significance under the primary estimand, the alternative primary estimand, and SE-2.

TABLE 28
Average of the Percentages of Opacified Volume in Ethmoid and Maxillary
Sinuses (Change from Baseline to Week 24/ET)

| | | | Week 24/ET | | | | |
|---|---|---|---|---|---|---|---|
| | Baseline | | | LSM (SE) | | | |
| Treatment (BID) | n | Mean (SD) | n | CFB | LSMD | 95% CI | P-value |

Primary Estimand

| | | | | | | | |
|---|---|---|---|---|---|---|---|
| All OPN-375 | 362 | 65.75 (19.392) | 338 | −5.54 (0.840) | −5.48 | −8.05, −2.90 | <0.001 |
| OPN-375 372 μg | 180 | 65.92 (18.991) | 170 | −5.35 (1.142) | −5.29 | −8.25, −2.32 | <0.001 |
| OPN-375 186 μg | 182 | 65.57 (19.832) | 168 | −5.73 (1.141) | −5.67 | −8.67, −2.66 | <0.001 |
| Placebo | 185 | 66.97 (19.420) | 165 | −0.06 (1.130) | — | — | — |

Alternative primary estimand—Intercurrent Events Include Antibiotics for AECRS

| | | | | | | | |
|---|---|---|---|---|---|---|---|
| All OPN-375 | 362 | 65.75 (19.392) | 341 | −3.68 (0.898) | −6.20 | −8.92, −3.47 | <0.001 |
| OPN-375 372 μg | 180 | 65.92 (18.991) | 170 | −3.72 (1.211) | −6.23 | −9.39, −3.08 | <0.001 |
| OPN-375 186 μg | 182 | 65.57 (19.832) | 171 | −3.65 (1.211) | −6.16 | −9.31, −3.01 | <0.001 |
| Placebo | 185 | 66.97 (19.420) | 167 | 2.51 (1.197) | — | — | — |

Supplementary Estimand 2

| | | | | | | | |
|---|---|---|---|---|---|---|---|
| All OPN-375 | 362 | 65.75 (19.392) | 347 | −5.90 (0.849) | −5.45 | −8.03, −2.86 | <0.001 |
| OPN-375 372 μg | 180 | 65.92 (18.991) | 175 | −5.44 (1.150) | −4.99 | −7.96, −2.01 | 0.001 |
| OPN-375 186 μg | 182 | 65.57 (19.832) | 172 | −6.36 (1.143) | −5.91 | −8.92, −2.89 | <0.001 |
| Placebo | 185 | 66.97 (19.420) | 171 | −0.45 (1.137) | — | — | — |

Abbreviations:
AECRS = acute exacerbations of chronic rhinosinusitis;
ANCOVA = analysis of covariance;
BID = twice daily;
CFB = change from baseline;
CFB = change from baseline;
CI = confidence interval;
ET = early termination;
LSM = least squares mean;
LSMD = least squares mean difference (active − placebo);
SD = standard deviation;
SE = standard error
Note:
Inferential statistics are based on multiple imputation of a linear ANCOVA model including categorical effects for protocol, nasal polyp status, previous sinus surgery, treatment, and continuous baseline value as covariate.

Compared to CS patients with nasal polyps, CS patients entering Studies A and B without nasal polyps had slightly less sinus opacification at baseline (˜10% less), and correspondingly slightly smaller absolute numerical change scores than in response to treatment with OPN-375, with treatment producing nearly the same proportional treatment effect for patients either with or without nasal polyps. The improvement in APOV produced by OPN-375 over placebo for CS patients without nasal polyps was robustly statistically significant under each estimand (primary, alternative primary, and SE-2) and was similar in magnitude for each separate dose of OPN-375 (Table 29).

TABLE 29
Average of the Percentages of Opacified Volume in Ethmoid and Maxillary
Sinuses by Phenotype (Change from Baseline to Week 24/ET)

| | | | Week 24/ET | | | | |
|---|---|---|---|---|---|---|---|
| Population | Baseline | | | LSM (SE) | | | |
| Treatment (BID) | n | Mean (SD) | n | CFB | LSMD | 95% CI | P-value |

Primary Estimand
CS Patients With Nasal Polyps

| | | | | | | | |
|---|---|---|---|---|---|---|---|
| All OPN-375 | 137 | 73.00 (17.61) | 126 | −4.77 (1.508) | −6.69 | −10.88, −2.49 | 0.002 |
| OPN-375 372 μg | 68 | 73.60 (16.95) | 63 | −4.02 (1.963) | 5.93 | −10.75, −1.12 | 0.016 |
| OPN-375 186 μg | 69 | 72.41 (18.34) | 63 | −5.53 (1.961) | −7.44 | −12.35, −2.53 | 0.003 |
| Placebo | 69 | 73.10 (21.10) | 62 | 1.91 (1.950) | — | — | — |

CS Patients Without Nasal Polyps

| | | | | | | | |
|---|---|---|---|---|---|---|---|
| All OPN-375 | 225 | 61.33 (19.13) | 212 | −6.31 (1.048) | −4.76 | −8.03, −1.48 | 0.004 |
| OPN-375 372 μg | 112 | 61.26 (18.71) | 107 | −6.50 (1.412) | −4.95 | −8.70, −1.19 | 0.010 |
| OPN-375 186 μg | 113 | 61.40 (19.62) | 105 | −6.12 (1.447) | −4.57 | −8.39, −0.75 | 0.019 |
| Placebo | 116 | 63.32 (17.44) | 103 | −1.55 (1.413) | — | — | — |

Alternative primary estimand—Intercurrent Events Include Antibiotics for AECRS
CS Patients With Nasal Polyps

| | | | | | | | |
|---|---|---|---|---|---|---|---|
| All OPN-375 | 137 | 73.00 (17.61) | 128 | −4.18 (1.596) | −7.11 | −11.57, −2.66 | 0.002 |
| OPN-375 372 μg | 68 | 73.60 (16.95) | 63 | −3.89 (2.087) | −6.82 | −12.00, −1.64 | 0.010 |
| OPN-375 186 μg | 69 | 72.41 (18.34) | 65 | −4.48 (2.047) | −7.40 | −12.51, −2.29 | 0.005 |
| Placebo | 69 | 73.10 (21.10) | 62 | 2.93 (2.059) | — | — | — |

CS Patients Without Nasal Polyps

| | | | | | | | |
|---|---|---|---|---|---|---|---|
| All OPN-375 | 225 | 61.33 (19.13) | 213 | −3.18 (1.087) | −5.67 | −9.13, −2.21 | 0.001 |
| OPN-375 372 μg | 112 | 61.26 (18.71) | 107 | −3.39 (1.496) | −5.88 | −9.89, −1.87 | 0.004 |
| OPN-375 186 μg | 113 | 61.40 (19.62) | 106 | −2.97 (1.506) | −5.46 | −9.47, −1.44 | 0.008 |
| Placebo | 116 | 63.32 (17.44) | 105 | 2.49 (1.492) | — | — | — |

Supplementary Estimand—2
CS Patients With Nasal Polyps

| | | | | | | | |
|---|---|---|---|---|---|---|---|
| All OPN-375 | 137 | 73.00 (17.61) | 130 | −4.86 (1.510) | −5.64 | −9.91, −1.37 | 0.010 |
| OPN-375 372 μg | 68 | 73.60 (16.95) | 66 | −3.72 (1.962) | −4.50 | −9.41, 0.40 | 0.072 |
| OPN-375 186 μg | 69 | 72.41 (18.34) | 64 | −6.00 (1.973) | −6.78 | −11.74, −1.81 | 0.008 |
| Placebo | 69 | 73.10 (21.10) | 63 | 0.78 (1.970) | — | — | — |

CS Patients Without Nasal Polyps

| | | | | | | | |
|---|---|---|---|---|---|---|---|
| All OPN-375 | 225 | 61.33 (19.13) | 217 | −7.02 (1.030) | −5.35 | −8.62, −2.08 | 0.001 |
| OPN-375 372 μg | 112 | 61.26 (18.71) | 109 | −7.02 (1.422) | −5.35 | −9.14, −1.56 | 0.006 |
| OPN-375 186 μg | 113 | 61.40 (19.62) | 108 | −7.02 (1.414) | −5.35 | −9.15, −1.56 | 0.006 |
| Placebo | 116 | 63.32 (17.44) | 108 | −1.67 (1.395) | — | — | — |

Abbreviations:
AECRS = acute exacerbations of chronic rhinosinusitis;
BID = twice daily;
CFB = change from baseline;
CI = confidence interval;
CS = chronic sinusitis;
CSR = clinical study report;
LSM = least squares mean;
LSMD = least squares mean difference (active − placebo);
SD = standard deviation;
SE = standard error

Key Secondary Endpoints

Acute exacerbations were defined as an episode in which the patient experienced worsening of one or more of the core symptoms of CS (congestion, discharge, facial pain/pressure, loss of sense of smell) for a minimum of 3 days that also resulted in escalation of care (e.g., prescribing of an antibiotic or systemic steroid, acute care visit, ER visit). Pre-planned and Type 1 error-controlled analysis of the frequency of acute sinus exacerbations over the 24-week treatment period compared OPN-375 dose groups and placebo in pooled data from Studies A and B and used a negative binomial regression (NB2) model. The NB2 model used family-wise Type 1 error control and included the logarithm of exposure time as an offset variable.

In total, there were 76 exacerbations, of which 71 resulted in the use of antibiotics and 13 resulted in the use of oral steroids. Treatment with OPN-375 produced a large magnitude and statistically significant reduction in the incidence of disease exacerbations (Table 30). Specifically, exacerbations for all patients receiving OPN-375 were reduced by 61% compared to exacerbations among patients receiving placebo, with statistically significant reductions of 56% and 66% for the low and high doses, respectively.

TABLE 30
Frequency of Acute CRS Exacerbations over 24 Weeks.

| | | | LSM (SE) | IRR | |
|---|---|---|---|---|---|
| Treatment | | | Events/Patient | (Active/Placebo) | |
| Group (BID) | n | Events | (24 wks) | (95% CI) | P-value |
| All OPN-375 | 362 | 35 | 0.081 (0.0177) | 0.389 (0.226, 0.669) | 0.001 |
| OPN-375 | 180 | 15 | 0.072 (0.0218) | 0.343 (0.173, 0.680) | 0.002 |
| 372 μg | | | | | |
| OPN-375 | 182 | 20 | 0.092 (0.0255) | 0.441 (0.233, 0.832) | 0.012 |
| 186 μg | | | | | |
| Placebo | 185 | 41 | 0.208 (0.0446) | — | — |

Abbreviations:
BID = twice daily;
CI = confidence interval;
CRS = chronic rhinosinusitis;
IRR = incidence rate ratio;
LSM = least squares mean;
SE = standard error;
wks = weeks
Note:
Inferential statistics are based on a negative binomial regression (NB2) model including categorical effects for protocol, nasal polyp status, previous sinus surgery, treatment, and natural logarithm of treatment duration (days) as an offset parameter.

In the subpopulation of CS patients who did not have polyps in the nasal cavity at baseline, treatment with OPN-375 produced a reduction in the rate of AECRS compared to placebo of a roughly similar magnitude (˜53%; P=0.032), as observed in the overall population. There was a similar reduction in events in both dose groups (Table 31).

TABLE 31
Frequency of Acute CRS Exacerbations by Phenotype.

| | | | | | |
|---|---|---|---|---|---|
| Population | | | LSM (SE) | IRR | |
| Treatment | | | Events/Patient | (Active/Placebo) | |
| Group (BID) | n | Events | (24 wks) | (95% CI) | P-value |

CS Patients With Nasal Polyps

| | | | | | |
|---|---|---|---|---|---|
| All OPN-375 | 137 | 12 | 0.052 (0.0197) | 0.276 (0.112, 0.679) | 0.005 |
| OPN-375 | 68 | 4 | 0.038 (0.0215) | 0.203 (0.060, 0.685) | 0.010 |
| 372 μg | | | | | |
| OPN-375 | 69 | 8 | 0.070 (0.0311) | 0.376 (0.138, 1.022) | 0.055 |
| 186 μg | | | | | |
| Placebo | 69 | 17 | 0.187 (0.0673) | — | — |

CS Patients Without Nasal Polyps

| | | | | | |
|---|---|---|---|---|---|
| All OPN-375 | 225 | 23 | 0.113 (0.0270) | 0.472 (0.237, 0.939) | 0.032 |
| OPN-375 | 112 | 11 | 0.113 (0.0386) | 0.470 (0.204, 1.086) | 0.077 |
| 372 μg | | | | | |
| OPN-375 | 113 | 12 | 0.113 (0.0376) | 0.474 (0.208, 1.081) | 0.076 |
| 186 μg | | | | | |
| Placebo | 116 | 24 | 0.239 (0.0619) | — | — |

Abbreviations:
BID = twice daily;
CI = confidence interval;
CRS = chronic rhinosinusitis;
CS = chronic sinusitis;
IRR = incidence rate ratio;
LSM = least squares mean;
SE = standard error;
wks = weeks
Note:
Inferential statistics are based on a negative binomial regression (NB2) model including categorical effects for protocol, nasal polyp status, previous sinus surgery, treatment, and natural logarithm of treatment duration (days) as an offset parameter, with treatment-by-subgroup interaction.

CSS change from baseline through Week 4 in the subgroup of subjects entering the trial already using a standard-delivery nasal steroid for treatment of CS was analyzed using the same MMRM model used for the coprimary endpoint of CSS.

Baseline scores were similar across treatment groups. Both doses of OPN-375 produced significant improvement compared to placebo in this population, a finding that was robust to analytic approach (primary, alternative primary, and SE-2 estimands), with a magnitude similar to the symptom improvement observed in the overall population, particularly using the alternative primary estimand (Table 32). This suggests that there is a treatment effect of OPN-375 in CS patients entering these trials, independent of whether or not patients were symptomatic while using standard-delivery nasal steroids.

TABLE 32
Composite Symptom Scores for Patients Using Standard-Delivery Nasal
Steroid at Trial Entry (Change from Baseline to End of Week 4).

| | | | Week 4 | | | | |
|---|---|---|---|---|---|---|---|
| | Baseline | | | LSM (SE) | | | |
| Treatment (BID) | n | Mean (SD) | n | CFB | LSMD | 95% CI | P-value |

Primary Estimand

| | | | | | | | |
|---|---|---|---|---|---|---|---|
| AII OPN-375 | 172 | 5.63 (1.744) | 169 | −1.46 (0.132) | −0.70 | −1.09, −0.30 | <0.001 |
| OPN-375 372 μg | 85 | 5.54 (1.749) | 83 | −1.40 (0.184) | −0.63 | −1.10, −0.16 | 0.009 |
| OPN-375 186 μg | 87 | 5.71 (1.746) | 86 | −1.53 (0.180) | −0.76 | −1.23, −0.30 | 0.001 |
| Placebo | 108 | 5.84 (1.730) | 104 | −0.77 (0.163) | — | — | — |

Alternative primary estimand—Intercurrent Events Include Antibiotics for AECRS

| | | | | | | | |
|---|---|---|---|---|---|---|---|
| All OPN-375 | 172 | 5.63 (1.744) | 169 | −1.47 (0.144) | −1.03 | −1.46, −0.60 | <0.001 |
| OPN-375 372 μg | 85 | 5.54 (1.749) | 83 | −1.39 (0.201) | −0.95 | −1.46, −0.44 | <0.001 |
| OPN-375 186 μg | 87 | 5.71 (1.746) | 86 | −1.55 (0.196) | −1.11 | −1.62, −0.60 | <0.001 |
| Placebo | 108 | 5.84 (1.730) | 104 | −0.44 (0.178) | — | — | — |

Supplementary Estimand 2

| | | | | | | | |
|---|---|---|---|---|---|---|---|
| All OPN-375 | 172 | 5.63 (1.744) | 169 | −1.50 (0.128) | −0.66 | −1.04, −0.28 | <0.001 |
| OPN-375 372 μg | 85 | 5.54 (1.749) | 83 | −1.46 (0.178) | −0.62 | −1.07, −0.17 | 0.007 |
| OPN-375 186 μg | 87 | 5.71 (1.746) | 86 | −1.53 (0.173) | −0.69 | −1.14, −0.25 | 0.002 |
| Placebo | 108 | 5.84 (1.730) | 105 | −0.84 (0.157) | — | — | — |

Abbreviations:
AECRS = acute exacerbations of chronic rhinosinusitis;
BID = twice daily;
CFB = change from baseline;
CI = confidence interval;
LSM = least squares mean;
LSMD = least squares
mean difference (active − placebo);
SD = standard deviation;
SE = standard error

OPN-375 produced improvement compared to placebo in CS patients who did not have polyps in the nasal cavity at baseline who entered the study using standard-delivery nasal steroids. The improvement over placebo was statistically significant for all OPN-375 patients vs placebo irrespective of analytic approach (primary, alternative primary, SE-2 estimand), with the largest treatment difference and with statistical significance for individual dose groups under the alternative primary estimand (Table 33).

TABLE 33
Composite Symptom Scores for Patients Using Standard-Delivery Nasal
Steroid at Trial Entry by Phenotype (Change from Baseline to End of Week 4).

| | | | Week 4 | | | | |
|---|---|---|---|---|---|---|---|
| | Baseline | | | LSM CFB | | | |
| Treatment (BID) | n | Mean (SD) | n | (SE) | LSMD | 95% CI | P-value |

Primary Estimand
CS Patients With Nasal Polyps

| | | | | | | | |
|---|---|---|---|---|---|---|---|
| All OPN-375 | 74 | 5.51 (1.923) | 72 | −1.57 (0.216) | −0.90 | −1.52, −0.29 | 0.004 |
| OPN-375 372 μg | 36 | 5.33 (1.875) | 35 | −1.52 (0.292) | −0.85 | −1.58, 0.12 | 0.023 |
| OPN-375 186 μg | 38 | 5.68 (1.977) | 37 | −1.63 (0.287) | −0.96 | −1.67, −0.25 | 0.009 |
| Placebo | 44 | 5.79 (1.718) | 44 | 0.67 (0.268) | — | — | — |

CS Patients Without Nasal Polyps

| | | | | | | | |
|---|---|---|---|---|---|---|---|
| All OPN-375 | 98 | 5.71 (1.601) | 97 | −1.36 (0.168) | −0.54 | −1.06, −0.01 | 0.045 |
| OPN-375 372 μg | 49 | 5.69 (1.652) | 48 | −1.28 (0.238) | −0.45 | −1.07, 0.16 | 0.150 |
| OPN-375 186 μg | 49 | 5.73 (1.564) | 49 | −1.45 (0.235) | −0.62 | −1.24, −0.00 | 0.050 |
| Placebo | 64 | 5.86 (1.751) | 60 | −0.83 (0.212) | — | — | — |

Alternative primary estimand—Intercurrent Events Include Antibiotics for AECRS
CS Patients With Nasal Polyps

| | | | | | | | |
|---|---|---|---|---|---|---|---|
| All OPN-375 | 74 | 5.51 (1.923) | 72 | −1.62 (0.234) | −1.23 | −1.90, −0.56 | <0.001 |
| OPN-375 372 μg | 36 | 5.33 (1.875) | 35 | −1.55 (0.317) | −1.16 | −1.96, −0.37 | 0.004 |
| OPN-375 186 μg | 38 | 5.68 (1.977) | 37 | −1.68 (0.312) | −1.30 | −2.08, −0.52 | 0.001 |
| Placebo | 44 | 5.79 (1.718) | 44 | −0.39 (0.291) | — | — | — |

CS Patients Without Nasal Polyps

| | | | | | | | |
|---|---|---|---|---|---|---|---|
| All OPN-375 | 98 | 5.71 (1.601) | 97 | −1.33 (0.183) | −0.88 | −1.45, −0.30 | 0.003 |
| OPN-375 372 μg | 49 | 5.69 (1.652) | 48 | −1.23 (0.259) | −0.77 | −1.44, −0.10 | 0.025 |
| OPN-375 186 μg | 49 | 5.73 (1.564) | 49 | −1.44 (0.256) | −0.98 | −1.65, −0.31 | 0.005 |
| Placebo | 64 | 5.86 (1.751) | 60 | −0.46 (0.231) | — | — | — |

Supplementary Estimand—2
CS Patients With Nasal Polyps

| | | | | | | | |
|---|---|---|---|---|---|---|---|
| All OPN-375 | 74 | 5.51 (1.923) | 72 | −1.59 (0.210) | −0.75 | −1.34, −0.15 | 0.014 |
| OPN-375 372 μg | 36 | 5.33 (1.875) | 35 | −1.53 (0.282) | −0.69 | −1.39, 0.01 | 0.054 |
| OPN-375 186 μg | 38 | 5.68 (1.977) | 37 | −1.64 (0.278) | −0.80 | −1.49, −0.11 | 0.023 |
| Placebo | 44 | 5.79 (1.718) | 43 | −0.84 (0.260) | — | — | — |

CS Patients Without Nasal Polyps

| | | | | | | | |
|---|---|---|---|---|---|---|---|
| All OPN-375 | 98 | 5.71 (1.601) | 97 | −1.41 (0.162) | −0.58 | −1.08, −0.08 | 0.023 |
| OPN-375 372 μg | 49 | 5.69 (1.652) | 48 | −1.38 (0.229) | −0.55 | −1.14, 0.04 | 0.068 |
| OPN-375 186 μg | 49 | 5.73 (1.564) | 49 | −1.44 (0.226) | −0.61 | −1.21, −0.02 | 0.042 |
| Placebo | 64 | 5.86 (1.751) | 62 | −0.83 (0.202) | — | — | — |

Abbreviations:
AECRS = acute exacerbations of chronic rhinosinusitis;
AM = morning;
BID = twice daily;
CFB = change from baseline;
CI = confidence interval;
CS = chronic sinusitis;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (2, 4), treatment-by-week interaction, and continuous covariate: baseline 7-day average score, with baseline score-by-week interaction; treatment-by-subgroup, subgroup-by-week, and treatment-by-subgroup-by-week interaction.

Other Secondary Endpoints

The SNOT-22 is a broad measure of the burden of illness, including symptoms, function, and other domains, and considered a disease-specific quality of life instrument. It is widely used for assessing response to both surgical and medical therapy in patients with CRS with or without NP. The magnitude of minimally clinically important changes in the SNOT-22 (8.9 points) and the expected treatment effect from surgery in large population samples have been previously published (Soler et al., 2018).

SNOT-22 total scores improved with OPN-375 compared to placebo (all, high, low dose groups). Under both the primary and alternative primary estimand, the magnitude of improvement was greater than the minimal clinically important difference, and the improvement over placebo was nominally statistically significant, for all OPN-375 and for each individual dose. (Table 34).

TABLE 34
Sinonasal Outcome Test-22 Total Score (Change from Baseline to Week 24/ET).

| | | | Week 24/ET | | | | |
|---|---|---|---|---|---|---|---|
| | Baseline | | | LSM (SE) | | | |
| Treatment (BID) | n | Mean (SD) | n | CFB | LSMD | 95% CI | P-value |

Primary Estimand

| | | | | | | | |
|---|---|---|---|---|---|---|---|
| All OPN-375 | 360 | 49.95 (18.70) | 332 | −18.46 (1.01) | −9.62 | −12.86, −6.38 | <0.001 |
| OPN-375 372 μg | 180 | 49.26 (19.38) | 164 | −19.03 (1.39) | −10.19 | −13.93, −6.44 | <0.001 |
| OPN-375 186 μg | 180 | 50.64 (18.02) | 168 | −17.90 (1.38) | −9.06 | −12.78, −5.33 | <0.001 |
| Placebo | 183 | 49.27 (19.55) | 163 | −8.84 (1.39) | — | — | — |

Alternative primary estimand—Intercurrent Events Include Antibiotics for AECRS

| | | | | | | | |
|---|---|---|---|---|---|---|---|
| All OPN-375 | 360 | 49.95 (18.70) | 334 | −15.73 (1.15) | −10.65 | −14.34, −6.96 | <0.001 |
| OPN-375 372 μg | 180 | 49.26 (19.38) | 165 | −16.72 (1.59) | −11.64 | −15.90, −7.37 | <0.001 |
| OPN-375 186 μg | 180 | 50.64 (18.02) | 169 | −14.75 (1.57) | −9.66 | −13.91, −5.42 | <0.001 |
| Placebo | 183 | 49.27 (19.55) | 166 | −5.08 (1.57) | — | — | — |

Abbreviations:
AECRS = acute exacerbations of chronic rhinosinusitis;
BID = twice daily;
CFB = change from baseline;
CFB = change from baseline;
CI = confidence interval;
ET = early termination;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (4, 8, 12, 24/ET), treatment-by-week interaction, and continuous covariate: baseline score, with baseline score-by-week interaction.
Source: ISE Ad hoc Table 2.5a and ISE Ad hoc Table 14.2.6.1

At baseline, the severity of disease measured by SNOT-22 was similar for CS subpopulations with and without NP in this trial. With both doses of OPN-375, the SNOT-22 total score improved from baseline to Week 24 by a magnitude greater than the minimum clinically important difference for the SNOT-22 scale for subpopulations of subjects either with or without polyps in the nasal cavity (Table 35). The treatment differences versus placebo in the LS mean change from baseline to Week 24 were statistically significant in favor of active treatment (low, high, and all doses) under both the primary and alternative primary estimands. Although the treatment effect was greater than the MCID in both subpopulations, the magnitude of effect measured by this scale was greater for the subpopulation with NP, potentially because this scale captured a treatment effect on both the sinuses and also on the nasal cavity polyps in that population.

TABLE 35
Sinonasal Outcome Test 22 Total Score by Phenotype (Change from Baseline to Week 24/ET).

| | | | Week 24/ET | | | | |
|---|---|---|---|---|---|---|---|
| | Baseline | | | LSM CFB | | | |
| Treatment (BID) | n | Mean (SD) | n | (SE) | LSMD | 95% CI | P-value |

Primary Estimand
CS Patients With Nasal Polyps

| | | | | | | | |
|---|---|---|---|---|---|---|---|
| All OPN-375 | 135 | 49.98 (18.86) | 121 | −19.54 (1.75) | −14.67 | −19.99, −9.35 | <0.001 |
| OPN-375 372 μg | 68 | 48.75 (19.96) | 59 | −21.50 (2.36) | −16.63 | −22.79, −10.46 | <0.001 |
| OPN-375 186 μg | 67 | 51.22 (17.72) | 62 | −17.59 (2.32) | −12.71 | −18.82, −6.61 | <0.001 |
| Placebo | 69 | 49.04 (21.36) | 58 | −4.87 (2.36) | — | — | — |

CS Patients Without Nasal Polyps

| | | | | | | | |
|---|---|---|---|---|---|---|---|
| All OPN-375 | 225 | 49.93 (18.64) | 211 | −18.39 (1.23) | −6.73 | −10.78, −2.68 | 0.001 |
| OPN-375 372 μg | 112 | 49.57 (19.10) | 105 | −18.17 (1.72) | −6.52 | −11.20, −1.84 | 0.006 |
| OPN-375 186 μg | 113 | 50.29 (18.26) | 106 | −18.61 (1.71) | −6.95 | −11.62, −2.28) | 0.004 |
| Placebo | 114 | 49.41 (18.46) | 105 | −11.66 (1.72) | — | — | — |

Alternative primary estimand—Intercurrent Events Include Antibiotics for AECRS
CS Patients With Nasal Polyps

| | | | | | | | |
|---|---|---|---|---|---|---|---|
| All OPN-375 | 135 | 49.98 (18.86) | 123 | −16.63 (1.97) | −14.94 | −20.98, −8.89 | <0.001 |
| OPN-375 372 μg | 68 | 48.75 (19.96) | 60 | −19.57 (2.67) | −17.88 | −24.89, −10.87 | <0.001 |
| OPN-375 186 μg | 67 | 51.22 (17.72) | 63 | −13.68 (2.63) | −11.99 | −18.94, −5.04 | <0.001 |
| Placebo | 69 | 49.04 (21.36) | 60 | −1.69 (1.66) | — | — | — |

CS Patients Without Nasal Polyps

| | | | | | | | |
|---|---|---|---|---|---|---|---|
| All OPN-375 | 225 | 49.93 (18.64) | 211 | −15.71 (1.41) | −8.16 | −12.79, −3.53 | <0.001 |
| OPN-375 372 μg | 112 | 49.57 (19.10) | 105 | −15.56 (1.96) | −8.01 | −13.36, −2.66 | 0.003 |
| OPN-375 186 μg | 113 | 50.29 (18.26) | 106 | −15.86 (1.95) | −8.31 | −13.65, −2.96 | 0.002 |
| Placebo | 114 | 49.41 (18.46) | 106 | −7.55 (1.95) | — | — | — |

Abbreviations:
AECRS = acute exacerbations of chronic rhinosinusitis;
BID = twice daily;
CFB = change from baseline;
CI = confidence interval;
CS = chronic sinusitis;
ET = early termination;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (4, 8, 12, 24/ET), treatment-by-week interaction, and continuous covariate: baseline score, with baseline score-by-week interaction; treatment-by-subgroup, subgroup-by-week, and treatment-by-subgroup-by-week interaction.

For the population using standard-delivery nasal steroids at trial entry, the baseline mean SNOT-22 total scores were similar across treatment groups. From baseline, all active treatment groups improved more than the minimum clinically important difference, though the placebo group did not (Table 36). Improvement with OPN-375 relative to placebo was nominally statistically significant (P<0.001) for the all OPN-375 group and for each individual dose group, with the magnitude of change similar for each OPN-375 dose group.

TABLE 36
Sinonasal Outcome Test-22 Total Score in Patients Entering Trial on Standard-
Delivery Nasal Steroid (Change from Baseline to Week 24/ET).

| | | | Week 24/ET | | | | |
|---|---|---|---|---|---|---|---|
| | Baseline | | | LSM CFB | | | |
| Treatment (BID) | n | Mean (SD) | n | (SE) | LSMD | 95% CI | P-value |

Primary Estimand

| | | | | | | | |
|---|---|---|---|---|---|---|---|
| All OPN-375 | 172 | 49.91 (18.60) | 158 | −18.02 (1.448) | −11.55 | −15.93, −7.17 | <0.001 |
| OPN-375 372 μg | 85 | 47.73 (18.62) | 74 | −17.64 (2.061) | −11.17 | −16.41, −5.94 | <0.001 |
| OPN-375 186 μg | 87 | 52.05 (18.43) | 84 | −18.40 (1.934) | −11.93 | −16.99, −6.86 | <0.001 |
| Placebo | 106 | 51.75 (19.33) | 95 | −6.47 (1.794) | — | — | — |

Abbreviations:
BID = twice daily;
CFB = change from baseline;
CI = confidence interval;
ET = early termination;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (4, 8, 12, 24/ET), treatment-by-week interaction, and continuous covariate: baseline score, with baseline score-by-week interaction.

In the population entering the study using a standard-delivery nasal steroid, baseline SNOT-22 total scores were similar across treatment groups for subjects with or without NP (Table 37). At 24 weeks, SNOT-22 total scores decreased (improved) from baseline in all treatment groups. OPN-375 treatment (all, low, and high dose) produced an improvement that was nominally statistically significantly greater than placebo in both CS patients with and without NP, with a similar magnitude of change between OPN-375 dose groups in both phenotypes.

TABLE 37
Sinonasal Outcome Test-22 Total Scores for Subjects Using Standard-Delivery
Nasal Steroid at Trial Entry by Phenotype (Change from Baseline to Week 24/ET).

| | | | Week 24/ET | | | | |
|---|---|---|---|---|---|---|---|
| | Baseline | | | LSM (SE) | | | |
| Treatment (BID) | n | Mean (SD) | n | CFB | LSMD | 95% CI | P-value |

CS Patients With Nasal Polyps

| | | | | | | | |
|---|---|---|---|---|---|---|---|
| All OPN-375 | 74 | 50.46 (18.99) | 66 | −20.07 (2.399) | −16.53 | −23.30, −9.77 | <0.001 |
| OPN-375 372 μg | 36 | 46.17 (18.48) | 29 | −19.94 (3.338) | −16.40 | −24.59, −8.22 | <0.001 |
| OPN-375 186 μg | 38 | 54.53 (18.80) | 37 | −20.20 (3.062) | −16.66 | −24.38, −8.95 | <0.001 |
| Placebo | 44 | 51.34 (21.44) | 38 | −3.54 (2.954) | — | — | — |

CS Patients Without Nasal Polyps

| | | | | | | | |
|---|---|---|---|---|---|---|---|
| All OPN-375 | 98 | 49.50 (18.38) | 92 | −16.77 (1.826) | −8.02 | −13.74, −2.30 | 0.006 |
| OPN-375 372 μg | 49 | 48.88 (18.83) | 45 | −16.46 (2.603) | −7.70 | −14.46, −0.94 | 0.026 |
| OPN-375 186 μg | 49 | 50.12 (18.09) | 47 | −17.09 (2.524) | −8.33 | −15.03, −1.63 | 0.015 |
| Placebo | 62 | 52.05 (17.85) | 57 | −8.76 (2.332) | — | — | — |

Abbreviations:
BID = twice daily;
CFB = change from baseline;
CI = confidence interval;
CS = chronic sinusitis;
ET = early termination;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (4, 8, 12, 24/ET), treatment-by-week interaction, and continuous covariate: baseline score, with baseline score-by-week interaction; treatment-by-subgroup, subgroup-by-week, and treatment-by-subgroup-by-week interaction.

Changes from baseline to Weeks 8 and 12 in the CSS reflected improvement relative to placebo for all 3 treatment groups, with the treatment difference for each active dose minus placebo nominally statistically significant in favor of active treatment at both Weeks 8 and 12 (Table 38). Thus, OPN-375 continued to provide benefit in subsequent weeks after symptomatic rescue medication was allowed.

The clinical response in symptom improvement that was observed in the primary endpoint (CSS) at Week 4 persisted throughout the period in which CSS was measured. Specifically, CSS at Week 8 and at Week 12 improved with OPN-375 (all and each dose) compared to placebo, with a magnitude of benefit over placebo that remained consistent over time (Table 38). The difference between active and placebo was nominally statistically significant for both individual doses, for CS patients with and without nasal polyps, and under both the primary and alternative primary estimand (Table 39). Similar to what was observed with other outcome measures, the treatment differences between OPN-375 and placebo were substantially larger using the alternative primary estimand (treating AECRS requiring antibiotics as one type of treatment failure) compared to other estimands.

TABLE 38
Composite Symptom Score through Week 12 (Chane from Baseline).

| | | | Week 8 | | | | Week 12 | | | |
|---|---|---|---|---|---|---|---|---|---|---|
| | Baseline | | | LSM (SE) | | | | LSM (SE) | | |
| Treatment (BID) | n | Mean | n | CFB | LSMD | P-value | n | CFB | LSMD | P-value |

Primary Estimand

| | | | | | | | | | | |
|---|---|---|---|---|---|---|---|---|---|---|
| All OPN-375 | 362 | 5.64 (1.720) | 350 | −2.04 (0.108) | −0.97 | <0.001 | 337 | −2.27 (0.115) | −0.81 | <0.001 |
| OPN-375 372 μg | 180 | 5.67 (1.750) | 172 | −2.14 (0.149) | −1.07 | <0.001 | 168 | −2.39 (0.157) | −0.93 | <0.001 |
| OPN-375 186 μg | 182 | 5.60 (1.695) | 178 | −1.95 (0.146) | −0.87 | <0.001 | 169 | −2.16 (0.155) | −0.70 | <0.001 |
| Placebo | 185 | 5.92 (1.778) | 173 | −1.08 (0.147) | — | — | 168 | −1.46 (0.156) | — | — |

Alternative primary estimand—Intercurrent Events Include Antibiotics for Acute
Exacerbations of CRS

| | | | | | | | | | | |
|---|---|---|---|---|---|---|---|---|---|---|
| All OPN-375 | 362 | 5.64 (1.720) | 350 | −1.94 (0.121) | −1.19 | <0.001 | 337 | −2.08 (0.131) | −1.08 | <0.001 |
| OPN-375 372 μg | 180 | 5.67 (1.750) | 172 | −2.00 (0.166) | −1.25 | <0.001 | 168 | −2.13 (0.181) | −1.14 | <0.001 |
| OPN-375 186 μg | 182 | 5.60 (1.695) | 178 | −1.88 (0.164) | −1.13 | <0.001 | 169 | −2.03 (0.179) | −1.03 | <0.001 |
| Placebo | 185 | 5.92 (1.778) | 175 | −0.75 (0.164) | — | — | 170 | −0.99 (0.178) | — | — |

Abbreviations:
BID = twice daily;
CFB = change from baseline;
CI = confidence interval;
CRS = chronic rhinosinusitis;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week (4, 8, 12), treatment-by-week interaction, and continuous covariate: baseline 7-day average score, with baseline score-by-week interaction.

TABLE 39
Composite Symptom Score through Week 12 by Phenotype.

| | | | Week 8 | | | | Week 12 | | | |
|---|---|---|---|---|---|---|---|---|---|---|
| | Baseline | | | LSM (SE) | | | | LSM (SE) | | |
| Treatment (BID) | n | Mean | n | CFB | LSMD | P-value | n | CFB | LSMD | P-value |

Primary Estimand
CS Patients With Nasal Polyps

| | | | | | | | | | | |
|---|---|---|---|---|---|---|---|---|---|---|
| All OPN-375 | 137 | 5.32 (1.802) | 131 | −2.25 (0.187) | −1.33 | <0.001 | 125 | −2.35 (0.198) | −0.97 | 0.002 |
| OPN-375 372 μg | 68 | 5.26 (1.800) | 63 | −2.40 (0.250) | −1.48 | <0.001 | 61 | −2.51 (0.266) | −1.14 | 0.001 |
| OPN-375 186 μg | 69 | 5.37 (1.814) | 68 | −2.10 (0.246) | −1.18 | <0.001 | 64 | −2.19 (0.261) | −0.81 | 0.021 |
| Placebo | 69 | 5.81 (1.841) | 64 | −0.93 (0.249) | — | — | 60 | −1.38 (0.265) | — | — |

CS Patients Without Nasal Polyps

| | | | | | | | | | | |
|---|---|---|---|---|---|---|---|---|---|---|
| All OPN-375 | 225 | 5.83 (1.643) | 219 | −1.88 (0.132) | −0.75 | <0.001 | 212 | −2.19 (0.140) | −0.71 | 0.003 |
| OPN-375 372 μg | 112 | 5.92 (1.678) | 109 | −1.96 (0.183) | −0.82 | 0.001 | 107 | −2.27 (0.195) | −0.79 | 0.003 |
| OPN-375 186 μg | 113 | 5.74 (1.610) | 110 | −1.81 (0.182) | −0.68 | 0.008 | 105 | −2.10 (0.194) | −0.62 | 0.022 |
| Placebo | 116 | 5.99 (1.744) | 109 | −1.14 (0.182) | — | — | 108 | −1.48 (0.193) | — | — |

Alternative primary estimand—Intercurrent Events Include Antibiotics for Acute Exacerbations of CRS
CS Patients With Nasal Polyps

| | | | | | | | | | | |
|---|---|---|---|---|---|---|---|---|---|---|
| All OPN-375 | 137 | 5.32 (1.802) | 131 | −2.25 (0.208) | −1.59 | <0.001 | 125 | −2.31 (0.225) | −1.27 | <0.001 |
| OPN-375 372 μg | 68 | 5.26 (1.800) | 63 | −2.46 (0.279) | −1.81 | <0.001 | 61 | −2.51 (0.304) | −1.46 | <0.001 |
| OPN-375 186 μg | 69 | 5.37 (1.814) | 68 | −2.03 (0.274) | −1.37 | <0.001 | 64 | −2.12 (0.298) | −1.07 | 0.008 |
| Placebo | 69 | 5.81 (1.841) | 65 | −0.65 (0.277) | — | — | 61 | −1.05 (0.302) | — | — |

CS Patients Without Nasal Polyps

| | | | | | | | | | | |
|---|---|---|---|---|---|---|---|---|---|---|
| All OPN-375 | 225 | 5.83 (1.643) | 219 | −1.71 (0.147) | −0.94 | <0.001 | 212 | −1.89 (0.160) | −0.97 | <0.001 |
| OPN-375 372 μg | 112 | 5.92 (1.678) | 109 | −1.68 (0.205) | −0.91 | 0.001 | 107 | −1.85 (0.224) | −0.93 | 0.003 |
| OPN-375 186 μg | 113 | 5.74 (1.610) | 110 | −1.74 (0.203) | −0.98 | <0.001 | 105 | −1.92 (0.223) | −1.00 | 0.001 |
| Placebo | 116 | 5.99 (1.744) | 110 | — | — | | 109 | −0.92 (0.221) | — | — |

Abbreviations:
BID = twice daily;
CFB = change from baseline;
CI = confidence interval;
CRS = chronic rhinosinusitis;
CS = chronic sinusitis;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol previous sinus surgery, nasal polyp status, treatment, week (4, 8, 12), treatment-by-week interaction, and continuous covariate: baseline 7-day average score, with baseline score-by-week interaction; treatment-by-subgroup, subgroup-by-week, and treatment-by-subgroup-by-week interaction.

There are 4 individual cardinal symptoms that define the diagnosis of chronic rhinosinusitis, with consensus publications requiring 2 of the 4 core symptoms in order to make the diagnosis, and some requiring that one of the symptoms must be either nasal congestion/obstruction or nasal discharge. All 4 individual symptoms improved from baseline more with OPN-375 compared to placebo at 4 weeks. The magnitude of improvement remained generally similar throughout the remainder of the measurement period (through 12 weeks), with nominal statistical differences between active and placebo at all time points in the all-active and high dose groups, and for all symptoms at all time points except facial pain/pressure at Week 12 for the low dose group (Table 40).

A similar response profile was found for analyses based on SE-2.

For CS patients without NP, the pattern of response was similar to the overall CS population under the primary estimand. All individual cardinal symptoms improved more with OPN-375 than with placebo at the primary Week 4 analysis timepoint, and the magnitude of improvement from baseline was sustained or increasing with OPN-375 over time through the last measurement at Week 12. Changes for the high-dose group were nominally significantly greater than with placebo for most symptoms at all time points, with the exception of facial pain/pressure, which was nominally significant in the high dose group at Week 8 but not Week 12. For the low dose groups, OPN-375 improvement was nominally statistically significantly greater than placebo for 3 symptoms at all time points. For facial pain/pressure, although the OPN-375 treatment effect increased over time, there was a sharply increased placebo response and the difference from placebo did not reach nominal statistical significance after Week 4 (Table 41). A similar profile was observed for subjects with NP and for analyses based on SE-2.

TABLE 40
Individual Cardinal Symptoms (Change from Baseline to Week 4, 8, and 12).

| | | | Week 4 | | | Week 8 | | | Week 12 | | |
|---|---|---|---|---|---|---|---|---|---|---|---|
| | | Baseline | | LSM | | | LSM | | | LSM | |
| Group | N | Score | n | CFB | LSMD | n | CFB | LSMD | n | CFB | LSMD |

Nasal Congestion/Obstruction

| | | | | | | | | | | | |
|---|---|---|---|---|---|---|---|---|---|---|---|
| All active | 362 | 2.14 | 357 | −0.64 | −0.39*** | 350 | −0.78 | −0.39*** | 337 | −0.85 | −0.36*** |
| 372 μg | 180 | 2.15 | 177 | −0.66 | −0.41*** | 172 | −0.81 | −0.43*** | 168 | −0.91 | −0.42*** |
| 186 μg | 182 | 2.13 | 180 | −0.62 | −0.37*** | 178 | −0.74 | −0.36*** | 169 | −0.79 | −0.30*** |
| Placebo | 185 | 2.25 | 180 | −0.25 | — | 173 | −0.38 | — | 168 | −0.49 | — |

Nasal Discharge (Rhinorrhea)

| | | | | | | | | | | | |
|---|---|---|---|---|---|---|---|---|---|---|---|
| All active | 362 | 1.89 | 357 | −0.55 | −0.34*** | 350 | −0.67 | −0.35*** | 337 | −0.76 | −0.31*** |
| 372 μg | 180 | 1.89 | 177 | −0.56 | −0.35*** | 172 | −0.69 | −0.37*** | 168 | −0.79 | −0.34*** |
| 186 μg | 182 | 1.90 | 180 | −0.54 | −0.33*** | 178 | −0.64 | −0.32*** | 169 | −0.72 | −0.28*** |
| Placebo | 185 | 2.01 | 180 | −0.21 | — | 173 | −0.32 | — | 168 | −0.45 | — |

Facial Pain or Pressure

| | | | | | | | | | | | |
|---|---|---|---|---|---|---|---|---|---|---|---|
| All active | 362 | 1.60 | 357 | −0.45 | −0.20*** | 350 | −0.60 | −0.22*** | 337 | −0.66 | −0.14 |
| 372 μg | 180 | 1.63 | 177 | −0.49 | −0.23*** | 172 | −0.64 | −0.26*** | 168 | −0.69 | −0.17* |
| 186 μg | 182 | 1.58 | 180 | −0.42 | −0.17* | 178 | −0.56 | −0.18* | 169 | −0.64 | −0.11 |
| Placebo | 185 | 1.66 | 180 | −0.25 | — | 173 | −0.38 | — | 168 | −0.52 | — |

Sense of Smell

| | | | | | | | | | | | |
|---|---|---|---|---|---|---|---|---|---|---|---|
| All active | 362 | 2.14 | 357 | −0.30 | −0.20*** | 350 | −0.46 | −0.28*** | 337 | −0.53 | −0.25*** |
| 372 μg | 180 | 2.13 | 177 | −0.32 | −0.22*** | 172 | −0.49 | −0.31*** | 168 | −0.54 | −0.26** |
| 186 μg | 182 | 2.15 | 180 | −0.28 | −0.18** | 178 | −0.44 | −0.26*** | 169 | −0.52 | −0.24** |
| Placebo | 185 | 2.25 | 180 | −0.10 | — | 173 | −0.18 | — | 168 | −0.28 | — |

Abbreviations:
CFB = change from baseline;
LSM = least squares mean;
LSMD = least squares mean difference (active − placebo);
MMRM = mixed model for repeated measures;
N = number of subjects;
n = number of subjects in subset;
— = not applicable.
***P ≤ 0.001;
**P < 0.01;
*P < 0.05
Note:
Inferential statistics are based on a linear MMRM including categorical effects for protocol, previous sinus surgery, nasal polyp status, treatment, week (4, 8, 12), treatment-by-week interaction, and continuous covariate: baseline 7-day average score, with baseline score-by-week interaction.

TABLE 41
Individual Cardinal Symptoms for CS Patients without Nasal Polyps (Change
from Baseline to Weeks 4, 8, and 12).

| | | | Week 4 | | | Week 8 | | | Week 12 | | |
|---|---|---|---|---|---|---|---|---|---|---|---|
| | | Baseline | | LSM | | | LSM | | | LSM | |
| Group | N | Score | n | CFB | LSMD | n | CFB | LSMD | n | CFB | LSMD |

Nasal Congestion/Obstruction

| | | | | | | | | | | | |
|---|---|---|---|---|---|---|---|---|---|---|---|
| All active | 225 | 2.16 | 222 | −0.60 | −0.35*** | 219 | −0.72 | −0.33*** | 212 | −0.81 | −0.30*** |
| 372 μg | 112 | 2.21 | 110 | −0.62 | −0.37*** | 109 | −0.74 | −0.35*** | 107 | −0.86 | −0.35*** |
| 186 μg | 113 | 2.11 | 112 | −0.58 | −0.33*** | 110 | −0.70 | −0.31*** | 105 | −0.75 | −0.24* |
| Placebo | 116 | 2.23 | 112 | −0.25 | — | 109 | −0.38 | — | 108 | −0.51 | — |

Nasal Discharge (Rhinorrhea)

| | | | | | | | | | | | |
|---|---|---|---|---|---|---|---|---|---|---|---|
| All active | 225 | 1.98 | 222 | −0.52 | −0.29*** | 219 | −0.62 | −0.24** | 212 | −0.74 | −0.28*** |
| 372 μg | 112 | 1.99 | 110 | −0.52 | −0.30*** | 109 | −0.64 | −0.27** | 107 | −0.76 | −0.30** |
| 186 μg | 113 | 1.97 | 112 | −0.51 | −0.29* | 110 | −0.59 | −0.22* | 105 | −0.72 | −0.26* |
| Placebo | 116 | 1.99 | 112 | −0.22 | — | 109 | −0.38 | — | 108 | −0.46 | — |

Facial Pain or Pressure

| | | | | | | | | | | | |
|---|---|---|---|---|---|---|---|---|---|---|---|
| All active | 225 | 1.69 | 222 | −0.43 | −0.20** | 219 | −0.55 | −0.17 | 212 | −0.64 | −0.12 |
| 372 μg | 112 | 1.72 | 110 | −0.45 | −0.22* | 109 | −0.58 | −0.20* | 107 | −0.66 | −0.14 |
| 186 μg | 113 | 1.65 | 112 | −0.41 | −0.18* | 110 | −0.52 | −0.14 | 105 | −0.63 | −0.11 |
| Placebo | 116 | 1.76 | 112 | −0.23 | — | 109 | −0.39 | — | 108 | −0.52 | — |

Sense of Smell

| | | | | | | | | | | | |
|---|---|---|---|---|---|---|---|---|---|---|---|
| All active | 225 | 2.00 | 222 | −0.37 | −0.22*** | 219 | −0.54 | −0.31*** | 212 | −0.60 | −0.26** |
| 372 μg | 112 | 2.08 | 110 | −0.38 | −0.23** | 109 | −0.57 | −0.34*** | 107 | −0.62 | −0.28** |
| 186 μg | 113 | 1.92 | 112 | −0.36 | −0.21** | 110 | −0.51 | −0.27** | 105 | −0.57 | −0.23* |
| Placebo | 116 | 2.18 | 112 | −0.15 | — | 109 | −0.23 | — | 108 | −0.34 | — |

Abbreviations:
CFB = change from baseline;
CS = chronic sinusitis;
LSM = least squares mean;
LSMD = least squares mean difference (active − placebo);
= not applicable.
***P≤ 0.001;
**P < 0.01;
*P < 0.05
Note:
Inferential statistics are based on a linear including categorical effects for protocol, previous sinus surgery, nasal polyp status, treatment, week (4, 8, 12), treatment-by-week interaction, and continuous covariate: baseline 7-day average score, with baseline score-by-week interaction; treatment-by-subgroup, subgroup-by-week, and treatment-by-subgroup-by-week interaction.

Change from baseline to Week 24/ET in Pittsburgh Sleep Quality Index (PSQI) Global score for the overall population of the pooled data set from the 2 pivotal studies was analyzed using the primary MMRM model, which included a categorical effect for protocol, and the repeated measures included change from baseline scores at Week 12 and 24/ET. At baseline, mean PSQI Global scores were similar across treatment groups and greater than 5, reflecting clinically significant disturbance in sleep quality of subjects (Table 42). PSQI Global scores improved (decreased) more in patients treated with OPN-375 than in patients receiving the placebo comparator, with the difference reaching nominal statistical significance for all OPN-375 patients and for each dose group.

TABLE 42
Pittsburgh Sleep Quality Index Global Score (Change from Baseline to Week 24/ET).

| | | | Week 24/ET | | | | |
|---|---|---|---|---|---|---|---|
| | Baseline | | | LSM (SE) | | | |
| Treatment (BID) | n | Mean (SD) | n | CFB | LSMD | 95% CI | P-value |
| All OPN-375 | 360 | 7.6 (3.66) | 333 | −1.49 (0.161) | −0.71 | (−1.22, −0.21) | 0.006 |
| OPN-375 372 μg | 180 | 7.5 (3.81) | 166 | −1.49 (0.220) | −0.71 | (−1.30, −0.13) | 0.017 |
| OPN-375 186 μg | 180 | 7.7 (3.50) | 167 | −1.49 (0.219) | −0.71 | (−1.30, −0.13) | 0.017 |
| Placebo | 184 | 7.1 (3.70) | 161 | −0.78 (0.221) | — | — | — |

Abbreviations:
BID = twice daily;
CFB = change from baseline;
CI = confidence interval;
ET = early termination;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week, treatment-by-week interaction, and continuous baseline score covariate, with baseline score-by-week interaction.

For CS patients without NP, treatment response was similar to the overall CS population under the primary estimand. The magnitude of improvement for patients treated with OPN-375 compared to placebo was similar for CS patients with (−0.66) or without (−0.74) polyps in the nasal cavity. Nominal statistical significance versus placebo was reached for all OPN-375 treated patients without NP, but not for patients with NP, and there was no obvious dose pattern (Table 43).

TABLE 43
Pittsburgh Sleep Quality Index Global Score by Phenotype (Change from
Baseline to Week 24/ET).

| | | | Week 24/ET | | | | |
|---|---|---|---|---|---|---|---|
| | Baseline | | | LSM (SE) | | | |
| Treatment (BID) | n | Mean (SD) | n | CFB | LSMD | 95% CI | P-value |

CS Patients With Nasal Polyps

| | | | | | | | |
|---|---|---|---|---|---|---|---|
| All OPN-375 | 136 | 7.7 (3.78) | 122 | −1.58 (0.283) | −0.66 | (−1.51, 0.18) | 0.122 |
| OPN-375 372 μg | 68 | 7.4 (3.87) | 59 | −1.99 (0.378) | −1.07 | (−2.04, −0.09) | 0.032 |
| OPN-375 186 μg | 68 | 8.0 (3.68) | 63 | −1.19 (0.369) | −0.26 | (−1.22, 0.70) | 0.593 |
| Placebo | 69 | 7.3 (3.88) | 58 | −0.93 (0.380) | — | — | — |

CS Patients Without Nasal Polyps

| | | | | | | | |
|---|---|---|---|---|---|---|---|
| All OPN-375 | 224 | 7.5 (3.59) | 211 | −1.44 (0.196) | −0.74 | (−1.38, −0.11) | 0.022 |
| OPN-375 372 μg | 112 | 7.5 (3.79) | 107 | −1.21 (0.268) | −0.51 | (−1.25, 0.22) | 0.168 |
| OPN-375 186 μg | 112 | 7.5 (3.39) | 104 | −1.67 (0.272) | −0.97 | (−1.71, −0.24) | 0.010 |
| Placebo | 115 | 7.0 (3.60) | 103 | −0.69 (0.272) | — | — | — |

Abbreviations:
BID = twice daily;
CFB = change from baseline;
CI = confidence interval;
CS = chronic sinusitis;
ET = early termination;
LSM = least squares mean;
LSMD = least squares mean difference (active − placebo);
MMRM = mixed model for repeated measures;
SD = standard deviation;
SE = standard error
Note:
Inferential statistics are based on a linear MMRM including categorical effects for protocol, previous sinus surgery, nasal polyp status, treatment, week, treatment-by-week interaction, and continuous baseline score covariate, with baseline score-by-week interaction; treatment-by-subgroup, subgroup-by-week, and treatment-by-subgroup-by-week interaction.

PGIC is a global patient-reported outcome measure, and indicative of patient perception of change in their disease with treatment. Over 80% of CS patients treated with OPN-375 reported improvement. For the FAS for subpopulations of CS patients either with or without NP, the proportion of improved patients was nominally statistically significantly greater with OPN-375 than with placebo for all OPN-375 patients and in each individual dose group (Table 44).

TABLE 44
Patient Global Impressions of Change at Week 24/ET

| | | | | CS Patients With | | | CS Patients Without | | |
|---|---|---|---|---|---|---|---|---|---|
| | Full Analysis Set | | | Nasal Polyps | | | Nasal Polyps | | |
| | | OPN | OPN | | OPN | OPN | | OPN- | OPN- |
| | | 375 | 375 | | 375 | 375 | | 375 | 375 |
| | Placebo | 186 μg | 372 μg | Placebo | 186 μg | 372 μg | Placebo | 186 μg | 372 μg |
| Category | (N = 185) | (N = 182) | (N = 180) | (N = 69) | (N = 69) | (N = 68) | (N = 116) | (N = 113) | (N = 112) |
| n | 163 | 169 | 167 | 58 | 63 | 60 | 105 | 106 | 107 |
| Improved | 98 (60.1) | 137 (81.1) | 143 (85.6) | 30 (51.7) | 51 (81.0) | 51 (85.0) | 68 (64.8) | 86 (81.1) | 92 (86.0) |
| Very much | 7 (4.3) | 29 (17.2) | 27 (16.2) | 3 (5.2) | 5 (7.9) | 8 (13.3) | 4 (3.8) | 24 (22.6) | 19 (17.8) |
| improved | | | | | | | | | |
| Much | 34 (20.9) | 59 (34.9) | 65 (38.9) | 10 (17.2) | 24 (38.1) | 25 (41.7) | 24 (22.9) | 35 (33.0) | 40 (37.4) |
| improved | | | | | | | | | |
| Minimally | 57 (35.0) | 49 (29.0) | 51 (30.5) | 17 (29.3) | 22 (34.9) | 18 (30.0) | 40 (38.1) | 27 (25.5) | 33 (30.8) |
| improved | | | | | | | | | |
| Not Improved | 65 (39.9) | 32 (18.9) | 24 (14.4) | 28 (48.3) | 12 (19.0) | 9 (15.0) | 37 (35.2) | 20 (18.9) | 15 (14.0) |
| Odds Ratio | — | 2.88 | 4.01 | — | 3.89 | 5.24 | — | 2.39 | 3.42 |
| (Improved) | | | | | | | | | |
| P-value | — | <0.001 | <0.001 | — | 0.001 | <0.001 | — | 0.007 | <0.001 |

Abbreviations:
CS = chronic sinusitis;
ET = early termination;
MMRM = mixed model for repeated measures
Note:
The denominator for each visit is the number of subjects with an assessment at the visit. Inferential statistics are based on a logistic MMRM including categorical effects for protocol, previous sinus surgery, nasal polyp status, treatment, week (4, 24/ET), and treatment-by-week interaction.

Using data from all patients, irrespective of treatment group, the mean change in the average of the percentages of opacified volume in the sinus cavities (APOV) for patients reporting each PGIC score (both at Week 24) is presented in Table 45. There is a linear relationship, extending in both positive and negative directions, between patient-reported change in their disease and sinus opacification, suggesting that these outcomes have some degree of association. The “no change” level of patient-reported outcome was associated with almost no change in APOV, suggesting that the outcomes are also similarly centered. This suggests that the global patient-reported scores can be used to help evaluate the clinical meaning of change in APOV, at least at the population level.

The mean APOV improvement in patients reporting that they are minimally clinically improved was −2.86%, suggesting that a population with at least this degree of improvement can be considered to have improved by an amount that is meaningful to patients who have the disease. Similarly, a mean APOV improvement of −7.26% was observed in patients reporting that they were “much improved” from baseline. Thus, in the population of patients entering these studies with at least moderate baseline symptoms and an average baseline APOV of ˜65%, a mean improvement of ≥2.86% in APOV corresponds with “clinically meaningful” disease improvement that is perceived by the patient and a mean improvement of ≥7.26% corresponds with much clinical improvement.

TABLE 45
Mean Change in Average of Percentages of Opacified Volume in
Ethmoid and Maxillary Sinuses from Baseline to Week 24/ET by
Patient-Reported Global Change Score at Week 24/ET.

| | PGIC Category | | | | | | |
|---|---|---|---|---|---|---|---|
| | Very | | | | | | Very |
| | Much | Much | Minimally | No | Minimally | Much | Much |
| Statistic | Improved | Improved | Improved | Change | Worsened | Worsened | Worsened |
| n | 67 | 164 | 164 | 90 | 16 | 10 | 4 |
| Mean | −10.53% | −7.26% | −2.86% | −0.32% | 2.01% | 4.82% | 5.30% |
| Change | | | | | | | |

Abbreviations: APOV = average of the percentages of opacified volume; ET = early termination; FAS = Full Analysis Set; PGIC = patient-reported global change
Note:
FAS subjects from Protocols OPN-FLU-CS-3205 and Protocol OPN-FLU-CS-3206 with complete data for APOV and PGIC, i.e., subjects with a change from baseline value for APOV and a value for PGIC at Week 24/ET represent the pool. Missing data was ignored in this analysis.

For CS patients without polyps in the nasal cavity, more patients receiving OPN-375 than placebo had a degree of objective improvement in APOV associated with clinically meaningful improvement. The overall percent of patients observed on CT scan to have APOV changes exceeding the clinically meaningful threshold was similar for treatment groups without NP (53%) and with NP (56%), and both groups had greater response rates than placebo, though the placebo response rate was higher (8.5%) for CS patients without NP, reducing the placebo-subtracted difference and nominal statistical significance in those groups (Table 46).

TABLE 46
Clinically Meaningful Within-Subject Improvement in Average of
Percentages of Opacified Volume in Ethmoid and Maxillary Sinuses
at Week 24/ET.

| | | OPN-375 | OPN-375 | OPN-375 |
|---|---|---|---|---|
| | Placebo | 186 μg | 186 μg | Total |
| Overall, N | 185 | 182 | 180 | 362 |
| Yes, n (%) | 62 (37.6) | 93 (55.4) | 90 (52.9) | 183 (54.1) |
| Odds Ratio | — | 1.89 | 1.73 | 1.81 |
| P-value | — | 0.005 | 0.014 | 0.002 |
| CS Patients With Nasal | 69 | 69 | 68 | 137 |
| Polyps, N | | | | |
| Yes | 20 (32.3) | 40 (63.5) | 31 (49.2) | 71 (56.3) |
| Odds Ratio | — | 3.10 | 1.78 | 2.35 |
| P-value | — | 0.002 | 0.113 | 0.007 |
| CS Patients Without | 116 | 113 | 112 | 225 |
| Nasal Polyps, N | | | | |
| Yes | 42 (40.8) | 53 (50.5) | 59 (55.1) | 112 (52.8) |
| Odds Ratio | | 1.41 | 1.71 | 1.55 |
| P-value | | 0.222 | 0.055 | 0.071 |

Abbreviations:
CS = chronic sinusitis;
ET = early termination

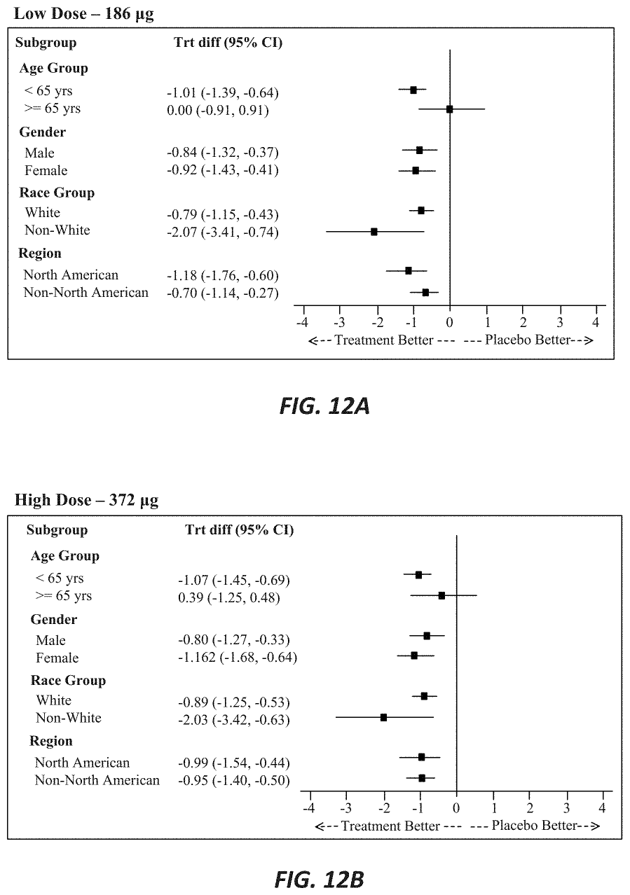

For each coprimary endpoint, subgroup analyses were performed for the FAS of the pooled data based on age (<65 years vs ≥65 years), gender (male vs female), race (White vs Non-White) and region (North America vs Non-North America). In general, each these subgroups showed point-estimate evidence of improvement on both endpoints through the sample sizes, nominal statistical significance, and magnitude of the improvement varied by subgroup. For the instantaneous AM CSS coprimary endpoint, LS mean changes were of greater magnitude with active treatment, and treatment differences reflected nominally statistically significant improvement relative to placebo for each subgroup examined, with the exception of subjects aged ≥65 years (FIGS. 12A-12B).

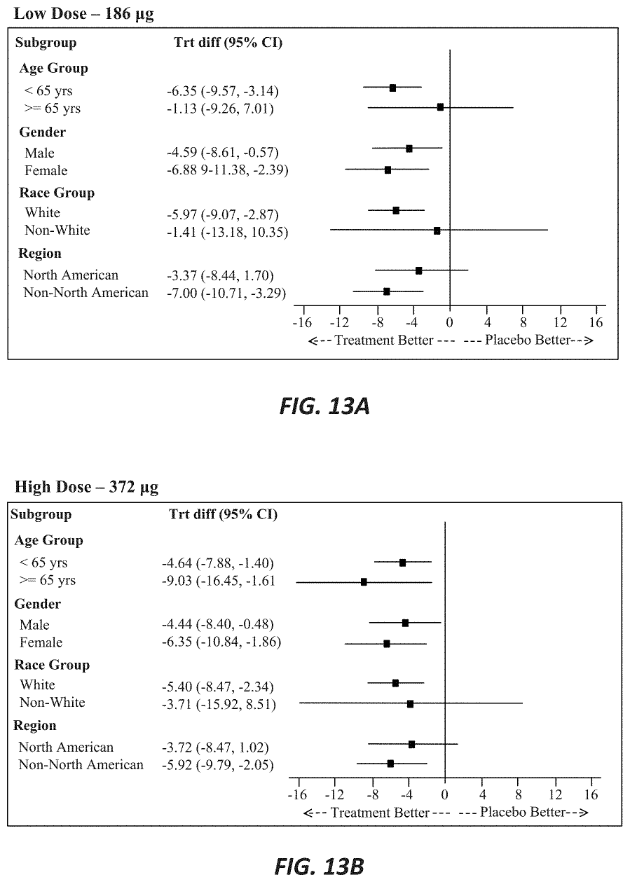

Similarly, changes from baseline to Week 24/ET in APOV showed nominally statistically significant treatment differences in favor of active treatment for most subgroups (FIGS. 13A-13B). The noted exceptions were for the following subgroups: age 65 years (low dose), non-White subjects (both dose groups), and those enrolled in North America (both dose groups). The lack of nominal statistical significance of treatment differences in certain comparisons for the co-primary endpoint measures for the subgroups as noted above could reflect substantially smaller sample sizes.

Based on co-primary endpoint, key secondary endpoint and an array of other secondary endpoint efficacy data from the studies and the integrated analysis of pooled data, both the 186 μg and 372 μg OPN-375 doses demonstrated activity in treatment of chronic sinusitis. The magnitude of treatment differences observed for each dose were similar; although the higher dose was associated with numerically greater improvement in composite symptoms, individual symptoms, acute exacerbation rates, and the proportion of patients reporting symptomatic improvement, these differences were small.

The consistency of effects observed between instantaneous and reflective AM/PM CSS assessments as recorded prior to the AM/PM doses, respectively, in each of the pivotal studies in patients with CS, demonstrate that efficacy is continuously sustained over a 24-hour period, supporting BID dose administration (Table 47) Considering the totality of data, the recommended dosage of OPN-375 for the treatment of CS is 1 or 2 actuations (93 μg of fluticasone propionate per spray) in each nostril BID for a total daily dose of fluticasone propionate of 372 or 744 μg, respectively.

TABLE 47
Changes from Baseline to End of Week 4 in Instantaneous AM/PM and
Reflective AM/PM Composite Symptom Scores

| | Study A | | | | | Study B | | | | |
|---|---|---|---|---|---|---|---|---|---|---|
| | | Baseline | | LSM | | | Baseline | | LSM | |
| | | Score | | (SE) | | | Score | | (SE) | |
| Group | N | Mean (SD) | n | CFB | LSMD | N | Mean (SD) | n | CFB | LSMD |

Instantaneous AM

| | | | | | | | | | | |
|---|---|---|---|---|---|---|---|---|---|---|
| 372 μg | 107 | 5.48 | 106 | −1.60 | −0.98*** | 73 | 5.97 | 71 | −1.74 | −0.93*** |
| | | (1.833) | | (0.163) | | | (1.588) | | (0.204) | |
| 186 μg | 110 | 5.42 | 108 | −1.58 | −0.97*** | 72 | 5.87 | 72 | −1.54 | −0.73* |
| | | (1.809) | | (0.161) | | | (1.476) | | (0.204) | |
| Placebo | 110 | 5.77 | 108 | −0.62 | — | 75 | 6.15 | 72 | −0.81 | — |
| | | (1.775) | | (0.161) | | | (1.768) | | (0.202) | |

Instantaneous PM

| | | | | | | | | | | |
|---|---|---|---|---|---|---|---|---|---|---|
| 372 μg | 107 | 5.22 | 105 | −1.52 | −0.97*** | 73 | 5.73 | 72 | −1.82 | −1.04*** |
| | | (1.955) | | (0.172) | | | (1.578) | | (0.211) | |
| 186 μg | 110 | 5.23 | 108 | −1.44 | −0.89*** | 72 | 5.69 | 72 | −1.62 | −0.85** |
| | | (1.826) | | (0.169) | | | (1.577) | | (0.210) | |
| Placebo | 110 | 5.55 | 108 | −0.55 | — | 75 | 5.97 | 73 | −0.78 | — |
| | | (1.801) | | (0.170) | | | (1.847) | | (0.208) | |

Reflective AM

| | | | | | | | | | | |
|---|---|---|---|---|---|---|---|---|---|---|
| 372 μg | 107 | 5.28 | 106 | −1.59 | −0.99*** | 73 | 5.85 | 71 | −1.79 | −0.97*** |
| | | (1.897) | | (0.168) | | | (1.567) | | (0.207) | |
| 186 μg | 110 | 5.27 | 108 | −1.49 | −0.89*** | 72 | 5.86 | 72 | −1.60 | −0.77** |
| | | (1.825) | | (0.166) | | | (1.455) | | (0.206) | |
| Placebo | 110 | 5.73 | 108 | −0.60 | — | 75 | 6.04 | 72 | −0.82 | — |
| | | (1.759) | | (0.168) | | | (1.745) | | (0.205) | |

Reflective PM

| | | | | | | | | | | |
|---|---|---|---|---|---|---|---|---|---|---|
| 372 μg | 107 | 5.35 | 105 | −1.61 | −1.01*** | 73 | 5.74 | 72 | −1.88 | −1.07*** |
| | | (1.932) | | (0.172) | | | (1.531) | | (0.209) | |
| 186 μg | 110 | 5.34 | 108 | −1.49 | −0.89*** | 72 | 5.77 | 72 | −1.53 | −0.73* |
| | | (1.830) | | (0.169) | | | (1.469) | | (0.208) | |
| Placebo | 110 | 5.62 | 108 | −0.61 | — | 75 | 5.97 | 73 | −0.81 | — |
| | | (1.790) | | (0.170) | | | (1.817) | | (0.206) | |

Abbreviations: AM = morning; CFB = change from baseline; LSM = least squares mean; LSMD = least squares mean difference (active - placebo); MMRM = mixed model for repeated measures; N = number of subjects; n = number of subjects in subset; PM = evening; SD = standard deviation; SE = standard error; — = not applicable.
***P ≤ 0.001; **P < 0.01; *P < 0.05
Note:
Inferential statistics are based on a linear MMRM including categorical effects for protocol, previous sinus surgery, nasal polyp status, treatment, week (4, 8, 12), treatment-by-week interaction, and continuous covariate: baseline 7-day average score, with baseline score-by-week interaction.

Each of the pivotal studies was 6 months in duration, and several efficacy outcome measures were assessed over time and provide information regarding the persistence of the effect of OPN-375 over this period.

The magnitude of improvement from baseline in composite symptom score with OPN-375 treatment remains similar or slightly increases over time as treatment extends past the primary endpoint at 4 weeks through the entire measurement period (through Week 12). A sustained or increasing treatment effect is observed for each phenotype. In addition, individual symptom scores were assessed during this period and showed a similar pattern of persistent efficacy throughout the measurement period.

Persistence of response over time may also be informed by the disease-specific quality of life instrument SNOT-22, measuring a range of symptoms and functional patient-reported domains. SNOT-22 was obtained at timepoints through Week 24. As with composite symptom score, the initially observed improvement at Week 4 was sustained through the measurement period (to Week 24) and, at all timepoints, the improvement from baseline was greater than the minimally clinical important difference for the SNOT-22 measurement instrument (i.e., 8.9 points) (Table 48). Improvement in symptoms and quality of life are the primary objectives for treatment of CS, and the sustained, and in some instances, increased treatment effects observed in these domains suggest not only a lack of treatment tolerance but that a longer duration of therapy may lead to greater benefit in some domains within the time frames studied.

The PGIC captured the most direct global assessment of patient-perceived treatment benefit (or harm). In these pivotal studies, this outcome also demonstrated that a treatment benefit was observed through Week 24 (Table 49). Specifically, in the integrated dataset, a higher percentage of subjects perceived improvement of symptoms with active treatment compared to placebo at both Week 4 and Week 24/ET of the double-blind treatment phase (Table 50).

Taken together, these data support the conclusion that there is a sustained treatment effect of OPN-375 through 6 months of treatment.

TABLE 48
Change from Baseline over Time in Sinonasal Outcome Test-22 Total Scores.

| OPN- | | | | | | | | |
|---|---|---|---|---|---|---|---|---|
| 375 | Week 4 | | Week 8 | | Week 12 | | Week 24 | |
| Dose | 186 μg | 372 μg | 186 μg | 372 μg | 186 μg | 372 μg | 186 μg | 372 μg |

Study A

| | | | | | | | | |
|---|---|---|---|---|---|---|---|---|
| LSM | −13.27 | −16.25 | −16.87 | −18.30 | −16.16 | −19.74 | −18.05 | −22.77 |
| CFB | | | | | | | | |
| LSMD | −5.70 | −8.68 | −7.46 | −8.89 | −5.71 | −9.29 | −7.89 | −12.61 |
| 95% | −10.03, | −13.05, | −11.90, | −13.35, | −10.58, | −14.18, | −12.57, | −17.32, |
| CI | −1.37 | −4.31 | −3.03 | −4.44 | −0.83 | −4.41 | −3.21 | −7.90 |
| P- | 0.010 | <0.001 | 0.001 | <0.001 | 0.022 | <0.001 | 0.001 | <0.001 |
| value | | | | | | | | |

Study B

| | | | | | | | | |
|---|---|---|---|---|---|---|---|---|
| LSM | −9.16 | −13.01 | −14.29 | −13.87 | −16.19 | −15.53 | −19.45 | −15.54 |
| CFB | | | | | | | | |
| LSMD | −2.65 | −6.50 | −8.65 | −8.24 | −7.51 | −6.85 | −10.72 | −6.81 |
| 95% | −7.43, | −11.29, | −14.10, | −13.71, | −13.10, | −12.46, | −16.79, | −12.92, |
| CI | 2.13 | −1.71 | −3.20 | −2.77 | −1.93 | −1.24 | −4.65 | −0.71 |
| P- | 0.276 | 0.008 | 0.002 | 0.003 | 0.009 | 0.017 | <0.001 | 0.029 |
| value | | | | | | | | |

Abbreviations: CFB = change from baseline; CI = confidence interval; LSM = least squares mean; LSMD = least squares mean difference (active - placebo)

TABLE 49
Patient Global Impression of Change Over Time.

| | | Study A | | | Study B | | |
|---|---|---|---|---|---|---|---|
| | | | OPN | OPN | | OPN- | OPN- |
| | | | 375 | 375 | | 375 | 375 |
| | | Placebo | 186 μg | 372 μg | | 186 μg | 372 μg |
| | | (N = | (N = | (N = | Placebo | (N = | (N = |
| Time Point | Category | 110) | 110) | 107) | (N = 75) | 72) | 73) |
| Week 4 | n | 104 | 109 | 103 | 72 | 71 | 71 |
| | Improved | 57 | 84 | 77 | 37 | 47 | 56 |
| | | (54.8) | (77.1) | (74.8) | (51.4) | (66.2) | (78.9) |
| | Very much | 0 | 8 | 11 | 0 | 6 | 4 |
| | improved | | (7.3) | (10.7) | | (8.5) | (5.6) |
| | Much improved | 7 | 29 | 28 | 8 | 22 | 24 |
| | | (6.7) | (26.6) | (27.2) | (11.1) | (31.0) | (33.8) |
| | Minimally | 50 | 47 | 38 | 29 | 19 | 28 |
| | improved | (48.1) | (43.1) | (36.9) | (40.3) | (26.8) | (39.4) |
| | Not Improved | 47 | 25 | 26 | 35 | 24 | 15 |
| | | (45.2) | (22.9) | (25.2) | (48.6) | (33.8) | (21.1) |
| | Odds Ratio | — | 2.80 | 2.50 | — | 1.85 | 3.52 |
| | (Improved) | | | | | | |
| | P-value | — | <0.001 | 0.003 | — | 0.078 | 0.001 |
| Week | n | 94 | 99 | 98 | 69 | 70 | 69 |
| 24/ET | | | | | | | |
| | Improved | 56 | 78 | 80 | 42 | 59 | 63 |
| | | (59.6) | (78.8) | (81.6) | (60.9) | (84.3) | (91.3) |
| | Very much | 4 | 10 | 15 | 3 | 19 | 12 |
| | improved | (4.3) | (10.1) | (15.3) | (4.3) | (27.1) | (17.4) |
| | Much | 20 | 34 | 38 | 14 | 25 | 27 |
| | improved | (21.3) | (34.3) | (38.8) | (20.3) | (35.7) | (39.1) |
| | Minimally | 32 | 34 | 27 | 25 | 15 | 24 |
| | improved | (34.0) | (34.3) | (27.6) | (36.2) | (21.4) | (34.8) |
| | Not Improved | 38 | 21 | 18 | 27 | 11 | 6 |
| | | (40.4) | (21.2) | (18.4) | (39.1) | (15.7) | (8.7) |
| | Odds Ratio | — | 2.61 | 3.10 | — | 3.36 | 6.81 |
| | (Improved) | | | | | | |
| | P-value | — | 0.004 | <0.001 | — | 0.004 | <0.001 |

Abbreviations: ET = early termination

TABLE 50
Patient Global Impression of Change Over Time.

| | | CS Patients With | | | CS Patients Without | | |
|---|---|---|---|---|---|---|---|
| | | Nasal Polyps | | | Nasal Polyps | | |
| | | | OPN | OPN | | OPN- | OPN- |
| | | | 375 | 375 | | 375 | 375 |
| | | | 186 μg | 372 μg | Placebo | 186 μg | 372 μg |
| | | Placebo | (N = | (N = | (N = | (N = | (N = |
| Time Point | Category | (N = 69) | 69) | 68) | 116) | 113) | 112) |
| Week 4 | n | 65 | 69 | 64 | 111 | 111 | 110 |
| | Improved | 35 | 56 | 48 | 59 | 75 | 85 |
| | | (53.8) | (81.2) | (75.0) | (53.2) | (67.6) | (77.3) |
| | Very much | 0 | 5 | 6 | 0 | 9 | 9 |
| | improved | | (7.2) | (9.4) | | (8.1) | (8.2) |
| | Much improved | 3 | 20 | 16 | 12 | 31 | 36 |
| | | (4.6) | (29.0) | (25.0) | (10.8) | (27.9) | (32.7) |
| | Minimally | 32 | 31 | 26 | 47 | 35 | 40 |
| | improved | (49.2) | (44.9) | (40.6) | (42.3) | (31.5) | (36.4) |
| | Not Improved | 30 | 13 | 16 | 52 | 36 | 25 |
| | | (46.2) | (18.8) | (25.0) | (46.8) | (32.4) | (22.7) |
| | Odds Ratio | — | 3.67 | 2.59 | — | 1.85 | 3.03 |
| | (Improved) | | | | | | |
| | P-value | — | 0.001 | 0.014 | — | 0.028 | <0.001 |
| Week | n | 58 | 63 | 60 | 105 | 106 | 107 |
| 24/ET | | | | | | | |
| | Improved | 30 | 51 | 51 | 68 | 86 | 92 |
| | | (51.7) | (81.0) | (85.0) | (64.8) | (81.1) | (86.0) |
| | Very much | 3 | 5 | 8 | 4 | 24 | 19 |
| | improved | (5.2) | (7.9) | (13.3) | (3.8) | (22.6) | (17.8) |
| | Much | 10 | 24 | 25 | 24 | 35 | 40 |
| | improved | (17.2) | (38.1) | (41.7) | (22.9) | (33.0) | (37.4) |
| | Minimally | 17 | 22 | 18 | 40 | 27 | 33 |
| | improved | (29.3) | (34.9) | (30.0) | (38.1) | (25.5) | (30.8) |
| | Not Improved | 28 | 12 | 9 | 37 | 20 | 15 |
| | | (48.3) | (19.0) | (15.0) | (35.2) | (18.9) | (14.0) |
| | Odds Ratio | — | 3.89 | 5.24 | — | 2.39 | 3.42 |
| | (Improved) | | | | | | |
| | P-value | — | 0.001 | <0.001 | — | 0.007 | <0.001 |

Abbreviations: CS = chronic sinusitis; ET = early termination; MMRM = mixed model for repeated measures
Note:
The denominator for each visit is the number of subjects with an assessment at the visit. Inferential statistics are based on a logistic MMRM including categorical effects for protocol, previous sinus surgery, nasal polyp status, treatment, week (4, 24/ET), treatment-by-week interaction, treatment-by-subgroup, subgroup-by-week, and treatment-by-subgroup-by-week interaction.

TABLE 51
Co-Primary Endpoint Analysis Comparison of Treatment Differences by
Population (Primary Estimand).

| Study A | | | | Study B (FAS = CRSsNP) | | | | Pooled Data | | | |
|---|---|---|---|---|---|---|---|---|---|---|---|
| | | 95% | P- | | | 95% | P- | | | 95% | P- |
| n | LSMD | CI | value | n | LSMD | CI | value | n | LSMD | CI | value |

Composite Symptom Score-Change from Baseline to End of Week 4
Full Analysis Set

| | | | | | | | | | | | | |
|---|---|---|---|---|---|---|---|---|---|---|---|---|
| All OPN- | 214 | −0.97 | −1.36, | <0.001 | 143 | −0.83 | −1.32, | <0.001 | 357 | −0.92 | −1.22, | <0.001 |
| 375 | | | −0.59 | | | | −0.35 | | | | −0.62 | |
| OPN-375 | 106 | −0.98 | −1.43, | <0.001 | 71 | −0.93 | −1.49, | 0.001 | 177 | −0.96 | −1.31, | <0.001 |
| 372 μg | | | −0.54 | | | | −0.37 | | | | −0.62 | |
| OPN-375 | 108 | −0.97 | −1.41, | <0.001 | 72 | −0.73 | −1.29, | 0.011 | 180 | −0.87 | −1.22, | <0.001 |
| 186 μg | | | −0.52 | | | | −0.17 | | | | −0.53 | |
| Placebo | 108 | — | — | — | 72 | — | — | — | 180 | — | — | — |

CS Patients Without Nasal Polyps

| | | | | | | | | | | | | |
|---|---|---|---|---|---|---|---|---|---|---|---|---|
| All OPN- | 80 | −0.80 | −1.43, | 0.013 | 143 | −0.83 | −1.32, | <0.001 | 222 | −0.81 | −1.19, | <0.001 |
| 375 | | | −0.17 | | | | −0.35 | | | | −0.43 | |
| OPN-375 | 40 | −0.77 | −1.50, | 0.038 | 71 | −0.93 | −1.49, | 0.001 | 110 | −0.86 | −1.30, | <0.001 |
| 372 μg | | | −0.04 | | | | −0.37 | | | | −0.42 | |
| OPN375 | 40 | −0.83 | −1.56, | 0.025 | 72 | −0.73 | −1.29, | 0.011 | 112 | −0.76 | −1.20, | <0.001 |
| 186 μg | | | −0.10 | | | | −0.17 | | | | −0.32 | |
| Placebo | 40 | — | — | — | 72 | — | — | — | 112 | — | — | — |

Average of Percentages of Opacified Volume in Ethmoid and Maxillary Sinuses - Change from Baseline to
Week 24/Early Termination
Full Analysis Set

| | | | | | | | | | | | | |
|---|---|---|---|---|---|---|---|---|---|---|---|---|
| All OPN- | 199 | −4.28 | −7.61, | 0.012 | 139 | −7.26 | −11.36, | <0.001 | 338 | −5.48 | −8.05, | <0.001 |
| 375 | | | −0.96 | | | | −3.16 | | | | −2.90 | |
| OPN-375 | 101 | −4.59 | −8.41, | 0.018 | 69 | −6.33 | −11.08, | 0.009 | 170 | −5.29 | −8.25, | <0.001 |
| 372 μg | | | −0.78 | | | | −1.58 | | | | −2.32 | |
| OPN-375 | 98 | −3.98 | −7.86, | 0.045 | 70 | −8.19 | −12.93, | <0.001 | 168 | −5.67 | −8.67, | <0.001 |
| 186 μg | | | −0.09 | | | | −3.45 | | | | −2.66 | |
| Placebo | 99 | — | — | — | 66 | — | — | — | 165 | — | — | — |

CS Patients Without Nasal Polyps

| | | | | | | | | | | | | |
|---|---|---|---|---|---|---|---|---|---|---|---|---|
| All OPN- | 73 | −0.21 | −5.70, | 0.939 | 139 | −7.26 | −11.36, | <0.001 | 212 | −4.76 | −8.03, | 0.004 |
| 375 | | | 5.27 | | | | −3.16 | | | | −1.48 | |
| OPN-375 | 38 | −2.40 | −8.66, | 0.451 | 69 | −6.33 | −11.08, | 0.009 | 107 | −4.95 | −8.70, | 0.010 |
| 372 μg | | | 3.85 | | | | −1.58 | | | | −1.19 | |
| OPN-375 | 35 | 1.98 | −4.42, | 0.545 | 70 | −8.19 | −12.93, | <0.001 | 105 | −4.57 | −8.39, | 0.019 |
| 186 μg | | | 8.38 | | | | −3.45 | | | | −0.75 | |
| Placebo | 37 | — | — | — | 66 | — | — | — | 103 | — | — | — |

Abbreviations: CI = confidence interval; CRSsNP = chronic rhinosinusitis without nasal polyps; FAS = Full Analysis Set; LSMD = least squares mean treatment difference.

TABLE 52
Co-Primary Endpoint Analysis Comparison by Population (Alternative primary estimand).

| Study A | | | | Study B (FAS = CRSSNP) | | | | Pooled Data | | | |
|---|---|---|---|---|---|---|---|---|---|---|---|
| | | 95% | P- | | | 95% | P- | | | 95% | P- |
| n | LSMD | CI | value | n | LSMD | CI | value | n | LSMD | CI | value |

Composite Symptom Score - Change from Baseline to End of Week 4
Full Analysis Set

| | | | | | | | | | | | | |
|---|---|---|---|---|---|---|---|---|---|---|---|---|
| All OPN- | 214 | −1.23 | −1.65, | <0.001 | 143 | −0.88 | −1.37, | <0.001 | 357 | −1.09 | −1.41, | <0.001 |
| 375 | | | −0.81 | | | | −0.38 | | | | −0.77 | |
| OPN-375 | 106 | −1.25 | −1.74, | <0.001 | 71 | −0.95 | −1.53, | 0.001 | 180 | −1.13 | −1.50, | <0.001 |
| 372 μg | | | −0.77 | | | | −0.38 | | | | −0.76 | |
| OPN-375 | 108 | −1.20 | −1.68, | <0.001 | 72 | −0.80 | −1.37, | 0.007 | 177 | −1.04 | −1.41, | <0.001 |
| 186 μg | | | −0.72 | | | | −0.23 | | | | −0.67 | |
| Placebo | 108 | — | — | — | 72 | — | — | — | 180 | — | — | — |

CS Patients Without Nasal Polyps

| | | | | | | | | | | | | |
|---|---|---|---|---|---|---|---|---|---|---|---|---|
| All OPN- | 80 | −1.16 | −1.85, | <0.001 | 143 | −0.88 | −1.37, | <0.001 | 222 | −0.98 | −1.38, | <0.001 |
| 375 | | | −0.47 | | | | −0.38 | | | | −0.57 | |
| OPN-375 | 40 | −1.11 | −1.91, | 0.006 | 71 | −0.95 | −1.53, | 0.001 | 110 | −1.01 | −1.47, | <0.001 |
| 372 μg | | | −0.32 | | | | −0.38 | | | | −0.54 | |
| OPN375 | 40 | −1.21 | −2.01, | 0.003 | 72 | −0.80 | −1.37, | 0.007 | 112 | −0.95 | −1.42, | <0.001 |
| 186 μg | | | −0.42 | | | | −0.23 | | | | −0.48 | |
| Placebo | 40 | — | — | — | 72 | — | — | — | 112 | — | — | — |

Average of Percentages of Opacified Volume in Ethmoid and Maxillary
Sinuses - Change from Baseline to Week 24/Early Termination
Full Analysis Set

| | | | | | | | | | | | | |
|---|---|---|---|---|---|---|---|---|---|---|---|---|
| All OPN- | 202 | −5.24 | −8.78, | 0.004 | 139 | −7.52 | −11.82, | <0.001 | 341 | −6.20 | −8.92, | <0.001 |
| 375 | | | −1.70 | | | | −3.22 | | | | −3.47 | |
| OPN-375 | 101 | −6.45 | −10.53, | 0.002 | 69 | −5.82 | −10.81, | 0.022 | 170 | −6.2.3 | −9.39, | <0.001 |
| 372 μg | | | −2.38 | | | | −0.83 | | | | −3.08 | |
| OPN-375 | 101 | −4.03 | −8.15, | 0.055 | 70 | −9.22 | −14.15, | <0.001 | 171 | −6.16 | −9.31, | <0.001 |
| 186 μg | | | 0.09 | | | | −4.29 | | | | −3.01 | |
| Placebo | 100 | — | — | — | 67 | — | — | — | 167 | — | — | — |

CS Patients Without Nasal Polyps

| | | | | | | | | | | | | |
|---|---|---|---|---|---|---|---|---|---|---|---|---|
| All OPN- | 74 | −2.00 | −7.84, | 0.502 | 139 | −7.52 | −11.82, | <0.001 | 213 | −5.67 | −9.13, | 0.001 |
| 375 | | | 3.84 | | | | −3.22 | | | | −2.21 | |
| OPN-375 | 38 | −5.56 | −12.23, | 0.102 | 69 | −5.82 | −10.81, | 0.002 | 107 | −5.88 | −9.89, | 0.004 |
| 372 μg | | | 1.11 | | | | −0.83 | | | | −1.87 | |
| OPN-375 | 36 | 1.56 | −5.29, | 0.655 | 70 | −9.22 | −14.15, | <0.001 | 106 | −5.46 | −9.47, | 0.008 |
| 186 μg | | | 8.41 | | | | −4.29 | | | | −1.44 | |
| Placebo | 38 | — | — | — | 67 | — | — | — | 105 | — | — | — |

Abbreviations: CI = confidence interval; CRSsNP = chronic rhinosinusitis without nasal polyps; CS = chronic sinusitis; FAS = Full Analysis Set; LSMD = least squares mean treatment difference

TABLE 53
Co-Primary Endpoint Analysis Treatment Difference
Comparisons by Population (Supplementary Estimand 2).

| | | | | Study B (FAS = | | | | | | | |
|---|---|---|---|---|---|---|---|---|---|---|---|
| Study A | | | | CRSsNP) | | | | Pooled Data | | | |
| | | 95% | P- | | | 95% | P- | | | 95% | P- |
| n | LSMD | CI | value | n | LSMD | CI | value | n | LSMD | CI | value |

Composite Symptom Score - Change from Baseline to End of Week 4
Full Analysis Set

| | | | | | | | | | | | | |
|---|---|---|---|---|---|---|---|---|---|---|---|---|
| All OPN- | 215 | −0.95 | −1.32, | <0.001 | 143 | −0.81 | −1.30, | 0.001 | 358 | −0.90 | −1.19, | <0.001 |
| 375 | | | −0.59 | | | | −0.33 | | | | −0.61 | |
| OPN-375 | 106 | −0.97 | −1.39, | <0.001 | 71 | −0.91 | −1.47, | 0.001 | 177 | −0.95 | −1.28, | <0.001 |
| 372 μg | | | −0.55 | | | | −0.36 | | | | −0.61 | |
| OPN-375 | 109 | −0.94 | −1.36, | <0.001 | 72 | −0.71 | −1.27, | 0.013 | 181 | −0.85 | −1.18, | <0.001 |
| 186 μg | | | −0.52 | | | | −0.15 | | | | −0.51 | |
| Placebo | 108 | — | — | — | 73 | — | — | — | 181 | — | — | — |

CS Patients Without Nasal Polyps

| | | | | | | | | | | | | |
|---|---|---|---|---|---|---|---|---|---|---|---|---|
| All OPN- | 81 | −0.92 | −1.51, | 0.003 | 143 | −0.81 | −1.30, | 0.001 | 223 | −0.84 | −1.21, | <0.001 |
| 375 | | | −0.32 | | | | −0.33 | | | | −0.47 | |
| OPN-375 | 40 | −0.91 | −1.60, | 0.010 | 71 | −0.91 | −1.47, | 0.001 | 110 | −0.90 | −1.33, | <0.001 |
| 372 μg | | | −0.22 | | | | −0.36 | | | | −0.47 | |
| OPN375 | 41 | −0.93 | −1.61, | 0.008 | 72 | −0.71 | −1.27, | 0.013 | 113 | −0.78 | −1.21, | <0.001 |
| 186 μg | | | −0.24 | | | | −0.15 | | | | −0.36 | |
| Placebo | 41 | — | — | — | 73 | — | — | — | 114 | — | — | — |

Average of Percentages of Opacified Volume in Ethmoid and Maxillary
Sinuses - Change from Baseline to Week 24/Early Termination
Full Analysis Set

| | | | | | | | | | | | | |
|---|---|---|---|---|---|---|---|---|---|---|---|---|
| All OPN- | 207 | −4.23 | −7.62 | 0.014 | 140 | −7.15 | −11.17, | <0.001 | 347 | −5.45 | −8.03, | <0.001 |
| 375 | | | −0.85 | | | | −3.12 | | | | −2.86 | |
| OPN-375 | 105 | −4.00 | −7.89, | 0.044 | 70 | −6.36 | −11.01, | 0.007 | 175 | −4.99 | −7.96, | 0.001 |
| 372 μg | | | −0.11 | | | | −1.72 | | | | −2.01 | |
| OPN-375 | 102 | −4.47 | −8.42, | 0.027 | 70 | −7.93 | −12.59, | <0.001 | 172 | −5.91 | −8.92, | <0.001 |
| 186 μg | | | −0.52 | | | | −3.27 | | | | −2.89 | |
| Placebo | 104 | — | — | — | 67 | — | — | — | 171 | — | — | — |

CS Patients Without Nasal Polyps

| | | | | | | | | | | | | |
|---|---|---|---|---|---|---|---|---|---|---|---|---|
| All OPN- | 77 | −1.81 | (−7.27, | 0.517 | 140 | −7.15 | −11.17, | <0.001 | 217 | −5.35 | −8.62, | 0.001 |
| 375 | | | 3.66) | | | | −3.12 | | | | −2.08 | |
| OPN-375 | 39 | −3.16 | (−9.50, | 0.329 | 70 | −6.36 | −11.01, | 0.007 | 109 | −5.35 | −9.14, | 0.006 |
| 372 μg | | | 3.18) | | | | −1.72 | | | | −1.56 | |
| OPN-375 | 38 | −0.45 | (−6.81, | 0.889 | 70 | −7.93 | −12.59, | <0.001 | 108 | −5.35 | −9.15, | 0.006 |
| 186 μg | | | 5.90) | | | | −3.27 | | | | −1.56 | |
| Placebo | 41 | — | — | — | 67 | — | — | — | 108 | — | — | — |

Abbreviations: CI = confidence interval; CRSsNP = chronic rhinosinusitis without nasal polyps; CS = chronic sinusitis; LSMD = least squares mean treatment difference; FAS = Full Analysis Set.

TABLE 54
Key Secondary Endpoint Analysis Treatment Difference Comparisons
for Change from Baseline to End of Week 4 in Instantaneous
Composite Symptom Score for Baseline Intranasal Steroid Population.

| | | | | Alternative primary | | | | | | | |
|---|---|---|---|---|---|---|---|---|---|---|---|
| | | | | estimand - Intercurrent | | | | | | | |
| | | | | Events Include | | | | | | | |
| Primary Estimand | | | | Antibiotics for AECRS | | | | Supplementary Estimand 2 | | | |
| | | 95% | P- | | | 95% | P- | | | 95% | P- |
| n | LSMD | CI | value | n | LSMD | CI | value | n | LSMD | CI | value |

Full Analysis Set

| | | | | | | | | | | | | |
|---|---|---|---|---|---|---|---|---|---|---|---|---|
| All OPN- | 169 | −0.70 | −1.09, | <0.001 | 169 | −1.03 | −1.46, | <0.001 | 169 | −0.66 | −1.04, | <0.001 |
| 375 | | | −0.30 | | | | −0.60 | | | | −0.28 | |
| OPN-375 | 83 | −0.63 | −1.10, | 0.009 | 83 | −0.95 | −1.46, | <0.001 | 83 | −0.62 | −1.07, | 0.007 |
| 372 μg | | | −0.16 | | | | −0.44 | | | | −0.17 | |
| OPN-375 | 86 | −0.76 | −1.23, | 0.001 | 86 | −1.11 | −1.62, | <0.001 | 86 | −0.69 | −1.14, | 0.002 |
| 186 μg | | | −0.30 | | | | −0.60 | | | | −0.25 | |
| Placebo | 104 | — | — | — | 104 | — | — | — | 105 | — | — | — |

CS Patients Without Nasal Polyps

| | | | | | | | | | | | | |
|---|---|---|---|---|---|---|---|---|---|---|---|---|
| All OPN- | 97 | −0.54 | −1.06, | 0.045 | 97 | −0.88 | −1.45, | 0.003 | 97 | −0.58 | −1.08, | 0.023 |
| 375 | | | −0.01 | | | | −0.30 | | | | −0.08 | |
| OPN-375 | 48 | −0.45 | −1.07, | 0.150 | 48 | −0.77 | −1.44, | 0.025 | 48 | −0.55 | −1.14, | 0.068 |
| 372 μg | | | 0.16 | | | | −0.10 | | | | 0.04 | |
| OPN375 | 49 | −0.62 | −1.24, | 0.050 | 49 | −0.98 | −1.65, | 0.005 | 49 | −0.61 | −1.21, | 0.042 |
| 186 μg | | | −0.00 | | | | −0.31 | | | | −0.02 | |
| Placebo | 60 | — | — | — | 60 | — | — | — | 62 | — | — | — |

Abbreviations: AECRS = acute exacerbations for chronic rhinosinusitis; CI = confidence interval; CRS = chronic rhinosinusitis; CS = chronic sinusitis; LSMD = least squares mean treatment difference

TABLE 55
Change from Baseline to Week 24/ET in SNOT-22 Total Score (Primary Estimand).

| Study A | | | | Study B (FAS = CRSsNP) | | | | Pooled Data | | | |
|---|---|---|---|---|---|---|---|---|---|---|---|
| | | 95% | P- | | | 95% | P- | | | 95% | P- |
| n | LSMD | CI | value | n | LSMD | CI | value | n | LSMD | CI | value |

Full Analysis Set

| | | | | | | | | | | | | |
|---|---|---|---|---|---|---|---|---|---|---|---|---|
| All OPN- | 194 | −10.25 | −14.32, | <0.001 | 138 | −8.77 | −14.03, | 0.001 | 332 | −9.62 | −12.86, | <0.001 |
| 375 | | | −6.17 | | | | −3.50 | | | | −6.38 | |
| OPN-375 | 96 | −12.61 | −17.32, | <0.001 | 68 | −6.81 | −12.92, | 0.029 | 164 | −10.19 | −13.93, | <0.001 |
| 372 | | | −7.90 | | | | −0.71 | | | | −6.44 | |
| OPN- | 98 | −7.89 | −12.57, | 0.001 | 70 | −10.72 | −16.79, | <0.001 | 168 | −9.06 | −12.78, | <0.001 |
| 375 186 | | | −3.21 | | | | −4.65 | | | | −5.33 | |
| Placebo | 93 | — | — | — | 70 | — | — | — | 163 | — | — | — |

CS Patients Without Nasal Polyps

| | | | | | | | | | | | | |
|---|---|---|---|---|---|---|---|---|---|---|---|---|
| All OPN- | 73 | −2.67 | −9.32, | 0.429 | 138 | −8.77 | −14.03, | 0.001 | 211 | −6.73 | −10.78, | 0.001 |
| 375 | | | 3.97 | | | | −3.50 | | | | −2.68 | |
| OPN-375 | 37 | −5.81 | −13.46, | 0.136 | 68 | −6.81 | −12.92, | 0.029 | 105 | −6.52 | −11.20, | 0.006 |
| 372 | | | 1.84 | | | | −0.71 | | | | −1.84 | |
| OPN-375 | 36 | 0.46 | −7.17, | 0.905 | 70 | −10.72 | −16.79, | <0.001 | 106 | −6.95 | −11.62, | 0.004 |
| 186 | | | 8.10 | | | | −4.65 | | | | −2.28 | |
| Placebo | 35 | — | — | — | 70 | — | — | — | 105 | — | — | — |

Abbreviations: CI = confidence interval; CRSsNP = chronic rhinosinusitis without nasal polyps; CS = chronic sinusitis; ET = early termination; FAS = Full Analysis Set; LSMD = least squares mean treatment difference; SNOT-22 = Sinonasal Outcome Test 22-item version

TABLE 56
Change from Baseline to Week 24/ET in SNOT-22
Total Score (Alternative primary estimand).

| | | | | Study B (FAS = | | | | | | | |
|---|---|---|---|---|---|---|---|---|---|---|---|
| Study A | | | | CRSsNP) | | | | Pooled Data | | | |
| | | 95% | P- | | | 95% | P- | | | 95% | P- |
| n | LSMD | CI | value | n | LSMD | CI | value | n | LSMD | CI | value |

Full Analysis Set

| | | | | | | | | | | | | |
|---|---|---|---|---|---|---|---|---|---|---|---|---|
| All OPN- | 196 | −11.71 | −16.60, | <0.001 | 138 | −9.27 | −14.83, | 0.001 | 334 | −10.65 | −14.34, | <0.001 |
| 375 | | | −6.82 | | | | −3.70 | | | | −6.96 | |
| OPN-375 | 97 | −15.12 | −20.77, | <0.001 | 68 | −6.89 | −13.34, | 0.036 | 165 | −11.64 | −15.90, | <0.001 |
| 372 | | | −9.47 | | | | −0.44 | | | | 7.37 | |
| OPN- | 99 | −8.30 | −13.92, | 0.004 | 70 | −11.64 | −18.06, | <0.001 | 169 | −9.66 | −13.91, | <0.001 |
| 375 186 | | | −2.69 | | | | −5.23 | | | | −5.42 | |
| Placebo | 96 | — | — | — | 70 | — | — | — | 166 | — | — | — |

CS Patients Without Nasal Polyps

| | | | | | | | | | | | | |
|---|---|---|---|---|---|---|---|---|---|---|---|---|
| All OPN- | 73 | 6.10 | −14.10, | 0.135 | 138 | −9.27 | −14.83, | 0.001 | 211 | −8.16 | −12.79, | <0.001 |
| 375 | | | 1.91 | | | | −3.70 | | | | −3.53 | |
| OPN-375 | 37 | −10.42 | −19.65, | 0.027 | 68 | −6.89 | −13.34, | 0.036 | 105 | −8.01 | −13.36, | 0.003 |
| 372 | | | −1.18 | | | | −0.44 | | | | −2.66 | |
| OPN-375 | 36 | −1.78 | −11.00, | 0.704 | 70 | −11.64 | −18.06, | <0.001 | 106 | −8.31 | −13.65, | 0.002 |
| 186 | | | 7.44 | | | | −5.23 | | | | −2.96 | |
| Placebo | 36 | — | — | — | 70 | — | — | — | 106 | — | — | — |

Abbreviations: CI = confidence interval; CRSsNP = chronic rhinosinusitis without nasal polyps; CS = chronic sinusitis; ET = early termination; FAS = Full Analysis Set; LSMD = least squares mean treatment difference; SNOT-22 = Sinonasal Outcome Test 22-item version

TABLE 57
Change from Baseline to Week 24/ET in Pittsburgh Sleep Quality Index Global Score.

| Study A | | | | Study B (FAS = CRSsNP) | | | | Pooled Data | | | |
|---|---|---|---|---|---|---|---|---|---|---|---|
| | | 95% | P- | | | 95% | P- | | | 95% | P- |
| n | LSMD | CI | value | n | LSMD | CI | value | n | LSMD | CI | value |

Full Analysis Set

| | | | | | | | | | | | | |
|---|---|---|---|---|---|---|---|---|---|---|---|---|
| All OPN- | 196 | −0.35 | (−1.06, | 0.328 | 137 | −1.22 | (−1.92, | <0.001 | 333 | −0.71 | (−1.22, | 0.006 |
| 375 | | | 0.36) | | | | −0.51) | | | | −0.21) | |
| OPN-375 | 97 | −0.68 | (−1.49, | 0.103 | 69 | −0.75 | (−1.57, | 0.068 | 166 | −0.71 | (−1.30, | 0.017 |
| 372 μg | | | 0.14) | | | | 0.06) | | | | −0.13) | |
| OPN-375 | 99 | −0.03 | (−0.84, | 0.946 | 68 | −1.68 | (−2.50, | <0.001 | 167 | −0.71 | (−1.30, | 0.017 |
| 186 μg | | | 0.79) | | | | −0.86) | | | | −0.13) | |
| Placebo | 92 | — | — | — | 69 | — | — | — | 161 | — | — | — |

CS Patients Without Nasal Polyps

| | | | | | | | | | | | | |
|---|---|---|---|---|---|---|---|---|---|---|---|---|
| All OPN- | ND | ND | ND | ND | 137 | −1.22 | (−1.92, | <0.001 | 211 | −0.74 | (−1.38, | 0.022 |
| 375 | | | | | | | −0.51) | | | | −0.11) | |
| OPN-375 | ND | ND | ND | ND | 69 | −0.75 | (−1.57, | 0.068 | 107 | −0.51 | (−1.25, | 0.168 |
| 372 μg | | | | | | | 0.06) | | | | 0.22) | |
| OPN-375 | ND | ND | ND | ND | 68 | −1.68 | (−2.50, | <0.001 | 104 | −0.97 | (−1.71, | 0.010 |
| 186 μg | | | | | | | −0.86) | | | | −0.24) | |
| Placebo | ND | ND | ND | ND | 69 | — | — | — | 103 | — | — | — |

Abbreviations: CI = confidence interval; CRSsNP = chronic rhinosinusitis without nasal polyps; ET = early termination; FAS = Full Analysis Set; LSMD = least squares mean treatment difference; ND = not determined

TABLE 58
Week 4 and Week 24/ET Patient Global Impression of Change Score.

| | | | | Study B (FAS = | | | | | | | |
|---|---|---|---|---|---|---|---|---|---|---|---|
| Study A | | | | CRSsNP) | | | | Pooled Data | | | |
| | | 95% | P- | | | 95% | P- | | | 95% | P- |
| n | OR | CI | value | n | OR | CI | value | n | OR | CI | value |

WEEK 4
Full Analysis Set - Improved

| | | | | | | | | | | | | |
|---|---|---|---|---|---|---|---|---|---|---|---|---|
| All OPN-375 | 212 | 2.65 | (1.60, | <0.001 | 142 | 2.55 | (1.39, | 0.003 | 354 | 2.60 | (1.77, | <0.001 |
| | | | 4.38) | | | | 4.67) | | | | 3.81) | |
| OPN-375 | 103 | 2.50 | (1.38, | 0.003 | 71 | 3.52 | (1.67, | 0.001 | 174 | 2.87 | (1.80, | <0.001 |
| 372 μg | | | 4.54) | | | | 7.41) | | | | 4.55) | |
| OPN-375 | 109 | 2.80 | (1.54, | <0.001 | 71 | 1.85 | (0.93, | 0.078 | 180 | 2.35 | (1.51, | <0.001 |
| 186 μg | | | 5.09) | | | | 3.66) | | | | 3.67) | |
| Placebo | 104 | — | — | — | 72 | — | — | — | 176 | — | — | — |

Full Analysis Set - Much Improved

| | | | | | | | | | | | | |
|---|---|---|---|---|---|---|---|---|---|---|---|---|
| All OPN-375 | 212 | 7.92 | (3.54, | <0.001 | 142 | 5.22 | (2.28, | <0.001 | 354 | 6.47 | (3.65, | <0.001 |
| | | | 17.75) | | | | 11.95) | | | | 11.47) | |
| OPN-375 | 103 | 8.65 | (3.68, | <0.001 | 71 | 5.20 | (2.12, | <0.001 | 174 | 6.80 | (3.68, | <0.001 |
| 372 μg | | | 20.31) | | | | 12.75) | | | | 12.55) | |
| OPN-375 | 109 | 7.26 | (3.10, | <0.001 | 71 | 5.24 | (2.14, | <0.001 | 180 | 6.15 | (3.34, | <0.001 |
| 186 μg | | | 17.03) | | | | 12.85) | | | | 11.35) | |
| Placebo | 104 | — | — | — | 72 | — | — | — | 176 | — | — | — |

CS Patients Without Nasal Polyps - Improved

| | | | | | | | | | | | | |
|---|---|---|---|---|---|---|---|---|---|---|---|---|
| All OPN-375 | ND | ND | ND | ND | 142 | 2.55 | (1.39, | 0.003 | 221 | 2.37 | (1.46, | <0.001 |
| | | | | | | | 4.67) | | | | 3.84) | |
| OPN-375 | ND | ND | ND | ND | 71 | 3.52 | (1.67, | 0.001 | 110 | 3.03 | (1.68, | <0.001 |
| 372 μg | | | | | | | 7.41) | | | | 5.45) | |
| OPN-375 | ND | ND | ND | ND | 71 | 1.85 | (0.93, | 0.078 | 111 | 1.85 | (1.07, | 0.028 |
| 186 μg | | | | | | | 3.66) | | | | 3.21) | |
| Placebo | ND | — | — | — | 72 | — | — | — | 111 | — | — | — |

CS Patients Without Nasal Polyps - Much Improved

| | | | | | | | | | | | | |
|---|---|---|---|---|---|---|---|---|---|---|---|---|
| All OPN-375 | ND | ND | ND | ND | 142 | 5.22 | (2.28, | <0.001 | 221 | 5.19 | (2.67, | <0.001 |
| | | | | | | | 11.95) | | | | 10.07) | |
| OPN-375 | ND | ND | ND | ND | 71 | 5.20 | (2.12, | <0.001 | 110 | 5.76 | (2.82, | <0.001 |
| 372 μg | | | | | | | 12.75) | | | | 11.78) | |
| OPN-375 | ND | ND | ND | ND | 71 | 5.24 | (2.14, | <0.001 | 111 | 4.67 | (2.28, | <0.001 |
| 186 μg | | | | | | | 12.85) | | | | 9.60) | |
| Placebo | ND | — | — | — | 72 | — | — | — | 111 | — | — | — |

WEEK 24
Full Analysis Set - Improved

| | | | | | | | | | | | | |
|---|---|---|---|---|---|---|---|---|---|---|---|---|
| All OPN-375 | 197 | 2.85 | (1.64, | <0.001 | 139 | 4.78 | (2.31, | <0.001 | 336 | 3.40 | (2.21, | <0.001 |
| | | | 4.93) | | | | 9.91) | | | | 5.21) | |
| OPN-375 | 98 | 3.10 | (1.59, | <0.001 | 69 | 6.81 | (2.54, | <0.001 | 167 | 4.01 | (2.34, | <0.001 |
| 372 μg | | | 6.04) | | | | 18.27) | | | | 6.85) | |
| OPN-375 | 99 | 2.61 | (1.37, | 0.004 | 70 | 3.36 | (1.49, | 0.004 | 169 | 2.88 | (1.75, | <0.001 |
| 186 μg | | | 4.97) | | | | 7.60) | | | | 4.74) | |
| Placebo | 94 | — | — | — | 69 | — | — | — | 163 | — | — | — |

Full Analysis Set - Much Improved

| | | | | | | | | | | | | |
|---|---|---|---|---|---|---|---|---|---|---|---|---|
| All OPN-375 | 197 | 2.88 | (1.66, | <0.001 | 139 | 4.59 | (2.39, | <0.001 | 336 | 3.51 | (2.31, | <0.001 |
| | | | 5.00) | | | | 8.83) | | | | 5.33) | |
| OPN-375 | 98 | 3.49 | (1.87, | <0.001 | 69 | 4.05 | (1.94, | <0.001 | 167 | 3.72 | (2.32, | <0.001 |
| 372 μg | | | 6.49) | | | | 8.45) | | | | 5.97) | |
| OPN-375 | 99 | 2.38 | (1.28, | 0.006 | 70 | 5.21 | (2.48, | <0.001 | 169 | 3.30 | (2.06, | <0.001 |
| 186 μg | | | 4.42) | | | | 10.92) | | | | 5.28) | |
| Placebo | 94 | — | — | — | 69 | — | — | — | 163 | — | — | — |

CS Patients Without Nasal Polyps - Improved

| All OPN-375 | ND | ND | ND | ND | 139 | 4.78 | (2.31, | <0.001 | 213 | 2.86 | (1.65, | <0.001 |
|---|---|---|---|---|---|---|---|---|---|---|---|---|
| | | | | | | | 9.91) | | | | 4.94) | |
| OPN-375 | ND | ND | ND | ND | 69 | 6.81 | (2.54, | <0.001 | 107 | 3.42 | (1.73, | <0.001 |
| 372 μg | | | | | | | 18.27) | | | | 6.78) | |
| OPN-375 | ND | ND | ND | ND | 70 | 3.36 | (1.49, | 0.004 | 106 | 2.39 | (1.27, | 0.007 |
| 186 μg | | | | | | | 7.60) | | | | 4.51) | |
| Placebo | ND | — | — | — | 69 | — | — | — | 105 | — | — | — |
| CS Patients Without Nasal Polyps - Much Improved | | | | | | | | | | | | |
| All OPN-375 | ND | ND | ND | ND | 139 | 4.59 | (2.39, | <0.001 | 213 | 3.49 | (2.09, | <0.001 |
| | | | | | | | 8.83) | | | | 5.86) | |
| OPN-375 | ND | ND | ND | ND | 69 | 4.05 | (1.94, | <0.001 | 107 | 3.45 | (1.93, | <0.001 |
| 372 μg | | | | | | | 8.45) | | | | 6.18) | |
| OPN-375 | ND | ND | ND | ND | 70 | 5.21 | (2.48, | <0.001 | 106 | 3.54 | (1.97, | <0.001 |
| 186 μg | | | | | | | 10.92) | | | | 6.35) | |
| Placebo | ND | — | — | — | 69 | — | — | — | 105 | — | — | — |

Abbreviations: CI = confidence interval; CRSsNP = chronic rhinosinusitis without nasal polyps; CS = chronic sinusitis; ET = early termination; FAS = Full Analysis Set; ND = not determined; OR = odds ratio

Averse Events

In placebo-controlled studies in chronic sinusitis, the overall incidence of TEAEs as well as treatment-related TEAEs was comparable between the combined active treatment and placebo groups (48.0% vs 47.6% and 9.8% vs 7.0%, respectively). No subjects died. The incidence of severe TEAEs, other SAEs, and AEs leading to discontinuation was low (≤3%), and similar in all treatment groups.

In placebo-controlled studies in chronic sinusitis, epistaxis was the most commonly reported (≥3%) TEAE in OPN-375-treated subjects, with a higher incidence of this event reported at the higher dose than the lower dose (Table 59). No other TEAE had a comparable dose-related pattern, and no other common TEAE had an incidence that was higher in both active dose groups than placebo (i.e., the incidence in the placebo group was intermediate between the 2 active dose groups).

TABLE 59
Summary of TEAEs Reported in ³3% of Subjects in Any Group or the Combined
OPN-375 Group by System Organ Class and Preferred Term: Placebo-controlled
Studies in Chronic Sinusitis (Safety Analysis Set).

| | | | OPN-375 | | | | | | | | |
|---|---|---|---|---|---|---|---|---|---|---|---|
| | | | | | | OPN-375 | | | | | |
| | | | OPN-375 186 μg | | | 372 μg | | | All OPN-375 | | |
| System Organ | | | (N = 184) | | | (N = 183) | | | (N = 367) | | |
| Class | Placebo | | | | 95% | | | 95% | | | 95% |
| MedDRA | (N = 187) | | | | CI vs | | | CI vs | | | CI vs |
| Preferred Term | N (%) | | N (%) | | Placebo | N (%) | | Placebo | N (%) | | Placebo |
| Subjects with at | 89 | (47.6) | 81 | (44.0) | ND | 95 | (51.9) | ND | 176 | (48.0) | ND |
| least 1 AE | | | | | | | | | | | |
| Eye disorders | 8 | (4.3) | 12 | (6.5) | ND | 10 | (5.5) | ND | 22 | (6.0) | ND |
| Cataract cortical | 2 | (1.1) | 6 | (3.3) | (−1.0, 6.0) | 3 | (1.6) | ND | 9 | (2.5) | (−1.5, 3.7) |
| Infections and | 57 | (30.5) | 48 | (26.1) | ND | 41 | (22.4) | ND | 89 | (24.3) | ND |
| infestations | | | | | | | | | | | |
| COVID-19 | 8 | (4.3) | 5 | (2.7) | (−5.8, 2.5) | 12 | (6.6) | (−2.5, 7.4) | 17 | (4.6) | (−3.9, 3.8) |
| Nasopharyngitis | 8 | (4.3) | 9 | (4.9) | (−4.0, 5.3) | 7 | (3.8) | (−4.9, 4.0) | 16 | (4.4) | (−4.2, 3.5) |
| Sinusitis | 12 | (6.4) | 7 | (3.8) | (−7.6, 2.0) | 8 | (4.4) | (−7.1, 2.8) | 15 | (4.1) | (−7.1, 1.4) |
| Chronic | 12 | (6.4) | 6 | (3.3) | (−8.0, 1.3) | 4 | (2.2) | (−9.0, −0.1) | 10 | (2.7) | (−8.4, −0.2) |
| sinusitis^{a} | | | | | | | | | | | |
| Nervous system | 11 | (5.9) | 11 | (6.0) | ND | 18 | (9.8) | ND | 29 | (7.9) | ND |
| disorders | | | | | | | | | | | |
| Headache | 7 | (3.7) | 4 | (2.2) | (−5.6, 2.2) | 10 | (5.5) | (−2.8, 6.5) | 14 | (3.8) | (−4.0, 3.2) |
| Respiratory, | 27 | (14.4) | 27 | (14.7) | ND | 35 | (19.1) | ND | 62 | (16.9) | ND |
| thoracic and | | | | | | | | | | | |
| mediastinal | | | | | | | | | | | |
| disorders | | | | | | | | | | | |
| Epistaxis | 1 | (0.5) | 9 | (4.9) | (1.3, 8.6) | 20 | (10.9) | (6.3, 15.8) | 29 | (7.9) | (4.3, 10.7) |
| Asthma | 5 | (2.7) | 5 | (2.7) | (−3.7, 3.9) | 6 | (3.3) | (−3.3, 4.6) | 11 | (3.0) | (−3.3, 3.1) |
| Nasal polyps | 12 | (6.4) | 4 | (2.2) | (−9.0, −0.1) | 2 | (1.1) | (−9.9, −1.7) | 6 | (1.6) | (−9.4, −1.6) |

AE = adverse event; CI = confidence interval; COVID-19 = coronavirus disease 2019; CRS = chronic rhinosinusitis; MedDRA = Medical Dictionary for Regulatory Activities; ND = not determined; TEAE = treatment-emergent adverse event.
Note:
Preferred terms are sorted by descending order of incidence within system organ class for the all OPN-375 group. Subjects are counted only once in each preferred term category, and only once in each system organ class category.
Note:
The 95% CI is based on the preferred term risk difference (Active - Placebo). CIs were only produced if at least one treatment group had at least 4 subjects with the preferred term AE.
^{a}All AEs that coded to the MedDRA preferred term of ″chronic sinusitis″ experienced a CRS exacerbation.

Of those AEs that were both ≥3%, and greater than placebo, epistaxis, headache, and nasopharyngitis could be considered related to treatment with OPN-375 and are therefore treated as adverse reactions (Table 60).

TABLE 60
Summary of Adverse Reactions Reported in ≥3% in Subjects on
Active Treatment and Greater Than Placebo.

| | EDS- | OPN-375 | OPN-375 | |
|---|---|---|---|---|
| | Placebo | 186 μg | 372 μg | Total |
| | (N = 187) | (N = 184) | (N = 183) | OPN-375 |
| Adverse Reaction | n (%) | n (%) | n (%) | (N = 367) |
| Epistaxis | 1 (0.5) | 9 (4.9) | 20 (10.9) | 29 (7.9) |
| Headache | 7 (3.7) | 4 (2.2) | 10 (5.5) | 14 (3.8) |
| Nasopharyngitis | 8 (4.3) | 9 (4.9) | 7 (3.8) | 16 (4.4) |

EDS = exhalation delivery system

The overall incidence of TEAEs was comparable between the OPN-375 (42.1% in the 186 μg group and 54.0% in the 372 mg group) and placebo (44.1%) groups for the cohort of CS subjects who did not have polyps in the nasal cavity at baseline (Table 61). “Epistaxis” was the most commonly reported preferred term (PT) in OPN-375-treated subjects, and the incidence of this TEAE was higher in patients treated with OPN-375 (incidence of 0.0%, 6.1%, and 8.8% in the placebo, 186 μg OPN-375, and 372 μg OPN-375 treatment groups, respectively).

TABLE 61
Summary of TEAEs Reported in ³3 Subjects in Any Group or the Combined
OPN-375 Group by System Organ Class and Preferred Term: Placebo-controlled Studies
in Chronic Sinusitis (Integrated Data, Phenotype without NP at Baseline)

| | | | OPN-375 | | | | | | | | |
|---|---|---|---|---|---|---|---|---|---|---|---|
| | | | 186 μg | | | 372 μg | | | Total | | |
| | | | (N = 114) | | | (N = 113) | | | (N = 227) | | |
| System Organ Class | Placebo | | | | 95% CI | | | 95% | | | 95% |
| MedDRA Preferred | (N = 118) | | | | vs | | | CI vs | | | CI vs |
| Term | N (%) | | N (%) | | Placebo | N (%) | | Placebo | N (%) | | Placebo |
| Subjects with at least 1 | 52 | (44.1) | 48 | (42.1) | ND | 61 | (54.0) | ND | 109 | (48.0) | ND |
| AE | | | | | | | | | | | |
| Eye disorders | 6 | (5.1) | 4 | (3.5) | ND | 4 | (3.5) | ND | 8 | (3.5) | ND |
| Cataract nuclear | 2 | (1.7) | 1 | (0.9) | ND | 2 | (1.8) | ND | 3 | (1.3) | ND |
| Gastrointestinal | 2 | (1.7) | 4 | (3.5) | ND | 6 | (5.3) | ND | 10 | (4.4) | ND |
| disorders | | | | | | | | | | | |
| Nausea | 0 | | 2 | (1.8) | ND | 1 | (0.9) | ND | 3 | (1.3) | ND |
| Infections and | 37 | (31.4) | 30 | (26.3) | ND | 28 | (24.8) | ND | 58 | (25.6) | ND |
| infestations | | | | | | | | | | | |
| COVID-19 | 5 | (4.2) | 4 | (3.5) | (−6.5, 5.0) | 10 | (8.8) | (−1.9, 11.9) | 14 | (6.2) | (−3.9, 6.6) |
| Nasopharyngitis | 8 | (6.8) | 5 | (4.4) | (−9.0, 4.0) | 5 | (4.4) | (−9.0, 4.1) | 10 | (4.4) | (−8.8, 2.5) |
| Chronic sinusitis | 7 | (5.9) | 5 | (4.4) | (−8.0, 4.7) | 4 | (3.5) | (−8.7, 3.6) | 9 | (4.0) | (−8.1, 2.6) |
| Sinusitis | 9 | (7.6) | 4 | (3.5) | (−10.8, 2.1) | 5 | (4.4) | (−10.1, 3.3) | 9 | (4.0) | (−10.2, 1.2) |
| Influenza | 0 | | 5 | (4.4) | (1.1, 9.9) | 0 | | ND | 5 | (2.2) | (−1.0, 5.1) |
| Upper respiratory | 2 | (1.7) | 4 | (3.5) | (−2.9, 7.2) | 1 | (0.9) | ND | 5 | (2.2) | (−3.9, 3.7) |
| tract infection | | | | | | | | | | | |
| Acute sinusitis | 1 | (0.8) | 2 | (1.8) | ND | 1 | (0.9) | ND | 3 | (1.3) | ND |
| Investigations | 2 | (1.7) | 3 | (2.6) | ND | 2 | (1.8) | ND | 5 | (2.2) | ND |
| Intraocular pressure | 0 | | 2 | (1.8) | ND | 1 | (0.9) | ND | 3 | (1.3) | ND |
| increased | | | | | | | | | | | |
| Nervous system | 9 | (7.6) | 7 | (6.1) | ND | 12 | (10.6) | ND | 19 | (8.4) | ND |
| disorders | | | | | | | | | | | |
| Headache | 6 | (5.1) | 3 | (2.6) | (−8.4, 3.0) | 8 | (7.1) | (−4.6, 8.9) | 11 | (4.8) | (−6.2, 4.4) |
| Psychiatric disorders | 3 | (2.5) | 2 | (1.8) | ND | 4 | (3.5) | ND | 6 | (2.6) | ND |
| Depression | 1 | (0.8) | 0 | | ND | 3 | (2.7) | ND | 3 | (1.3) | ND |
| Respiratory, thoracic | 16 | (13.6) | 15 | (13.2) | ND | 17 | (15.0) | ND | 32 | (14.1) | ND |
| and mediastinal | | | | | | | | | | | |
| disorders | | | | | | | | | | | |
| Epistaxis | 0 | | 7 | (6.1) | (2.9, 12.2) | 10 | (8.8) | (4.9, 15.5) | 17 | (7.5) | (4.2, 11.7) |
| Asthma | 5 | (4.2) | 3 | (2.6) | (−7.3, 3.8) | 3 | (2.7) | (−7.3, 3.8) | 6 | (2.6) | (−7.1, 2.3) |
| Nasal polyps | 8 | (6.8) | 3 | (2.6) | (−10.5, 1.6) | 1 | (0.9) | (−12.1, −1.1) | 4 | (1.8) | (−11.2, 0.9) |

| | | | | | | | | | | |
|---|---|---|---|---|---|---|---|---|---|---|
| Vascular disorders | 0 | 2 | (1.8) | ND | 2 | (1.8) | ND | 4 | (1.8) | ND |
| Hypertension | 0 | 1 | (0.9) | ND | 2 | (1.8) | ND | 3 | (1.3) | ND |

Abbreviations: AE = adverse event; CI = confidence interval; COVID-19 = coronavirus disease 2019; MedDRA = Medical Dictionary for Regulatory Activities; ND = not determined
Notes:
Preferred terms are sorted by descending order of incidence within system organ class for OPN-375 Total. Subjects are counted only once in each preferred term category, and only once in each system organ class category.
Note:
The 95% CI is based on the preferred term risk difference (Active - Placebo). CIs were only produced if at least one treatment group had at least 4 subjects with the preferred term AE.

In placebo-controlled studies in chronic sinusitis, of those subjects reporting at least 1 TEAE, most reported either mild or moderate TEAEs. Overall, the incidence of subjects reporting severe TEAEs was low in each of the active treatment and placebo groups (2.2% [low dose] and 2.7% [high dose] vs 3.2% [placebo]) with relatively comparable numbers of subjects in each treatment group reporting TEAEs of mild (21.7% [low dose] and 24.6% [high dose] vs 17.6% [placebo]) or moderate (20.1% [low dose] and 24.6% [high dose] vs 26.7% [placebo]) severity.

As with other safety evaluations, the pattern of severity of TEAEs among CS subjects without baseline NP was similar to the pattern observed in the total population. Overall, 52/118 (44.1%), 48/114 (42.1%), and 61/113 (54.0%) subjects without NPs in the placebo, 186 μg OPN-375, and 372 μg OPN-375 groups, respectively, experienced at least 1 TEAE of any severity. Epistaxis, which was the most commonly reported TEAE in OPN-375-treated CS subjects who did not also have NP, was primarily mild in severity (12/17 subjects in all OPN-375 group), and no TEAEs of epistaxis were severe.

In placebo-controlled clinical studies in chronic sinusitis, treatment-related TEAEs occurred in 9.8% of subjects in the OPN-375 group compared with 7.0% of subjects in the placebo group. The highest percentage of subjects with at least 1 treatment-related TEAE was in the 372 μg OPN-375 group (10.9%). The most commonly reported treatment-related TEAE in the combined active treatment group was epistaxis.

The incidence of treatment-emergent SAEs was low overall and similar across the 3 treatment groups (1.6%, 1.1%, and 3.3% in the placebo, 186 μg OPN-375 and 372 μg OPN-375 groups, respectively). None of the SAEs reported in patients with CS were considered related to treatment.

In placebo-controlled phase 3 studies in chronic sinusitis, a total of 4 (2.1%) placebo-treated subjects and 7 (1.9%) OPN-375-treated subjects (4 [2.2%] in the 186 μg OPN-375 and 3 [1.6%] in the 372 μg OPN-375 group) experienced at least 1 TEAE leading to discontinuation of study treatment. No clinically meaningful differences were noted between treatment groups.

Discussion

In both trials, subjects who received either dose of OPN-375 had statistically significantly greater improvements in CSS (Table 51) with symptom improvements continuing through the last CSS assessment at Week 12. In both trials, subjects who received either dose of OPN-375 also had statistically significantly greater improvements from baseline in sinus opacification. The LS mean difference in sinus opacification at Week 24 in the OPN-375 186 mcg group versus exhalation delivery system (EDS)-placebo was −3.98 (95% CI: −7.86, −0.09) in Study A and −8.19 (95% CI: −12.93, −3.45) in Study B. The LS mean difference in sinus opacification at Week 24 in the OPN-375 372 group versus EDS-placebo was −4.59 (95% CI: −8.41, −0.78) in Study A and −6.33 (95% CI: −11.08, −1.58) in Study B. The effects of OPN-375 on both coprimary endpoints were consistent in subjects with or without prior sinus surgery.

In both studies, each dose of OPN-375 significantly improved the loss of sense of smell as early as the first assessment at Week 4, with improvements continuing through the last assessment at Week 12 (Table 6, Table 40). The LS mean difference in loss of sense of smell for the OPN-375 186 mcg twice daily group versus EDS-placebo at Week 4 was −0.35 (95% CI: −0.38, −0.04) in Study A and −0.24 (95% CI: −0.30, 0.07) in Study B and, at Week 12, was −0.60 (95% CI: −0.51, −0.08) in Study A and −0.49 (95% CI: −0.40, 0.08) in Study B. At Week 4, the LS mean difference in loss of smell for the OPN-375 372 mcg twice daily group versus EDS-placebo was −0.35 (95% CI: −0.38, −0.04) in Study A and −0.36 (95% CI: −0.41, −0.05) in Study B and, at Week 12, was −0.54 (95% CI: −0.45, −0.01) in Study A and −0.63 (95% CI: −0.54, −0.07) in Study B.

In both studies, each dose of OPN-375 significantly decreased sino-nasal symptoms and symptom impact as measured by SNOT-22 as early as the first assessment at Week 4, with improvements continuing through Week 24 (Table 48). At Week 24, the LS mean difference in SNOT-22 for the OPN-375 186 mcg twice daily group versus EDS-placebo was −7.89 (95% CI: −12.57, −3.21) in Study A and −10.72 (95% CI: −16.79, −4.65) in Study B. The LS mean difference in SNOT-22 at Week 24 in the OPN-375 372 mcg twice daily group versus EDS-placebo was −12.61 (95% CI: −17.32, −7.90) in Study A and −6.81 (95% CI: −12.92, −0.71) in Study B.

In pre-specified multiplicity-adjusted pooled efficacy analysis of Trials 3 and 4 (n=547), treatment with OPN-375 resulted in significant reductions in acute exacerbations of CRS (Table 30). Through Week 24, the proportion of patients who experienced an acute exacerbation of CRS was reduced by 56% (IRR of 0.441; 95% CI 0.233, 0.832) and 65% (IRR of 0.343; 95% CI 0.173, 0.680) among patients using OPN-375 186 mcg and OPN-375 372 mcg twice daily, respectively, versus EDS-placebo.

In pre-specified multiplicity-adjusted pooled efficacy analysis of patients using a standard-delivery intranasal steroid spray at study entry (n=280), treatment with OPN-375 significantly reduced CSS (Table 32). Through Week 4, the LS mean difference in CSS for the OPN-375 186 mcg twice daily group versus EDS-placebo was −1.53 (95% CI: −1.23, −0.30) and, for the OPN-375 372 mcg twice daily group, was −1.40 (95% CI: −1.10, −0.16).

In addition, treatment with OPN-375 significantly reduced global PSQI scores from baseline through Week 24 (Table 42). At Week 24, the LS mean difference in PSQI for the OPN-375 186 mcg twice daily group versus EDS-placebo was −1.49 (95% CI: −1.30, −0.13). The LS mean difference in PSQI at Week 24 in the OPN-375 372 mcg twice daily group versus EDS-placebo was −1.49 (95% CI: −1.30, −0.13).

Treatment with OPN-375, at doses of either 1 or 2 sprays per nostril (186 or 372 μg) BID, was well tolerated and significantly improved symptoms and sinus opacification in patients with chronic sinusitis. Supportive results from a broad range of additional endpoints, including individual symptoms, disease-specific and general health-related quality of life, acute exacerbations of CRS, and subjective and objective measures of surgical eligibility, all further substantiate the clinical importance of the improvements in disease burden produced by OPN-375 treatment. Improvements in intra-sinus disease, as measured by opacification on CT scan, demonstrate that treatment reduces inflammation inside the sinus cavities, and not simply in the nasal cavity, and this benefit cannot be attributed to treatment of nasal polyps.

Prior to these studies, the broad range of statistically and clinically significant benefits were not known and the benefit/risk ratio for treatment of chronic sinusitis with OPN-375 seems favorable and unexpected in the treatment of patients with chronic sinusitis, in view of the delivery of a steroid, e.g., fluticasone propionate, to the upper posterior region of a subject's nasal cavity achieving a reduction in sinus inflammation, reduction in symptoms, and improvement in the condition of chronic sinusitis. This appears particularly strong in view of the fact that there are no approved drug products for treatment of chronic sinusitis and when considering the alternatives currently considered by physicians.

Surprisingly, patients receiving the method of treatment disclosed herein (i.e., delivery of fluticasone propionate to the upper posterior region of a nasal airway), on average, exhibited an objective reduction in sinus inflammation, irrespective of patient disposition (e.g., gender, age, presence or absence of nasal polyps, prior sinus surgery status, etc.). Similarly, patients receiving the method of treatment disclosed herein (i.e., delivery of fluticasone propionate to the upper posterior region of a nasal airway), exhibited a marked reduction in the incidence of AECRS (approximately 61%), irrespective of patient disposition.

As previously discussed, nasal polyps may be associated with sinus inflammation and CRS/CS. However, it was surprising that the improved therapeutic effects resulting from the method of treatment described herein, namely an objective decrease in sinus inflammation and decreased incidence of AECRS, as well as subjective improvement across an array of symptoms and outcome variables, was achieved irrespective of the presence or absence of nasal polyps. Therefore, this result suggests that the reduction in sinus inflammation and reduced incidence of AECRS achieved do not come from treating nasal polyps, and are therefore efficacious in CRS/CS patients having sinus inflammation as well as CRSwNP patients.

As previously described, sinus surgery is a surgical procedure to remove blockages and treat other conditions, e.g., narrow or narrowing of the sinuses, that a subject may have in the sinuses. Sinus surgery opens the paranasal sinuses, allowing for increased access to the sinuses in an attempt to reduce sinus inflammation. Many patients (estimated over 600,000 per year) undergo surgery that is associated with a variety of risks. While surgery can result in symptom relief, a large proportion of patients continue to require medical therapy after surgery for either incomplete symptom control or recurrent symptoms. While standard nasal administration of a therapeutic agent will not result in topical administration to the sinuses, nasal administration of a therapeutic agent to a patient having undergone sinus surgery will likely result in some topical administration to the sinuses, which may result in an improved therapeutic effect. It was unexpected, however, that the method of treatment described herein would result in an objective reduction of sinus inflammation and reduction in the incidence of AECRS, irrespective of prior sinus surgery status (i.e., patients with or without prior sinus surgery), because sinus surgery would increase access to the sinuses and the chances for topical administration in the sinuses to reduce inflammation, compared to those patients without prior sinus surgery. Because the objective reductions in inflammation and reduction in the incidence of AECRS were achieved in all patients, irrespective of prior sinus surgery status, the method of treatment described herein provides for improved methods of reducing sinus inflammation and the incidence of AECRS in CRS patients, without the need for sinus surgery.

As described above, the present disclosure provides a method of treating a patient. The treatment can include one or more steps, wherein a first step can include administering a therapeutic agent. A second step can include delivering carbon dioxide or a pH adjusting material to one or more regions of the nasal passage, as described above. This effect associated with carbon dioxide when using the breath powered nasal delivery devices may, in combination with one or more other factors, include positive air pressure, high flow rate and changed flow pattern of gasses through a subject's nasal cavity and/or sinuses, improved gas flow, e.g., exhalation breath, penetrating a nasal airway, vibratory effect in operating the devices, and removal of nitric oxide in a subject's nasal cavity and/or sinuses can cause stimulatory or mediating effects on the trigeminal nerve and on mast cells.

In some embodiments, the method of treating described herein is performed for about 1 day, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks.

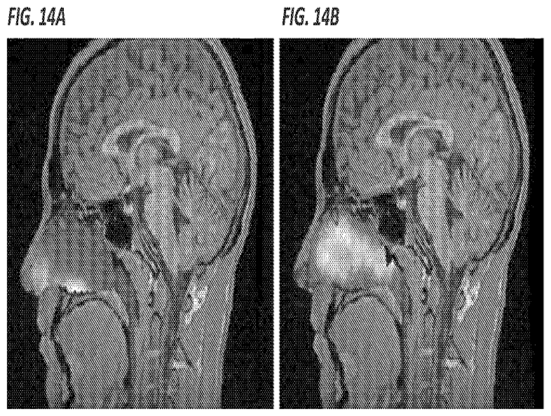

The present disclosure also provides a method of treating chronic rhinosinusitis with or without nasal polyps in a patient in need thereof. The method includes delivering fluticasone propionate to the upper posterior region of a nasal airway in the subject. In some embodiments, the method comprises delivering the fluticasone propionate using a delivery device configured to establish a bi-direction flow of gas flow, e.g., exhalation breath, through a subject's nasal cavity. That is, a device configured to deliver gas flow, e.g., exhalation breath, from a subject into one nasal cavity via a first nostril and further configured to permit the delivered gas flow, e.g., exhalation breath, to flow into the other nasal cavity and exit the subject via a second nostril. In some embodiments, the method comprises delivering fluticasone propionate using an exhalation delivery device as herein described. In some embodiments, the exhalation delivery device comprises a nosepiece that is inserted into a subject's nostril and a mouthpiece that is gripped by the subject's lips. To bi-directionally deliver the dose of fluticasone propionate to the upper posterior region of a nasal airway, there is a simultaneous or near simultaneous (e.g., within milliseconds or seconds) release of a dose of fluticasone propionate in the exhalation delivery device while providing a gas flow, e.g., exhalation breath, through the mouthpiece. It is contemplated that the one or more doses of fluticasone propionate may be delivered in this manner. It is also contemplated that a first dose may be delivered via a first nostril and a subsequent dose may be delivered via a second nostril, i.e., two doses may be delivered to a patient in need thereof one dose per nostril. It is further contemplated that two or more doses may be administered via the same nostril. Administration of a therapeutic agent using the exhalation delivery device as herein described results in increased deposition in the upper posterior region, whereas administration of a therapeutic agent using a standard-delivery nasal spray results in an initial deposition in the lower anterior regions of the nose, and not the upper posterior region of a nasal airway (see FIGS. 14A-14B).

In some embodiments, the fluticasone propionate is administered in about 93 μg per spray or dose. In some embodiments, one spray (about 93 μg) is administered in each nasal cavity for a total dose of about 186 μg. In some embodiments, two sprays (about 186 μg) are administered in each nasal cavity for a total dose of about 372 μg. In some embodiments, the fluticasone propionate (either one spray or two sprays) is administered twice daily (BID) so that a total daily dose of about 372 μg or about 744 μg is administered.

In some embodiments, the described method of administering fluticasone propionate to the upper posterior region of a nasal airway in a subject results in a reduction of inflammation in the subject's nasal cavity. In some embodiments, the described method of administering fluticasone propionate to the upper posterior region of a nasal airway in a subject results in a reduction of inflammation (e.g., a decrease in the average of the percentages of opacified volume in ethmoid and maxillary sinuses) of about 2.86%, about 7.26%, or about 10.53%. In some embodiments, the described method of administering fluticasone propionate to the upper posterior region of a nasal airway in a subject results in clinically meaningful improvement in the reduction of inflammation (e.g., a decrease in the average of the percentages of opacified volume in ethmoid and maxillary sinuses) in approximately 54% of subjects. In some embodiments, the method of treatment described herein results in a decrease of one or more symptoms, including facial pain or pressure, nasal congestion/blockage, rhinorrhea, and sense of smell. In some embodiments, the method of treatment results in a reduction of the incidence of acute exacerbation of CRS (AECRS). In some embodiments, the described method of administering fluticasone propionate to the upper posterior region of a nasal airway in a subject results in a reduction in the incidence of AECRS of about 61% relative to subjects not receiving administration of fluticasone propionate by the exhalation delivery device as disclosed herein. In some embodiments, the method of treatment results in an improvement in scoring in one or more of individual symptom scores, Sinonasal outcome test-22 (SNOT-22), Pittsburgh sleep quality index (PSQI), and patient global impression of change (PGIC). In some embodiments, the method of treatment described herein results in a self-reported improvement of disease in about 80%, or about 90% of patients. In some embodiments, the method of treatment described herein results in subjective (symptoms) and objective (sinus opacification) benefits for patients having CRS.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

All references cited herein are incorporated by reference in their entirety. To the extent publications and patents or patent applications incorporated by reference contradict the disclosure contained herein, the specification will supersede any contradictory material.

What is claimed is:

1. A method for treating chronic rhinosinusitis or chronic sinusitis in a subject in need thereof, comprising bi-directionally delivering, via an exhalation delivery device, a dose of about 186 μg of fluticasone propionate BID to the upper posterior region of a nasal airway in the subject.

2. A method for treating chronic rhinosinusitis or chronic sinusitis in a subject in need thereof, comprising bi-directionally delivering, via an exhalation delivery device, a dose of about 372 μg of fluticasone propionate BID to the upper posterior region of a nasal airway in the subject.

3. The method of claim 1, further comprising inserting a nosepiece into the subject's nasal passage and a mouthpiece that is gripped by the subject's lips.

4. The method of claim 2, further comprising inserting a nosepiece into the subject's nasal passage and a mouthpiece that is gripped by the subject's lips.

5. The method of claim 3, further comprising exhaling through the mouthpiece, the exhalation breath providing for delivery of a dose of the fluticasone propionate.

6. The method of claim 4, further comprising exhaling through the mouthpiece, the exhalation breath providing for delivery of a dose of the fluticasone propionate.

7. The method of claim 1, comprising delivering a dose of about 93 μg of fluticasone propionate in each nasal cavity BID.

8. The method of claim 2, comprising delivering a dose of about 186 μg of fluticasone propionate in each nasal cavity BID.

9. The method of claim 1, wherein delivering the fluticasone propionate to the subject's upper posterior region of a nasal passageway results in a reduction in inflammation and/or a reduction in sinus opacification.

10. The method of claim 2, wherein delivering the fluticasone propionate to the subject's upper posterior region of a nasal passageway results in a reduction in inflammation and/or a reduction in sinus opacification.

11. The method of claim 9, wherein delivering the fluticasone propionate to the subject's upper posterior region of a nasal passageway further results in the improvement of one or more of facial pain or pressure, nasal congestion/blockage, rhinorrhea, and sense of smell.

12. The method of claim 10, wherein delivering the fluticasone propionate to the subject's upper posterior region of a nasal passageway further results in the improvement of one or more of facial pain or pressure, nasal congestion/blockage, rhinorrhea, and sense of smell.

13. The method of claim 1, wherein delivering the fluticasone propionate to the subject's upper posterior region of a nasal passageway results in a reduction of incidence of acute exacerbations of chronic rhinosinusitis (AECRS).

14. The method of claim 2, wherein delivering the fluticasone propionate to the subject's upper posterior region of a nasal passageway results in a reduction of incidence of acute exacerbations of chronic rhinosinusitis (AECRS).

15. The method of claim 1, wherein the fluticasone propionate is administered for about 24 weeks.

16. The method of claim 2, wherein the fluticasone propionate is administered for about 24 weeks.
